(19) United States
(12) Patent Application Publication
(10) Pub. No.: US 2005/0143472 A1
(43) Pub. Date: Jun. 30, 2005

(54) Novel treatment for pathologies associated with oxidative damage

(76) Inventors: Tianyun Wu, San Francisco, CA (US); William S. McIntire, Daly City, CA (US)

Correspondence Address: Christine A Lekutis;Medlen & Carroll, 101 Howard Street, Suite 350, San Francisco, CA, 94105, US

(21) Appl. No.: 10/504,601

(22) PCT Filed: Feb. 13, 2003
(86) PCT No.: PCT/US03/04639

(30) Foreign Application Priority Data
Feb. 13, 2002 (US) 60356851

(51) Int. Cl. C12Q 1/68; C07H 21/04; C12N 9/06; A61K 31/13

(52) U.S. Cl. 514/659; 435/6; 435/69.1; 435/191; 435/320.1; 435/325; 530/388.26; 536/23.2

(57) ABSTRACT

This invention relates to compositions and methods for the treatment of pathologies associated with intracellular polyamine dysregulation. In particular, the present invention provides compositions and methods involving mammalian polyamine oxidase (PAO) to treat cancer, cell damage, tissue damage caused by ischemia/reperfusion, inflammation, traumatic brain injury, stroke and tissue developmental disorders. Methods for diagnosis and prognosis of cancer and other diseases are also provided by the present invention.

This invention was made, in part, with Government support by the Department of Veterans Affairs and the Heart, Lung, and Blood Institute of the National Institutes of Health, Grant No. HL16251. Accordingly, the Government has certain rights in the invention.

### FIELD OF THE INVENTION

This invention relates to compositions and methods for the treatment of pathologies associated with intracellular polyamine dysregulation. In particular, the present invention provides compositions and methods involving mammalian polyamine oxidase (PAO) to treat cancer, cell damage, tissue damage caused by ischemia and reperfusion, inflammation, traumatic brain injury, stroke, and tissue developmental disorders. Methods for diagnosis and prognosis of cancer and other diseases are also provided by the present invention.

### BACKGROUND OF THE INVENTION

Diamine, putrescine (Put) and the polyamines, spermine (Spm) and spermidine (Spd) have been implicated in numerous fundamentally important cellular processes, including wound healing, tissue differentiation, and tumor growth (Seiler, Prog. Brain Res. 106:333-344 [1995]; Wallace, Biochem. Soc. Trans. 26:569-571 [1998]; and Morgan, Biochem. Soc. Trans. 26:586-571 [1998]). A high level of these amines is associated with cell growth, while a decrease in their levels is associated with growth inhibition and cell death. The intracellular polyamine level is strictly regulated by the modulation of enzymes involved in their biosynthesis, catabolism and transport (Pegg, Biochem. J. 234:249-262 [1986]; and Casero and Pegg, FASEB J. 7:652-661 [1993]).

Lowered levels of Put, Spm, and Spd, result from the inhibition of ornithine decarboxylase (ODC) or polyamine oxidase (PAO) (Hölttä, Biochemistry 16:91-100 [1977]; Bolkenius and Seiler, Int. J. Biochem. 13:287-292 [1981]; Hölttä, Methods Enzymol. 94:306-311 [1983]; Seiler, supra [1995]), or from high acetyl Coenzyme A: spermidine/spermine-N¹-acetyltransferase (SSAT) activity. In cells of higher organisms, ODC converts ornithine to Put, which is then converted to Spd by Spd synthase. Spd in turn is converted to Spm by Spm synthase. SSAT acetylates Spd and Spm for excretion from cells or for oxidation by the peroxisomal flavoprotein PAO (van den Munckhof et al., J. Histochem. Cytochem. 43:1155-1162 [1995]). PAO oxidizes N¹-acetyl-Spm to 3-acetamidopropanal and Spd, and N¹-acetyl-Spd to 3-acetamidopropanal and Put. Spd and Put return to the intracellular polyamine pool, and 3-acetamidopropanal can be deacetylated to form the cytotoxin, 3-aminopropanal (Houen et al., Acta Chem. Scand. 48:52-60 [1994]). Importantly, 3-aminopropanal is thought to contribute, either alone or in concert with H₂O₂, to tissue damage following traumatic or ischemic injury (Ivanova et al., J. Exp. Med. 188:327-340 [1998]; Dogan et al., J. Neurosurg. 90:1078-1082 [1999]; and Dogan et al., J. Neurochem. 72:765-770 [1999]; Ivanova et al., Proc. Natl. Acad. Sci. USA 99:5579-5584 [2002]). On exposure to substrate, the flavin adenine dinucleotide (FAD) of PAO becomes reduced. As shown in FIG. 1, the reduced FAD is reoxidized by O₂ to regenerate active PAO and to produce hydrogen peroxide. Through production of hydrogen peroxide, PAO regeneration has been proposed to play a role in triggering and/or participating in the progression of apoptosis (Hu and Pegg, Biochem J. 328:307-316 [1997]; Kramer et al., Cancer Res. 59:1278-1286 [1999]; Mank-Seymour et al., Clin. Cancer Res. 4:2003-2008 [1998]; Lindsay and Wallace, Biochem. J. 337:83-87 [1999]; and Chopra and Wallace, Biochem Pharmacol. 55:1119-1123 [1998]).

Thus, mammalian PAO has clinical and pharmacological relevance to various pathological conditions. Clearly, there is a need in the art for the development of molecular and biochemical tools to produce PAO-based compositions and methods for diagnostic, prognostic and therapeutic applications.

### SUMMARY OF THE INVENTION

This invention relates to compositions and methods for the treatment of pathologies associated with intracellular polyamine dysregulation. In particular, the present invention provides compositions and methods involving mammalian polyamine oxidase (PAO) to treat cancer, cell damage, tissue damage caused by ischemia and reperfusion, inflammation, traumatic brain injury, stroke, and tissue developmental disorders. Methods for diagnosis and prognosis of cancer and other diseases are also provided by the present invention.

The present invention provides isolated nucleic acids that comprise an open reading frame for a peroxisomal polyamine oxidase of a mammal. In preferred embodiments, the mammal is selected from the group consisting of cattle, mice and humans. In particularly preferred embodiments, the isolated nucleic acid is selected from the group consisting of SEQ ID NO:1, SEQ ID NO:3, and SEQ ID NO:5. In some embodiments the isolated nucleic acid comprises deoxyribonucleic acid.

In addition, the present invention provides vectors comprising at least one isolated nucleic acid selected from the group consisting of SEQ ID NO:1, SEQ ID NO:3, and SEQ ID NO:5. In some embodiments, the vector further comprises a promoter and an operator operatively linked to the nucleic acid. In some preferred embodiments the vector is a bacterial expression vector.

The present invention also provides compositions comprising at least one recombinant peroxisomal polyamine oxidase of a mammal. In preferred embodiments, the mammal is selected from the group consisting of cattle, mice and humans. In some particularly preferred embodiments, the recombinant peroxisomal polyamine oxidase comprises a sequence selected from the group consisting of SEQ ID NO:4 and SEQ ID NO:6. In other embodiments, the recombinant peroxisomal polyamine oxidase further comprises a polyhistidine tag.

In some embodiments, the present invention provides host cells transformed with a vector comprising a nucleic acid selected from the group consisting of SEQ ID NO:1, SEQ ID NO:3, and SEQ ID NO:5. In some preferred embodiments, the host cell is a bacterial cell. In some particularly preferred embodiments, the bacterial cell is an E. coli cell.

The present invention also provides compositions comprising the polyamine oxidase produced by a host cell transformed with a vector comprising a nucleic acid selected from the group consisting of SEQ ID NO:1, SEQ ID NO:3, and SEQ ID NO:5. In some embodiments, the composition further comprises ethylene glycol. In some preferred embodiments, the polyamine oxidase of the composition further possesses enzymatic activity.

In addition, the present invention provides methods for detecting polyamine oxidase expression in a cell comprising the steps of: a) providing: at least one cDNA generated from mRNA harvested from the cell, at least one polyamine oxidase primer pair, and at least one control primer pair; b) annealing the cDNA with the polyamine oxidase primer pair and amplifying the cDNA under conditions such that an amplified polyamine oxidase DNA fragment is obtained; and c) annealing the cDNA with the control primer pair such that an amplified control DNA fragment is obtained. In some embodiments, the amplification is conducted by polymerase chain reaction. In some embodiments, the method further comprises electrophoresis of the amplified DNA fragments through an agarose gel. In related embodiments, the method further comprises staining the amplified DNA fragments within the gel with ethidium bromide and measuring the fluorescence intensity of the ethidium bromide-stained DNA fragments. In some preferred embodiments, the methods further comprise step d) comparing the fluorescence intensity of the ethidium bromide-stained polyamine oxide DNA fragment to the ethidium bromide-stained control DNA fragment.

Moreover, the present invention provides isolated nucleic acids that comprises a sequence selected from the group consisting of a gene encoding a peroxisomal acetylpolyamine oxidase protein of a mammal, a gene encoding a biologically active portion of the peroxisomal acetylpolyamine oxidase protein, and a gene encoding a biologically active variant of the peroxisomal acetylpolyamine oxidase protein. In some embodiments, the mammal is selected from the group consisting of a cow, a mouse and a human. In related embodiments, the nucleic acid is selected from the group consisting of the open reading frames of SEQ ID NO:1, SEQ ID NO:3, and SEQ ID NO:5. In further embodiments, the nucleic acid encodes a protein selected from the group consisting SEQ ID NO:2, SEQ ID NO:4, and SEQ ED NO:6. In some preferred embodiments, the nucleic acid encodes a protein with amine oxidizing activity. In a subset of these embodiments, the substrate for the amine oxidizing activity is selected from the group consisting of N¹-acetyl-Spm, N¹-acetyl-Spd, N¹,N¹²-diethyl-Spm, N¹,N¹¹-diethyl-nor-Spm, and Spm. Also provided are vectors comprising an isolated nucleic acid sequence selected from the group consisting of a gene encoding a peroxisomal acetylpolyamine oxidase protein of a mammal, a gene encoding a biologically active portion thereof, and a gene encoding a biologically active variant thereof. In some embodiments, the vector further comprises a promoter operatively linked to the nucleic acid. Additionally, the present invention provides host cells transformed with a vector comprising an isolated nucleic acid sequence selected from the group consisting of a gene encoding a peroxisomal acetylpolyamine oxidase protein of a mammal, a gene encoding a biologically active portion thereof, and a gene encoding a biologically active variant thereof. In some embodiments, the host cell is located in an animal. In other embodiments, the present invention provides a host cell comprising a disruption of a gene encoding a peroxisomal acetylpolyamine oxidase protein of a mammal, a gene encoding a biologically active portion thereof, and a gene encoding a biologically active variant thereof. In some embodiments, the host cell is located in an animal.

In some embodiments, the present invention provides isolated mammalian nucleic acid sequences selected from the group consisting of the open reading frames of SEQ ID NO:1, SEQ ID NO:3, SEQ ID NO:5 and sequences that hybridize to the complement of the open reading frames of SEQ ID NO:1, SEQ ID NO:3, SEQ ID NO:5, under conditions of low stringency, and wherein the isolated nucleic acid sequence encodes a polypeptide having amine oxidizing activity.

In other embodiments, the present invention provides compositions comprising an isolated protein selected from the group consisting of a peroxisomal acetylpolyamine oxidase of a mammal, a biologically active portion thereof, or a biologically active variant thereof. In some preferred embodiments, the mammal is selected from the group consisting of a cow, a mouse and a human. In a subset of these embodiments, the peroxisomal acetylpolyamine oxidase comprises a sequence selected from the group consisting of SEQ ID NO:2, SEQ ID NO:4, and SEQ ID NO:6. In some preferred embodiments, the peroxisomal acetylpolyamine oxidase has amine oxidizing activity. In some of these embodiments, the substrate for the amine oxidizing activity is selected from the group consisting of N¹-acetyl-Spm, N¹-acetyl-Spd, N¹,N¹²-diethyl-Spm, N¹, N¹¹-diethyl-nor-Spm, and Spm. Moreover, the present invention provides embodiments wherein the peroxisomal acetylpolyamine oxidase is a recombinant peroxisomal acetylpolyamine oxidase protein. In related embodiments, the peroxisomal acetylpolyamine oxidase protein further comprises an affinity tag.

The present invention provides methods for detecting mammalian peroxisomal acetylpolyamine oxidase expression in a cell comprising the steps of: providing: i) a sample from a mammalian subject, and ii) at least one reagent capable of specifically detecting mammalian peroxisomal acetylpolyamine oxidase expression; and contacting the sample with at least one reagent under conditions suitable for binding at least one reagent to a mammalian peroxisomal acetylpolyamine oxidase gene product. In embodiments in which the mammalian peroxisomal acetylpolyamine oxidase gene product comprises mRNA, at least one reagent comprises a nucleic acid probe of at least 12 nucleotides in length that specifically hybridizes under conditions of high stringency to the mRNA or to cDNA corresponding to the mRNA. In related embodiments, the contacting is accomplished by a technique selected from the group consisting of polymerase chain reaction and Northern blotting. In embodiments in which the mammalian peroxisomal acetylpolyamine oxidase gene product comprises protein, the at least one reagent comprises an antibody that binds to the protein. In related embodiments, the contacting is accomplished by a technique selected from the group consisting of enzyme-linked immunosorbent assay, Western blotting, immunofluorescence analysis, immunohistochemistry and flow cytometry. In a subset of these embodiments, the antibody further comprises a reporter molecule selected from the group consisting of an enzyme and a fluorochrome.

Moreover the present invention provides methods of inhibiting mammalian peroxisomal acetylpolyamine oxidase activity comprising: providing a mammalian peroxisomal acetylpolyamine oxidase, and an inhibitor; and contacting the mammalian peroxisomal acetylpolyamine oxidase with the inhibitor under conditions suitable for reducing amine oxidizing activity of the oxidase. In some preferred embodiments, the inhibitor is selected from the group consisting of synthalin and N-(3-aminopropyl)-1,10 decanediamine. In further embodiments, the mammalian peroxisomal acetulpolyamine oxidase is located in a cell or in an animal.

Additionally, the present invention provides methods comprising: providing a host cell comprising an exogenous nucleic sequence selected from the group consisting of the open reading frames of SEQ ID NO:1, SEQ ID NO:3, SEQ ID NO:5 and sequences that hybridize to the complement of the open reading frames of SEQ ID NO:1, SEQ ID NO:3, SEQ ID NO:5 under conditions of low stringency, wherein the isolated nucleic acid sequence encodes a polypeptide having amine oxidizing activity; and culturing the host cell under conditions such that the exogenous nucleic acid sequence is expressed.

### DESCRIPTION OF THE FIGURES

The following Figures form part of the Specification and are included to further demonstrate certain aspects and embodiments of the present invention. The invention may be better understood by reference to one or more of these Figures in combination with the detailed description of specific embodiments presented herein.

FIG. 1 depicts substrate oxidation by PAO. Panel A shows N¹-acetyl-Spm oxidation by PAO, while panel B shows N¹-acetyl-Spd oxidation by PAO. FADH₂ is the two-electron reduced form of the enzyme bound FAD, while R stands for the ribityl-diphosphoadenine moiety of FAD. Also shown is the in vivo deacetylation of 3-acetamidopropanal to the cytotoxin, 3-aminopropanal, and the conversion of H₂O₂ to H₂O and O₂ by catalase.

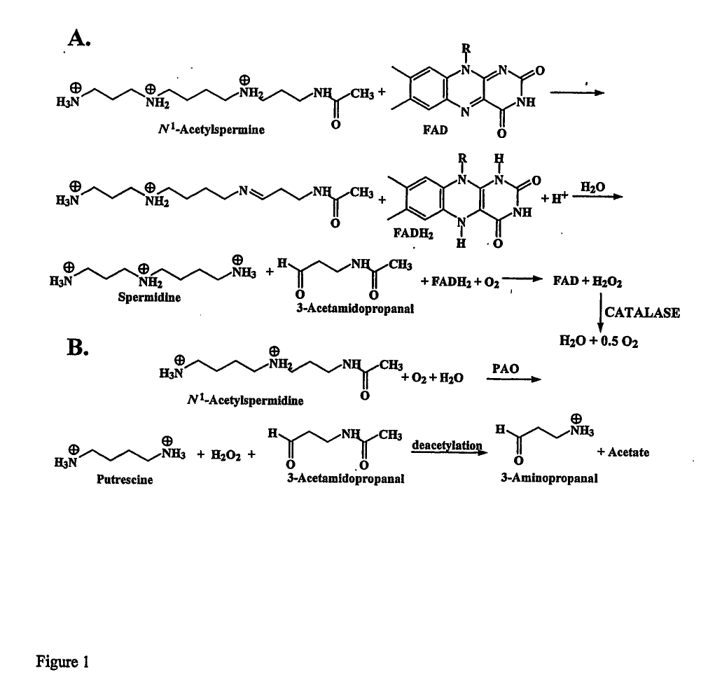

FIG. 2 shows the partial bovine PAO (bPAO) cDNA (SEQ ID NO:1) and amino acid sequences (SEQ ID NO:2). The dashes indicate a segment of the protein sequence of unknown length and composition, the asterisk denotes the translation stop codon, and the double underline denotes the peroxisomal transport signal. The three single-underlined amino acid segments were derived by protein sequencing of the intact enzyme (SEQ ID NO:7) and two tryptic peptides (SEQ ID NO:8; and SEQ ID NO:9).

FIG. 3 shows the murine PAO (mPAO) cDNA (SEQ ID NO:3) and amino acid sequences (SEQ ID NO:4). The + symbol marks the start of exon I. This base (No. 18) corresponds to base No. 7477 of SEQ ID NO:21 and base No. 1069500 of GenBank Accession No. NW₁₃ 000335 (gene transcript ID No. XM 133921.1). The double-underline at the 5′-end represents sequence from the cloning vector, the asterisk denotes the translation stop codon, and the single-underline denotes the peroxisomal transport signal.

FIG. 4 shows the human (hPAO) PAO cDNA (SEQ ID NO:5) and amino acid sequences (SEQ ID NO:6). The + symbol represents the start of the exon I. This base (No. 27) corresponds to base No. 2840 of SEQ ID NO:14 and base No. 83941 of GenBank Accession No. AL360181.31. The double-underline at the 5′-end represents sequence from the cloning vector, the asterisk denotes the translation stop codon, and the single-underline denotes the peroxisomal transport signal.

FIG. 5 shows an alignment of the human (complete), murine (complete) and bovine (partial) PAO amino acid sequences. The underlined segments of the bovine sequence indicate the sequences derived from Edman degradation analysis of the intact protein (amino-terminus) (SEQ ID NO:8) and two pure peptides (SEQ ID NO:9 and SEQ ID NO:10). The double-underlined bPAO segment indicates the overlap of information from peptide sequencing and from translation of the cDNA sequence. At the C-termini, the Pro-Arg-Leu motif is a peroxisomal transport signal. The question marks for the bovine sequence indicate a segment of unknown composition.

FIG. 6 provides schematics of human and murine PAO mRNA and genomic DNA. Panel A depicts a schematic of human PAO mRNA. Panel B depicts a schematic of the human PAO genomic DNA, which is located on chromosome 10 and contains 7 exons totaling 1822 bp, including an open reading-frame of 1533 bp. Introns are represented by lines, while exons are represented by boxes with the length of each exon in base pairs listed above. Panel C provides a schematic of the human PAO exons with numbers indicating the locations of the putative splice sites. The number at the beginning of exon I corresponds to base No. 27 of the hpao cDNA sequence in FIG. 4, and base No. 2840 of the hpao genomic sequence of SEQ ID NO:14. The number at the end of exon VII corresponds to base No. 1849 of the cDNA sequence in FIG. 4 and base No. 15266 of the genomic sequence of SEQ ID NO:14. Panel D depicts the location of a deletion in an EST derived from a genitourinary high-grade transitional cell tumor (GenBank Accession No. AW662266), the wild type and mutant human sequences are also disclosed herein as SEQ ID NO:22 and SEQ ID NO:23, respectively. Panel E depicts a schematic of murine PAO mRNA. Panel F shows a schematic of murine PAO genomic DNA (length of 8651 bp), which is located on chromosome 7 and contains 7 exons totaling 1755 bp, including an open reading-frame of 1512 bp. Introns are represented by lines, while exons are represented by boxes with the length of each exon in base pairs listed above. Panel G provides a schematic of murine PAO exons with numbers indicating the locations of putative splice sites. The number at the beginning of exon I correspond to base No. 18 and of the mpao cDNA sequence in FIG. 3, and base No. 7477 of the mpao genomic sequence of SEQ ID NO:21. The number at the end of exon VII corresponds to base No. 1758 of the cDNA sequence in FIG. 3 and base No. 16129 of the genomic sequence of SEQ ID NO:21.

FIG. 7, panel A displays the chromosomal location of the human polyamine oxidase gene as well as a cytogenetic map. FIG. 7, panel B shows the chromosomal location of murine polyamine oxidase gene and a cytogenetic map.

FIG. 8 shows the UV-visible spectrum, recorded at pH 7.6, of recombinant mPAO produced in bacteria.

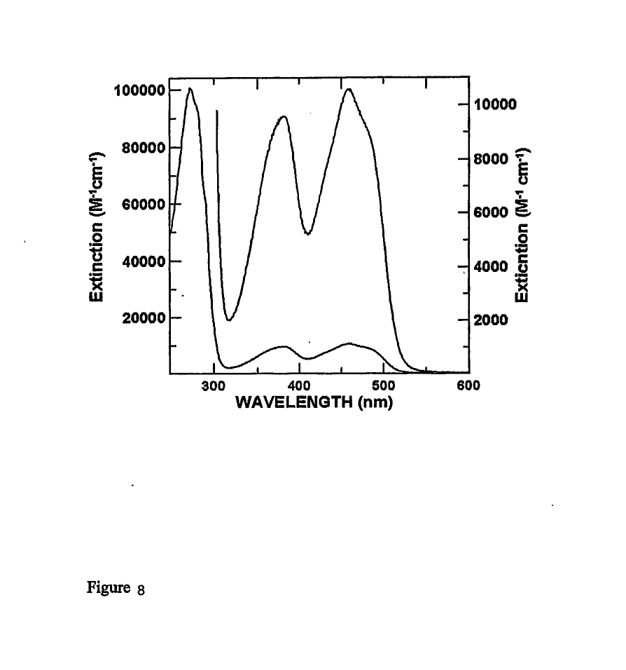

FIG. 9 show the results of a reductive dithionite titration (DT) of mPAO. Panel A shows the spectrum of oxidized mPAO (------), including those obtained at the beginning of the titration after addition of 2.16 and 4.433 nmol DT (solid lines), and that of fully reduced mPAO after addition of 17.3 nmol DT ( - - - ). The arrows indicate the change in absorbance that occurred upon DT addition. Panel B displays the spectral changes that occurred in the latter portion of the titration. The arrows indicate the changes that took place as progressively more DT was added: 4.33, 6.49, 8.66, 10.8, 13.0, 15.1 and 17.3 nmol DT. The inset shows a plot of absorbance versus DT concentration. Panel C displays the spectra of the fully oxidized (dashed line, - - - ) , the radical (straight line, [symbol] ), and the fully reduced (dotted line, ------ ) forms of FAD bound to mPAO, which resulted from the Factor Analysis of the titration data presented in Panels A and B.

FIG. 10 displays structures of substrates and inhibitors of PAO. The asterisks indicate those compounds that are substrates for mPAO.

FIG. 11 shows an agarose gel containing the mPAO (lower frame) and β-actin (upper frame) PCR products amplified from murine cDNA from various tissues and developmental stages. The M denotes the lane containing the DNA standards, and the numbered lanes represent mRNA from the following tissues: 1) brain; 2) heart; 3) kidney; 4) spleen; 5) thymus; 6) liver; 7) stomach; 8) small intestine; 9) muscle; 10) lung; 11) testis; 12) skin; 13) adrenal gland; 14) ovary; 15) uterus; 16) prostate gland; 17) 8.5 day old embryo; 18) 9.5 day old embryo; 19) 12.5 day old embryo; 20) 19 day old embryo; 21) virgin breast; 22) pregnant breast; 23) lactating breast; and 24) involuting breast. Embryo ages are given in days post-conception.

FIG. 12 shows a multiple tissue Northern blot screened with HPAO (upper panel) and β-actin (lower panel) hybridization probes.

FIG. 13 provides a comparison of hpao mRNA expression levels in normal human liver and placenta, and OVCAR-3 and HL-60 cancer cell lines (top panel). GAPDH expression was examined in these same mRNA preparations as a positive control (bottom control).

FIG. 14 shows the toxic effect of N¹-acetyl-Spm on OVCAR-3 and HL-60 cells. The y-axis represents the percentage of surviving cells, while the x-axis represents the final concentration of N¹-acetyl-Spm added to the culture medium.

FIG. 15 shows the predicted secondary structure of mPAO obtained using the “Predict Protein” (PED-sec) and “Psi-Pred” version 2 (Psi-Pred) programs. Also shown is a comparison of these structures with the “refined” secondary structure of the (PDB) three-dimensional model of mPAO determined as described in Example 5. H indicates alpha helix propensity, while E indicates beta-sheet propensity.

FIG. 16 shows a stereoview of the theoretical ribbon structures for a mPAO/MDL 72527 complex (top panel). The arrows point to the Rossmann-fold motif that interacts primarily with the ribityl-ADP portion of FAD. The MDL 72527 inhibitor (bottom panel) is given a positive charge on N10 because there is an apparent strong electrostatic interaction between this ammonium ion and the carboxylate of Glu⁸⁴. The C4-carbon corresponds to the center oxidized in the normal substrate reaction. One of the C4 hydrogens of the inhibitor is closest to the N5-position of FAD (2.59 Å). MDL 72527 is shown in white, FAD is shown in black, the α-helix (residues 475 to 494) that interacts with the N1/C2O locus is shown in dark gray, and the α-helix (residues 14 to 26) that interacts with the diphosphoryl portion of FAD is shown in white.

FIG. 17 shows the stereoviews of theoretical ribbon structures for a mPAO/N¹-acetyl-Spm complex. The top frame shows the modeled structure from the same perspective as that of FIG. 16, while the middle frame shows the same structure viewed down the substrate binding pocket, along the axis of the stretched out substrate. The top part of the structure in the top frame is the flavin-binding domain, while the bottom part is the substrate-binding domain. The substrate N16-nitrogen is directly inside the opening to the binding pocket, while a hydrogen on C6 of the substrate is closest to the flavin N5-position (2.70 Å). There are positive charges on N12 and N16 of the N¹-acetyl-Spm substrate shown in the bottom frame, because there appears to be specific interaction between these amino groups and Glu⁸⁴ and Asp³³⁹. In the stereoviews, the substrate is shown in white, and FAD is shown in black.

### DEFINITIONS

To facilitate understanding of the invention, a number of terms are defined and discussed below.

The term “peroxisome” refers to a small organelle found in the cytoplasm of the cell which houses reactions in which toxic peroxides are formed as unavoidable side products of chemical reactions.

The term “gene” refers to a nucleic acid (e.g., DNA) sequence that comprises coding sequences necessary for the production of a polypeptide or precursor or RNA (e.g., tRNA, siRNA, rRNA, etc.). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the sequences located adjacent to the coding region on both the 5′ and 3′ ends, such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region, which may be interrupted with non-coding sequences termed “introns” or “intervening regions” or “intervening sequences.” Introns are removed or “spliced out” from the nuclear or primary transcript, and are therefore absent in the messenger RNA (mRNA) transcript. The mRNA functions during translation to specify the sequence or order of amino acids in a nascent polypeptide.

In particular, the terms “PAO gene” “polyamine oxidase gene” “peroxisomal polyamine oxidase gene” “acetylpolyamine oxidase gene” “N¹-acetylpolyamine oxidase gene” “N¹-acetyl-spermine/spermidine oxidase gene” and “APAO gene” as used herein, refer to full-length mammalian peroxisomal acetylpolyamine oxidase genes. In some preferred embodiments, the term “PAO gene” refers to the nucleotide sequences disclosed herein as SEQ ID NO:1 (bPAO), SEQ ID NO:3 (mPAO) also disclosed as GenBank Accession No. NM_{—}153783, SEQ ID NO:5 (hPAO), and SEQ ID NO:14 (hPAO). However, it is also intended that the term encompasses fragments of the PAO nucleotide sequence, as well as other domains (e.g., functional domains) within the full-length PAO nucleotide sequence. Furthermore, the terms “PAO gene,” “PAO nucleotide sequence,” and “PAO polynucleotide sequence” encompass DNA, cDNA, and RNA sequences. However, in the context of the present invention, the terms “PAO gene” and “polyamine oxidase gene” do not refer to the mammalian cytosolic polyamine oxidase genes (e.g. GenBank Accession Nos.: NM_{—}019025, AL121675, AY033889,AY033890, AY033891, AF519179, AK000753 and BC004831 disclosed by Wang et al., Cancer Research, 61:5370-5373 [2001]; Murray-Stewart et al., Biochem. J., 368:673-677 [2002]; Vujcic, et al., Biochem. J. 367:665-675 [2002]; and in International Patent Application No. WO 02/100884, herein incorporated by reference). This cytosolic enzyme will be referred to herein as spermine oxidase since it oxidizes Spm but not N¹-acetyl-Spm or N¹-acetyl-Spd, in contrast to APAO.

As used herein, the terms “PAO protein” “polyamine oxidase protein” “peroxisomal polyamine oxidase protein” “acetylpolyamine oxidase protein” “N¹-acetylpolyamine oxidase protein” “N¹-acetyl-spermine/spermidine oxidase protein” and “APAO protein” refer to mammalian peroxisomal acetylpolyamine oxidase proteins, including wild type and mutant PAO proteins, but does not refer to the mammalian Spm oxidase just mention (NCBI Accession Nos. BAA91360, BAA91360, AAK55763 and AAH04831 disclosed by Vujcic, et al. [2002]). The partial bovine PAO protein sequence is set forth as SEQ ID NO:2, the murine PAO protein sequence is set forth as SEQ ID NO:4, and the human PAO protein sequence is set forth as SEQ ID NO:6. Some embodiments of the present invention comprise mammalian homologs of the human PAO protein, which differ from the human PAO protein in fewer than 25% of the residues (e.g., percent sequence similarity). Other embodiments comprise variants of the mammalian PAO proteins, which differ from the wild type PAO sequences in fewer than 1% of the residues (e.g., percent sequence similarity).

As used herein, the terms “open reading frame,” “ORF,” “coding sequence,” and “coding region” refer to the nucleotide sequences that encode the amino acid sequences found in the nascent polypeptide as a result of translation of an mRNA molecule. The coding region is bounded in eukaryotes, on the 5′ side by the nucleotide triplet “ATG” that encodes the initiator methionine and on the 3′ side by one of the three triplets which specify stop codons (i.e., TAA, TAG, and TGA).

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “purified” refers to molecules (polynucleotides or polypeptides) that are removed from their natural environment, isolated or separated. “Substantially purified” molecules are at least 50% free, preferably at least 75% free, and more preferably at least 90% free from other components with which they are naturally associated.

In particular, the term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

Where amino acid sequence is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule. The term “wild-type” refers to a gene or gene product that has the characteristics of that gene or gene product when isolated from a naturally occurring source. A wild type gene is that which is most frequently observed in a population and is thus arbitrarily designed the “normal” or “wild-type” form of the gene.

In contrast, the terms “modified,” “mutant,” and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “complementary” and “complementarity” refer to polynucleotides related by base-pairing rules. For example, for the sequence “5′-AGT-3′,” the complementary sequence is “3′-TCA-5′.”

As used herein the term “portion” when in reference to a nucleotide sequence (as in “a portion of a given nucleotide sequence”) refers to fragments of that sequence. The fragments may range in size from 12 nucleotides to the entire nucleotide sequence minus one nucleotide. In some embodiments, the term portion refers to nucleic acid fragments of at least 24 nucleotides in length. In preferred embodiments, the fragments are at least 48 nucleotides in length, in particularly preferred embodiments, the fragments are at least 96 nucleotides in length.

The term “portion” as used herein when in reference to a protein (as in “a portion of a given protein”) refers to fragments of that protein. The fragments may range in size from four consecutive amino acid residues to the entire amino acid sequence minus one amino acid. In some embodiments, the term portion refers to polypeptides of at least 8 amino acids in length. In preferred embodiments, the polypeptides are at least 16 amino acids in length, in particularly preferred embodiments, the polypeptides are at least 32 nucleotides in length.

As used herein, the term “biologically active” refers to a molecule having structural, regulatory and or biochemical functions of a wild type PAO molecule (e.g., peroxisomal acetylpolyamine oxidase gene or protein). In some instances, the biologically active molecule is a mammalian PAO molecule (e.g., HPAO or its homologs), while in other instance the biologically active molecule is a portion of a mammalian PAO molecule. Other biologically active molecules which find use in the compositions and methods of the present invention include but are not limited to mutant (e.g., variants with at least one deletion, insertion or substitution) mammalian PAO molecules. Biological activity is determined for example, by restoration or introduction of PAO activity in cells which lack PAO activity, through transfection of the cells with a PAO expression vector containing a PAO gene, derivative thereof, or portion thereof. In preferred embodiments, biologically activity is determined by measuring amine-oxidizing activity of the PAO variant of interest using the methods disclosed in Example 5.

The term “conservative substitution” as used herein refers to a change that takes place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartame, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur-containing (cysteine and methionine) (e.g., Stryer ed., Biochemistry, 4th ed., WH Freeman and Co., New York, pp.18-23 [1981]). Whether a change in the amino acid sequence of a peptide results in a functional homolog can be readily determined by assessing the ability of the variant peptide to function in a fashion similar to the wild-type protein. Peptides having more than one replacement can readily be tested in the same manner. In contrast, the term “nonconservative substitution” refers to a change in which an amino acid from one family is replaced with an amino acid from another family (e.g., replacement of a glycine with a tryptophan). Guidance in determining which amino acid residues can be substituted, inserted, or deleted without abolishing biological activity can be found using computer programs (e.g., LASERGENE software, DNASTAR Inc., Madison, Wis.).

The term “mammals” refers to animals of the class mammalia which nourish their young by fluid secreted from mammary glands of the mother, including human beings. The class “mammalian” includes placental animals, marsupial animals, and monotrematal animals. Preferred embodiments of the present invention include a mammalian PAO gene or gene product (e.g., cows, mice, humans, rats, pigs, monkeys, etc.).

As used herein the term “enzyme” refers to a protein which catalyses chemical reactions of other substances without itself being destroyed or altered upon completion of the reactions. Enzymes are divided into six main groups: oxidoreductases, transferases, hydrolases, lyases, isomerases and ligases. The term “enzymatic activity” refers to the catalytic activity of an enzyme or the activity by which the rate of a biochemical reaction is increased without altering the nature or the direction of the reaction. In preferred embodiments of the present invention the term “enzyme” is used in reference to mammalian PAOs.

The terms “polyamine oxidase” and “PAO” refer to an enzyme which catalyzes the oxidative cleavage of the N¹-acetylated polyamines substrates such as N¹-acetyl-spermidine (N¹-acetyl-Spd), N¹-acetyl-spermine (N¹-acetyl-Spm), or other polyamines to produce a shortened polyamine or diamine, hydrogen peroxide and 3-acetamidopropanal. The term “substrate” refers to a substance upon which an enzyme acts.

As used herein, the terms “mammalian peroxisomal acetylpolyamine oxidase inhibitor” “PAO inhibitor” “APAO inhibitor” and “oxidase inhibitor” refer to any compound which can be used to reduce activity of a mammalian peroxisomal acetylpolyamine oxidase. Compounds which reduce APAO activity can be identified using the methods disclosed herein in Example 5. In some embodiments, the term oxidase inhibitor refers to but is not limited to synthalin and N-(3-aminopropyl)-1,10 decanediamine. Some preferred oxidase inhibitors are contemplated to selectively reduce the amine oxidizing activity of peroxisomal acetylpolyamine oxidases (as opposed to also inhibiting the activity of cytosolic polyamine oxidases).

The term “reducing” as used herein in reference to inhibition of mammalian peroxisomal acetylpolyamine oxidase activity refers to conditions suitable to effect a decrease in oxidase activity of at least two-fold as defined by k_{cat} or the “apparent” k_{cat}, which is expressed as μmoles (micromoles) of substrate oxidized per min per mg of enzyme ( i.e., μmol min⁻¹ mg⁻¹, where 1 Unit of activity=1 μmol min⁻¹ mg⁻¹). The values of k_{cat} or the “apparent” k_{cat} are also typically expressed as units of sec⁻¹. The term “apparent” indicates that the kinetic parameter, k_{cat}, was determined at a concentration of the substrate (e.g., O₂) that is subsaturating (e.g., at a concentration that is not at least several times the value of the Michaelis constant for the substrate). In preferred embodiments, the reduction is at least five-fold, more preferably at least ten-fold, and most preferably at least 100-fold.

As used herein, the term “vector” refers to any nucleic acid molecule that can incorporate foreign DNA and transfer it from one cell to another. Vectors are often derived from plasmids, bacteriophages, or plant or animal viruses. Similarly, the term “expression vector” refers to a recombinant DNA molecule containing a desired coding sequence and appropriate nucleic acid sequences necessary for the expression (e.g., transcription and translation) of the operably linked coding sequence in a particular host organism.

The term “plasmid” as used herein, refers to a small, independently replicating, piece of DNA. Similarly, the term “naked plasmid” refers to plasmid DNA devoid of extraneous material typically used to affect transfection. As used herein, a “naked plasmid” refers to a plasmid substantially free of calcium-phosphate, DEAE-dextran, liposomes, and/or polyamines.

As used herein, the term “expression” refers to the process by which a gene's coded information is converted into an operable structure such as an mRNA and/or a protein molecule. Thus, expressed genes are those that are transcribed into mRNA and then translated into protein, as well as those that are transcribed into RNA but not translated into protein (e.g., transfer and ribosomal RNAs).

The term “promoter” as used herein, refers to a DNA nucleotide sequence that when attached to an RNA polymerase molecule, will initiate transcription. Bacterial promoters utilized in some embodiments of the present invention include the T7 and trc promoters. The trc promoter is a hybrid promoter derived from the trp and lac promoters. The term “operator” refers to the site of repressor binding on a DNA molecule. In some embodiments of the present invention the lac operator is employed.

As used herein, the term host cell refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The term “transgene” as used herein, refers to a foreign gene that is placed into an organism by introducing the foreign gene into newly fertilized eggs or early embryos. The terms “foreign gene” and “exogenous gene” refers to any nucleic acid (e.g., gene sequence) that is introduced into the genome of an animal by experimental manipulations and may include gene sequences found in that animal so long as the introduced gene does not reside in the same location as does the naturally-occurring, “endogenous” gene.

The term “transformed host cell” refers to the genetic modification of a cell by incorporation of free DNA. In preferred embodiments, the transformed host cell is a bacterial cell. “Transformation” of bacteria is typically brought about by heat or osmotic shock, electroporation or conjugation with another bacterial species.

The terms “bacteria” and “bacterial” as used herein, refer to prokaryotic organisms (e.g., Archebacteria, Eubacteria, Cyanobacteria). In preferred embodiments, the term “bacteria” refers to Eubacteria, which can be further subdivided on the basis of their staining using Gram stain (e.g., gram-positive and gram-negative).

As used herein, the term “prokaryote” refers to organisms distinguishable from “eukaryotes.” It is intended that the term prokaryote encompass organisms that exhibit the characteristics indicative of prokaryotes, such as possessing a single, circular chromosome, the lack of a true nucleus, the lack of membrane-bound organelles, and other molecular characteristics indicative of prokaryotes.

As used herein, the term “eukaryote” refers to organisms distinguishable from “prokaryotes.” It is intended that the term eukaryote encompass all organisms with cells that exhibit the usual characteristics of eukaryotes such as the presence of a true nucleus bounded by a nuclear membrane within which reside the chromosomes, the presence of membrane-bound organelles, and other characteristics commonly observed in eukaryotic organisms.

The term “recombinant DNA” refers to a DNA molecule that is comprised of segments of DNA joined together by means of molecular biology techniques. Similarly, the term “recombinant protein” refers to a protein molecule that is expressed from recombinant DNA.

The term “fusion protein” as used herein refers to a protein formed by expression of a hybrid gene made by combining two gene sequences. Typically this is accomplished by cloning a cDNA into an expression vector in frame with an existing gene. The fusion partner may act as a reporter (e.g., βgal) or may provide a tool for isolation purposes (e.g., polyhistidine).

Suitable systems for production of recombinant proteins include but are not limited to prokaryotic (e.g., Escherichia coli), yeast (e.g., Saccaromyces cerevisiae), insect (e.g., baculovirus), mammalian (e.g., Chinese hamster ovary), plant (e.g., safflower), and cell-free systems (e.g., rabbit reticulocyte).

As used herein, the term “affinity tag” refers to a short amino acid sequence, engineered into the sequence of a recombinant protein, to make its purification easier. Examples of the affinity tags which are known to the skilled person include but are not limited to (His)₆ or polyhistidine, Myc, FLAG, hemagglutinin, glutathione-S-transferase (GST), and a maltose-binding protein (MBP) tag. These protein tags can be located N-terninally, C-terminally and/or internally. In particular, the terms “his tag” and “polyhistidine tag” refer to the linear array of six histidine residues added to the amino or carboxy terminus of a recombinant protein, in order to easily purify the recombinant protein via metal affinity chromatography using a nickel-chelating resin or by use of polyhistidine-specific antibodies.

The terms “sample” and “specimen” in the present specification and claims are used in their broadest sense, and are meant to include a specimen or culture. These terms encompasses all types of samples obtained from humans and other mammals, including but not limited to body fluids such as urine, blood, fecal matter, cerebrospinal fluid (CSF), semen, saliva, and wound exudates, as well as solid tissue. However, these examples are not to be construed as limiting the sample types applicable to the present invention.

As used herein, the term “patient” and “subject” refer to a mammal who is a candidate for receiving medical treatment. In some embodiments, the subject is an individual suspected of having cancer, or having experienced a traumatic brain injury, or stroke.

The term “pathology” refers to the anatomic and/or physiological deviations from the normal that constitute a disease.

In the present invention, “cancer” refers to a malignant tumor whose cells have the properties of endless replication, loss of contact inhibition, invasiveness and the ability to metastasize and whose result, generally, if left untreated, is fatal.

As used herein, the term “traumatic brain injury” refers to a physical wounding suffered by the central nervous system and is characterized by blood-brain-barrier breakdown, marked edema formation, gliosis, and neuronal necrosis.

In the present invention, “stroke” refers to a cerebrovascular accident characterized by a sudden loss of consciousness, often with resulting paralysis, caused by hemorrhage into the brain, either due to blockage of blood flow to the brain by an embolus or thrombus, or due to the rupture of an artery exterior to yet supplying the brain, causing a loss of blood supply to the brain.

The term “control” refers to subjects or samples which provide a basis for comparison for experimental subjects or samples. For instance, the use of control subjects or samples permits determinations to be made regarding the efficacy of experimental procedures. In some embodiments, the term “control subject” refers to animals, which receive a mock treatment.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that it is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

The terms “high stringency conditions” and “stringent conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH2PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5×SSPE, 0.1% SDS at 42° C. when a probe of about 500 nucleotides in length is employed.

As used herein, the term “Northern blot” refers to methods for transferring denatured RNA onto a solid support for use in a subsequent hybridization assay. Total RNA or polyA-enriched RNA is typically electrophoresed in an agarose gel, transfered to a membrane and probed with a radioactively-labeled DNA or RNA fragment to detect specific RNA sequences. Northern blots are routinely used in the art (See, e.g., Thomas, Proc Natl Acad Sci USA 77:5201-5205 [1980]; and Ausubel et al. (eds.), Current Protocols in Molecular Biology, John Wiley & Sons, Inc., New York [1994]).

The term “Southern blot,” as used herein, refers to methods for transferring denatured DNA, which has been fractionated by agarose gel electrophoresis, onto a solid support, for use in a subsequent hybridization assay. These methods typically entail the digestion of genomic DNA with a suitable restriction enzyme prior to agarose gel electrophoresis, transfer of the DNA to a membrane and incubation with a radioactively-labeled DNA or RNA fragment for detection of specific DNA sequences. Southern blots are routinely used in the art (See, Southern, J Mol Biol 98:503-517 [1975]; and Ausubel et al., supra [1994]).

As used herein, the term “polymerase chain reaction (PCR)” refers to a method for increasing the concentration of a segment of a target sequence in a DNA mixture without cloning or purification (See, e.g., U.S. Pat. Nos. 4,683,195, 4,683,202, and 4,965,188, hereby incorporated by reference). This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” When the template is RNA, a reverse transcription (RT) step is completed prior to the amplification cycles. Thus, this variation is termed “RT-PCR.”

The terms “primers” and “primer pair” as used herein, refers to relatively short pre-existing polynucleotide chains to which new deoxyribonucleotides can be added by a DNA polymerase.

As used herein the term “electrophoresis” refers to a method of separating molecules in a mixture (e.g., DNA or RNA fragments, or proteins). Specifically, an electric current is passed through a medium containing the mixture, and each molecule travels through the medium at a different rate, thereby causing the molecules to separate based upon their size and charge. Agarose gels are typically used for electrophoresis of nucleic acids, while polyacrylamide gels are commonly used for electrophoresis of proteins. “Agarose gels” are produced from a linear galactan purified from agar that forms a gel when it is heated and cooled. The term “ethidium bromide” as used herein, refers to a dye used to stain nucleic acids, which fluoresces when exposed to ultraviolet light.

The term “antibody” refers to polyclonal and monoclonal antibodies. Polyclonal antibodies which are formed in the animal as the result of an immunological reaction against a protein of interest or a fragment thereof, can then be readily isolated from the blood using well-known methods and purified by column chromatography, for example. Monoclonal antibodies can also be prepared using known methods (See, e.g., Winter and Milstein, Nature, 349, 293-299 [1991]). As used herein, the term “antibody” encompasses recombinantly prepared, and modified antibodies and antigen-binding fragments thereof, such as chimeric antibodies, humanized antibodies, multifunctional antibodies, bispecific or oligo-specific antibodies, single-stranded antibodies and F(ab) or F(ab)₂ fragments. The term “reactive” in used in reference to an antibody indicates that the antibody is capable of binding an antigen of interest. For example, a PAO-reactive antibody is an antibody that binds to PAO or to a fragment of PAO.

The term “specific binding” when used in reference to the interaction between an antibody and an antigen describes an interaction that is dependent upon the presence of a particular structure (i.e., the antigenic determinant or epitope) on the antigen. In other words, the antibody recognizes a protein structure unique to the antigen, rather than binding to all proteins in general (i.e., non-specific binding).

As used herein, the term “immunoassay” refers to any assay that uses at least one specific antibody for the detection or quantitation of an antigen. Immunoassays include, but are not limited to, Western blots, ELISAs, radio-immunoassays, immunofluorescence assays, immunohistochemistry and flow cytometry.

The terms “immunoblot” “Western blot” and “Western” refer to methods of detecting a specific protein or proteins in a complex protein mixture such as a cell extract or lysate. These methods, which are well known in the art (See, e.g., Towbin et al., Proc Natl Acad Sci USA 76:4350-4354 [1979]; and Ausubel et al. (eds.), Current Protocols in Molecular Biology, John Wiley & Sons, Inc., New York [1994]), involve fractionating the protein mixture by SDS-polyacrylamide gel electrophoresis, transferring the separated proteins onto a solid support such as nitrocellulose and detecting the protein(s) of interest by with an antibody. The bound primary antibody can be visualized by the use of a secondary antibody conjugated to an enzyme which produces a signal in the presence of a suitable substrate.

As used herein, the term “ELISA” refers to enzyme-linked immunosorbent assay. Numerous ELISA methods and applications are known in the art, and have already been described in detail elsewhere (See, e.g., Antibodies: A Laboratory Manual, Cold Spring Harbor Laboratory Press [1988]; and Ausubel et al. (eds.), Current Protocols in Molecular Biology, Ch. 11, John Wiley & Sons, Inc., New York [1994]). The term “ELISA” includes but is not limited to direct ELISA, indirect ELISA, sandwich ELISA and cell ELISA methods. For instance, a cell sample of interest is coated onto the bottom of a well of a 96-well microtiter plate, and under suitable conditions the sample is contacted with a PAO-reactive antibody, whose binding is then visualized by contact with a peroxidase-conjugated goat anti-mouse serum and a calorimetric substrate.

The terms “inimunofluorescent analysis” and “IFA” refer to microscopy methods in which a fluorescent-labelled antibody (e.g., a PAO-reactive monoclonal antibody conjugated to fluorescein) is used to detect the presence or determine the location of the corresponding antigen (e.g., hPAO in permeabilized hepatocytes) using a fluorescence microscope (e.g., microscope with ultraviolet light source).

As used herein, the term “immunohistochemistry” refers to microscopy methods in which location of an antigen of interest is visualized by using a labelled antibody as a detection agent. In some embodiments the microscope has a visible light source and the antibody of interest is labelled with an enzyme suitable for use in a calorimetric reaction.

The terms “flow cytometry” and “FACS analysis” refer to methods of measuring fluorescence from a suspension of stained cells (e.g., cells that have been placed in contact with a PAO-reactive antibody) flowing through a narrow orifice, usually in combination with one or two lasers to activate the dyes (e.g., fluorochrome bound to either primary or secondary antibody).

The term “reporter molecule” as used herein, refers to molecules such as enzymes and fluorochromes which are suitable for use as tools for detection of an antigen of interest (e.g., PAO). Appropriate reporter enzymes include but are not limited to enzymes that can be utilized in colorimetric reactions, including but not limited to alkaline phosphatase and horse-radish peroxidase. As used herein, the terms “fluorochrome” and “fluorescent dye” refer to compounds which have the ability to emit light of a certain wavelength when activated by light of another wavelength. In some embodiments, the term “fluorochrome” includes but is not limited to fluorescent compounds such as fluorescein, phycoerythrin, Texas red, and rhodamine.

As used herein, the term “kit” is used in reference to a combination of reagents and other materials which facilitate sample analysis. In some embodiments, the immunoassay kit of the present invention includes a suitable capture antibody, reporter antibody, antigen, detection reagents and amplifier system. Furthermore, in other embodiments, the kit includes, but is not limited to, components such as apparatus for sample collection, sample tubes, holders, trays, racks, dishes, plates, instructions to the kit user, solutions or other chemical reagents, and samples to be used for standardization, normalization, and/or control samples.

### DESCRIPTION OF THE INVENTION

This invention relates to compositions and methods for the treatment of pathologies associated with intracellular polyamine dysregulation. In particular, the present invention provides compositions and methods involving mammalian N¹-acetyl-polyamine oxidase (APAO or PAO) to treat cancer, cell damage, tissue damage caused by ischemia and reperfusion, inflammation, traumatic brain injury, stroke, and tissue developmental disorders. Methods for diagnosis and prognosis of cancer and other diseases are also provided by the present invention.

Functions of Polyamine Oxidase (PAO)

PAO has been implicated in many physiological functions. Inmnunoregulatory roles have been proposed for PAOs in pregnancy (Morgan and Illei, Br. Med. J. 280:1295-1297 [1980]), and in some autoimmune disease (Flescher et al., J. Clin. Invest. 83:1356-1362 [1989]). Oxidized polyamines have antimicrobial, antiviral (Bachrach, in Polyamines in Biology and Medicine, Morris and Marton (eds.) Marcel Dekker, New York, pp.151-168 [1981], antifungal (Levitz et al., Antonie van Leeuwenhoek 58:107-114 [1990]), and antiparasitic properties (Rzepczyk et al. Infect. Immun. 43:238-244 [1984]). Moreover, PAOs have been implicated in apoptotic processes in both animal (Parchmnet, in Polyamine in Cancer:Basic Mechanisms and Clinical Approaches, Nishioka (ed.) R. G. Landes, Austin pp. 99-150 [1996]) and plant cells.

The intracellular level of polyamines is strictly regulated by the modulation of the activities of enzymes that are involved in the pathways for polyamine biosynthesis and degradation, as well as systems involved in their transportation (Pegg, Biochem J. 234:249-62 [1986], Casero and Pegg, FASEB. J. 7:653-61 [1993]). The first enzyme for polyamine synthase is ornithine decarboxylase (ODC), which catalyses the conversion of ornithine into putrescine (Put). Subsequently Put is converted into spermidine (Spd) by Spd synthase, and Spd is converted into Spermine (Spm) by Spm synthase (Pegg, Biochem. J. 234:249-62 [1986]; Seiler et al., Biochem. J. 225:219-226 [1985]; and Tabor and Tabor, Annu. Rev. Biochem. 53:749-790 [1984]). Polyamine degradation occurs via a polyamine catabolic pathway that requires the spermine/spermidine N¹-acetyltransferase (SSAT) and PAO. In this pathway, Spm and Spd are acetylated first by SSAT in the cytoplasmic matrix (Pegg, Biochem. J. 234:249-62 [1986]; Pegg and McCann, Am. J. Physiol. 245:C212-221 [1981], van den Munckhofet al., J. Histochem. Cytochem. 43:1155-1162 [1995]). Next, N¹-acetyl-Spm and N¹-acetyl-Spd are oxidized by PAO to Spd and Put, respectively, within peroxisomes (van den Munckhof et al., supra [1995]). Thus, PAO is an integral part of the polyamine interconversion cycle, a major regulatory system that contributes to maintaining polyamine homeostasis of animal cells (Cipolla et al., Urol. Res. 24:93-98 [1996]). PAO activity is rather high when compared with activities of the biosynthetic enzymes. Thus, PAO is deemed important in regulating tissue levels of the polyamine (Shipe et al., CRC Critical Rev. in Clinical Lab Science [1981]). Also as part of the catabolic pathway, spermine oxidase (Wang et al., supra [2001]; Murray-Stewart et al., supra [2002]; Vujcic, et al., supra [2002]; and in International Patent Application No. WO 02/100884) converts Spm to Spd. PAO can also convert Spm to Spd, albeit inefficiently.

The elevated levels of N¹-acetylated polyamines observed in a variety of tumors, is associated with reduced PAO activity, suggesting that a product of polyamine oxidation may regulate cell replication (Morgan, Biochem. Soc. Trans. 13:322-326 [1985]; Bachrach, supra [1981]). For instance, the loss of PAO activity is directly related to the size and grade of human breast tumors (Wallace et al., Clin. Cancer Res. 6:3657-3661 [2000]), as the N¹-acetyl-Spm:Spd ratio gradually increases from stage I to stage IV breast cancer tumors (Lee et al., Cancer Lett. 133:47-56 [1998]). Similarly, the N¹-acetylated polyamine levels are much higher and PAO activity much lower in human colorectal cancer tumors than in neighboring tissue (Linsalata, Cavallini and Di Leo, Anticanc. Res. 17:3757-3760 [1997]). Numerous other studies further demonstrate the intimate involvement of PAO in cancer (See, e.g., Hu and Pegg, Biochem. J. 328:307-316 [1997]; Kramer et al., Cancer Res. 59:1278-1286 [1999]; Bergeron et al., J. Med. Chem. 43:224-235 [2000]; Mank-Seymour et al., supra [1998]; Lindsay, and Wallace, supra [1999]; and Chopra and Wallace, supra [1998]).

An unidentified human tumor-suppressor gene has been localized to the same region of Chromosome 10 as the hpao gene (Kim et al., Oncogene 17:1749-1753 [1998]; Albarosa et al., Genomics 41:345-9 [1997]; Chernova et al., Oncogene 20:5378-5392 [2001]; and Lee et al., Mamm. Genome 12:157-162 [2001]). Deletions or disruptions in this region have been found in small cell lung cancer (Kim et al., supra [1998]), bladder cancer (Kagan et al., Oncogene 16:909-913 [1998]), prostate cancer (Komiya et al., Genes, Chromosomes & Cancer 17:245-253 [1996]), brain tumors (Albarosa et al., supra [1997]; and Lee et al., supra [2001]), and glioblastoma cells (Chernova et al., supra [2001]).

The discovery of gene deletion and transcriptional variants of a human PAO gene, during the development of the present invention, indicate that PAO plays a fundamental role in controlling cell growth. Thus, hpao is contemplated to be a tumor-suppressor gene.

Molecular Biology and Biochemistry of PAO

PAO has been purified from rat, porcine and bovine liver (Seiler, supra [1995]; and Gasparian and Nalbandian, Biokhimiia 55:1632-1637 [1990]). PAO is a 56 kDa monomer, containing noncovalently bound FAD as the essential redox cofactor. Mammalian PAO has a narrow specificity for natural substrates (Spm, N¹,N¹²-acetyl-Spm, N¹-acetyl-Spm and N¹-acetyl-Spd). A few irreversible inhibitors have been identified including MDL 72521 and MDL 72527 (Seiler, supra [1995]). PAO inactivates the anticonvulsant, milacemide [2-(n-pentylamino)acetamide], which is also a time-dependent irreversible inactivator of mitochondrial MAO-B (O'Brian et al., Biochem. Pharmacol. 47:617-623 [1994]). In addition, PAO oxidizes the antimalariavantiparasitic agent MDL 27695 (N,N-bis{3-[(phenylmethyl)amino]propyl}-1,7-diaminoheptane) to an inactive form (Edwards et al., J. Med. Chem. 34:569-574 [1991]). Clearly the action of PAO may reduce the efficacy of other drugs. Thus, it is contemplated that a thorough understanding of the biochemical and kinetic properties of PAO provides the means for the design of drugs that are less prone to inactivation by this oxidase.

Similar flavoproteins (e.g., glutathione reductase and the flavoprotein subunit of the flavocytochrome c sulfite reductase) have been used to model the structure of MAO-A (Wouters and Baudoux, Proteins: Struct. Funct. Genet. 32:97-110 [1998]), or to select amino acyl residues of MAO-A and MAO-B for site-specific mutagenesis (Zhou et al., J. Biol. Chem. 273:14862-14868 [1998]; and Kirksey et al., Biochemistry 37:12360-12366 [1998]). However, prior to the development of the present invention, only the structures of the corn PAO and MAO-B were known (Binda et al., supra [1999]; and Binda et al., supra [2002]). Thus, little biochemical and structural information was available for PAO, prior to the development of the present invention.

In addition to the mammalian peroxisomal N¹-acetyl-polyamine oxidase genes described herein, the gene sequences of several forms of PAO are known; a so-called N¹-acetyl-Spd oxidase from Candida boidinii peroxisomes (Nishikawa et al., FEBS Lett. 476:150-154 [2000]) a Spm/Spd oxidase from corn (Tavladoraki et al., FEBS Lett. 426: 62-66 [1998]), a cytosolic human Spm oxidase (Wang et al., Cancer Res. 61:5370-5373 [2001]; Murray-Stewart, et al., Biochem. J. 368:673-677 [2002]). and a cytosolic murine Spm oxidase (GenBank Accession No. BC004831; Vujcic, et al., Biochem. J. 367:665-675 [2002]). The translated protein sequences of all of these amine oxidases have similarities with the bovine PAO (bPAO), murine PAO (mPAO) and human (hPAO) protein sequences described herein. In particular, they all have an easily identifiable FAD-binding motif near their N-termini. This, and other features of the primary structures, indicate that these enzymes are members of a larger class of amine oxidases, which includes: mitochondrial MAO-A and MAO-B, MAO-N from Aspergillus niger (S chilling and Lerch, Biochim. Biophys. Acta 1243:529-537 [1995]; and Sablin et al., Eur. J. Biochem. 253:270-279 [1998]), putrescine oxidase from Micrococcus rubens (Ishizuka et al., J. Gen. Microbiol. 139:425-432 [1993]), and tyramine oxidase from Micrococcus luteus (Roh et al., Biochem. Biophys. Res. Commun. 268:293-297 [2000]). Other evidence indicates that these oxidases belong to an even larger class (e.g., superclass) of enzymes called the GR₂ (glutathione reductase) superclass of flavoproteins (Dailey and Dailey, J. Biol. Chem. 273:13658-13662 [1998]; Dym and Eisenberg, Protein Sci 10:1712-1728 [2001]). This superclass includes glucose oxidase, cholesterol oxidase, D-amino acid oxidase, sarcosine oxidase, p-hydroybenzoate hydroxylase, phenol hydroxylase, fumarate reductase, succinate dehydrogenase, glutathione reductase, protoporphyrinogen oxidase and phytoene desaturase.

As detailed below in the Examples, the genes for PAO from humans, mice and cattle were cloned during development of the present invention. Moreover, PAO expression in normal mouse and human tissues was examined. PAO expression was also assessed in the context of apoptosis induced by N¹-acetyl-Spm treatment of cultured cells. In addition, recombinant human and murine PAOs were produced in bacterial expression systems. With the known corn PAO structure as the template (Binda, et al., supra [1999]), the mPAO amino acid sequence was used to homology model the structure of this mammalian oxidase.

Role of PAO in Development

Recycling of Put by intracellular PAO may be important for maturing animals. PAO is scarce in the brain and liver of newborn rats, but increases dramatically during postnatal development (Seiler, supra [1995]). Put does not easily pass through the blood brain barrier, and its half-life in the brain is longer than in other tissues. This effectively isolates the brain's polyamine pool from the rest of the body. Thus, PAO may be of paramount importance for the maintenance of the polyamine pool of the brain.

In adult rats, long-term inhibition of PAO by MDL 72527 (N¹,N⁴-bis(2,3,-butadienyl)-butane-1,4-diamine) leads to elevated levels of the N¹-acetylated polyamines, reduced Put and Spd levels in tissues, and a rise in the blood Spm levels. However, PAO inhibition had no apparent adverse effect on the MDL 72527-treated rats. Normal, mature animals do not suffer from a loss of PAO-generated Put or a build-up of the N¹-acetylated polyamines, as these forms are excreted from the cell. Compensation for lost Put can be achieved by an increased ODC activity, and Put can be acquired readily from the diet. Any excess N¹-acetylated polyamines are transported in the circulation to the kidney for excretion in urine. Apparently, under normal circumstances in mature animals, the primary role of PAO is for the maintenance of intracellular polyamine homeostasis (Morgan, Biochem. Soc. Trans. 26:586-591 [1998]; and Seiler, supra [1995]).

Role of PAO in Cancer Growth

While very high intracellular levels of Put and the polyamines are toxic to normal cells, cancer cells have an increased demand for these substances. MDL 72527 inhibition of PAO in tumor cells decreased the Put levels of the cells and slowed tumor growth. This drug was a more effective antitumor agent when used in conjunction with the potent ODC inhibitor 2-(difluoro-methyl)ornithine (Seiler, supra [1995]). Polyamine depletion is known to stimulate the immune system, therefore, a direct immune response may also contribute to the observed arrest in tumor growth.

Anti-cancer drugs cause increased expression of SSAT, which depletes cancer cells of Spm and Spd. N¹,N¹¹-bis(ethyl)norspermine down-regulates the polyamine biosynthetic enzymes ODC and S-adenosylmethionine decarboxylase, but dramatically up-regulates SSAT production (Hu and Pegg, Biochem. J. 328:307-316 [1997]; and Kramer et al., Cancer Res. 59:1278-1286 [1999]). In Chinese hamster ovary (CHO) cells, N¹,N¹¹-bis(ethyl)norspermine induced apoptosis. However, addition of the specific PAO inhibitor, MDL 72521 (N¹-methyl-N⁴-(2,3-butadienyl)-butane-1,4-diamine), prevented apoptosis (Hu and Pegg, supra [1997]). NCI H727 human female carcinoid cells have viable ssat genes on both X chromosomes, in contrast to normal cells where only a single ssat allele is translated into active SSAT. Addition of the antitumor polyamine analog, N¹,N¹²-bis(ethyl)spermine BESpm), to a H727 cell culture led to high levels of SSAT mRNA and SSAT activity. Apparently, an inappropriate expression of both ssat genes caused the higher sensitivity of the H727 tumor cells to the anticancer drug, BESpm (Mank-Seymour et al., supra [1998]). In both CHO cells and in female carcinoma cells, the hyper-expression of SSAT increased the production of N¹-acetylated polyamines. The elevated N¹-acetylated polyamine level in turn, resulted in higher PAO activity and apoptosis, indicating that PAO-generated hydrogen peroxide and possibly pre-cytotoxic 3-acetamidopropanal play important roles in apoptosis.

Etoposide, a topoisomerase II inhibitor, induced apoptosis in HL-60 human promyelogenous leukemia cells (Lindsay and Wallace, Biochem. J. 337:83-87 [1999]). While it was shown that alterations in polyamine oxidation did not initiate apoptosis, it was suggested that a PAO/SSAT-dependent cell death-generating cycle was slowly set in motion, peaking at approximately 48 hours. In particular, PAO oxidized N¹-acetylated polyamines to produce hydrogen peroxide, stimulating SSAT activity (Chopra and Wallace, Biochem. Pharmacol. 55:1119-1123 [1998]). Increased SSAT activity lead to increased levels of the N¹-acetylated polyamines, resulting in higher PAO activity and increased hydrogen peroxide and 3-acetamidopropanal production.

Role of PAO in Traumatic Brain Injury and Cerebral Ischemia/Reperfusion

Cerebral trauma escalated polyamine oxidation via PAO, exacerbating the injury. After inducing transient cerebral ischemia in rats, administration of the PAO inhibitor MDL 72527 lowered the reperfusion injury volume (Dogan et al., J. Neurosurg. 90:1078-1082 [1999]). Similarly, rats inflicted with traumatic brain injury had less brain edema formation and delayed cellular damage when given MDL 72527 (Dogan et al., J. Neurochem. 72:765-770 [1999]). PAO-generated 3-aminopropanal, rather than hydrogen peroxide, was identified as the culprit in ischemic neuronal and glial cell death (Ivanova et al., J. Exp. Med. 188:327-340 [1998]; Ivanova et al., Pro. Natl. Acad. Sci. USA 99:5579-5584 [2002]). In fact, 3-aminopropanal is cytotoxic to cultured glial cells, fibroblasts, endothelial cells, and various transformed cell lines. There is also evidence to suggest that 3-aminopropanal plays a role in apoptosis during murine embryonic limb bud formation, and that it may be involved in tumor necrosis (Ivanova et al., supra [1998]; and Parchment and Pierce, Cancer Res. 49:6680-6686 [1989]).

PAO Knock Out Animals and Transgenic Animals Expressing Exogenous PAO Genes, Mutants, and Variants Thereof

The present invention contemplates the generation of PAO-gene knock out animals, and transgenic animals comprising an exogenous PAO gene or homologs, mutants, or variants thereof. In preferred embodiments, the knock out and transgenic animals display an altered phenotype (e.g., dysregulation of polyamine metabolism) as compared to wild-type animals. In some embodiments, the altered phenotype is the overexpression of mRNA for a PAO gene as compared to wild-type levels of PAO expression. In other embodiments, the altered phenotype is the decreased expression of mRNA for an endogenous PAO gene as compared to wild-type levels of endogenous PAO expression. Methods for analyzing the presence or absence of such phenotypes include Northern blotting, mRNA protection assays, and RT-PCR. In other embodiments, the transgenic animals have a knock out mutation of the PAO gene. In still further embodiments, the transgenic animals express a PAO variant or a truncated PAO.

The transgenic animals of the present invention find use in dietary and drug screens. In some embodiments, the transgenic animals are fed test or control diets and the response of the animals to the diets is evaluated. In other embodiments, test compounds (e.g., a drug that is suspected of being useful to treat diseases contemplated to be associated with dysregulation of polyamine metabolism such as cancer) and control compounds (e.g., a placebo) are administered to the transgenic and control animals, and the effects are then evaluated.

The transgenic animals can be generated via a variety of methods. In some embodiments, embryonal cells at various developmental stages are used to introduce transgenes into a normal animal orpao-knock out animal, for the production of transgenic animals. Different methods are used depending on the stage of development of the embryonal cell. The zygote is the best target for micro-injection. In the mouse, the male pronucleus reaches the size of approximately 20 micrometers in diameter which allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the foinder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a normal or pao-knockout non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad Sci. USA 82:6927 [1985]). Transfection is easily and efficiently obtained by culturing the blastomeres on a monolayer of virus-producing cells (Van der Putten, supra; Stewart, et al., EMBO J., 6:383 [1987]). Alternatively, infection can be performed at a later stage. Virus or virus-producing cells can be injected into the blastocoele (Jahner et al., Nature 298:623 [1982]). Most of the founders will be mosaic for the transgene since incorporation occurs only in a subset of cells which form the transgenic animal. Further, the founder may contain various retroviral insertions of the transgene at different positions in the genome which generally will segregate in the offspring. In addition, it is also possible to introduce transgenes into the germiline, albeit with low efficiency, by intrauterine retroviral infection of the midgestation embryo (Jahner et al., supra [1982]). Additional means of using retroviruses or retroviral vectors to create transgenic animals known to the art involves the micro-injection of retroviral particles or mitomycin C-treated cells producing retrovirus into the perivitelline space of fertilized eggs or early embryos (PCT International Application No. WO 90/08832 [1990], and Haskell and Bowen, Mol. Reprod. Dev., 40:386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells which have integrated the transgene. This technique obviates the need for growth of the transfected ES cells under appropriate selective conditions prior to transfer into the blastocoel.

In still other embodiments, homologous recombination is utilized to knock-out gene function or to create deletion mutants (e.g., mutants in which various PAO domains are deleted). Methods for homologous recombination are described in U.S. Pat. No. 5,614,396, incorporated herein by reference.

Drug Screening Using PAO

The present invention also provides methods and compositions for using PAO as a target for screening drugs that can alter polyamine metabolism.

One technique for drug screening provides high throughput methods for identifying compounds having suitable binding affinity to PAO peptides and is described in detail in WO 84/03564, incorporated herein by reference. Briefly, large numbers of different small peptide test compounds are synthesized on a solid substrate, such as plastic pins or some other surface. The peptide test compounds are then reacted with PAO peptides and washed. Bound PAO peptides are then detected by methods well known in the art.

Another technique uses PAO antibodies, generated using methods known in the art. Such antibodies capable of specifically binding to PAO peptides compete with a test compound for binding to PAO. In this manner, the antibodies can be used to detect the presence of any peptide that shares one or more antigenic determinants of the PAO peptide.

In some embodiments of the present invention, compounds are screened for their ability to inhibit the binding of a labeled substrate to PAO. Any suitable screening assay may be utilized, including, but not limited to, those described herein. The present invention also contemplates many other means of screening compounds. The examples provided above are presented merely to illustrate a range of techniques available. One of ordinary skill in the art will readily appreciate that many other screening methods can be used.

In particular, the present invention contemplates the use of cell lines transfected with PAO and variants or mutants thereof for screening compounds for activity, and in particular to high throughput screening of compounds from combinatorial libraries (e.g., libraries containing greater than 10⁴ compounds). The cell lines of the present invention can be used in a variety of screening methods. In some embodiments, the cells can be used in reporter gene assays that monitor cellular responses at the transcription/translation level. In still further embodiments, the cells can be used in cell proliferation assays to monitor the overall growth/no growth response of cells to external stimuli.

In some assays, the host cells are preferably transfected as described above with vectors encoding PAO or variants or mutants thereof. The host cells are then treated with a compound or plurality of compounds (e.g., from a combinatorial library) and assayed for the presence or absence of a response. It is contemplated that at least some of the compounds in the combinatorial library can serve as agonists, antagonists, activators, or inhibitors of the protein or proteins encoded by the vectors. It is also contemplated that at least some of the compounds in the combinatorial library can serve as agonists, antagonists, activators, or inhibitors of protein acting upstream or downstream of the protein encoded by the vector in a signal transduction pathway.

The cells are also useful in reporter gene assays. Reporter gene assays involve the use of host cells transfected with vectors encoding a nucleic acid comprising transcriptional control elements of a target gene (i. e., a gene that controls the biological expression and function of a disease target) spliced to a coding sequence for a reporter gene. Therefore, activation of the target gene results in activation of the reporter gene product.

Pharmaceutical Compositions Containing PAO, Analogs and Inhibitors

The present invention further provides pharmaceutical compositions which may comprise all or portions of PAO polynucleotide sequences, PAO polypeptides, inhibitors or antagonists of PAO bioactivity, including antibodies, alone or in combination with at least one other agent, such as a stabilizing compound, and may be administered in any sterile, biocompatible pharmaceutical carrier, including, but not limited to, saline, buffered saline, dextrose, and water.

The methods of the present invention find use in treating diseases or altering physiological states. Peptides can be administered to the patient intravenously in a pharmaceutically acceptable carrier such as physiological saline. Standard methods for intracellular delivery of peptides can be used (e.g. delivery via liposome). Such methods are well known to those of ordinary skill in the art. The formulations of this invention are useful for parenteral administration, such as intravenous, subcutaneous, intramuscular, and intraperitoneal. Therapeutic administration of a polypeptide intracellularly can also be accomplished using gene therapy as described above.

As is well known in the medical arts, dosages for any one patient depends upon many factors, including the patient's size, body surface area, age, the particular compound to be administered, sex, time and route of administration, general health, and interaction with other drugs being concurrently administered.

Depending on the condition being treated, these pharmaceutical compositions may be formulated and administered systemically or locally. Suitable routes may, for example, include oral or transmucosal administration; as well as parenteral delivery, including intramuscular, subcutaneous, intrarnedullary, intrathecal, intraventricular, intravenous, intraperitoneal, or intranasal administration.

For injection, the pharmaceutical compositions of the invention may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

In other embodiments, the pharmaceutical compositions of the present invention can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated.

Pharmaceutical compositions suitable for use in the present invention include compositions wherein the active ingredients are contained in an effective amount to achieve the intended purpose. For example, an effective amount of PAO may be that amount that suppresses apoptosis. Determination of effective amounts is well within the capability of those skilled in the art, especially in light of the disclosure provided herein.

In addition to the active ingredients these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Compositions comprising a compound of the invention formulated in a pharmaceutical acceptable carrier may be prepared, placed in an appropriate container, and labeled for treatment of an indicated condition. For PAO polynucleotides, polypeptides or inhibitors, conditions indicated on the label may include treatment of conditions related to apoptosis.

For any compound used in the method of the invention, the therapeutically effective dose can be estimated initially from cell culture assays. Then, preferably, dosage can be formulated in animal models particularly murine models) to achieve a desirable circulating concentration range that adjusts PAO levels.

A therapeutically effective dose refers to that amount of PAO or PAO inhibitor which ameliorates symptoms of the disease state. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds which exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors which may be taken into account include the severity of the disease state; age, weight, and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature (See, U.S. Pat. Nos. 4,657,760; 5,206,344; or 5,225,212, all of which are herein incorporated by reference). Those skilled in the art will employ different formulations for PAO than for the inhibitors of PAO. Administration to the bone marrow may necessitate delivery in a manner different from intravenous injections.

Experimental

The following Examples serve to illustrate certain preferred embodiments of the present invention and are not to be construed as limiting the scope thereof. All chemicals were of reagent grade or better, and purchased from common vendors.

In the experimental disclosure below, the following abbreviations apply: FAD (flavin adenine dinucleotide); ODC (ornithine decarboxylase); SSAT (CoenzymeA: spermidine/spermine-N¹-acetyltransferase); PAO and pao (polyamine oxidase and its gene, respectively); bPAO and bpao (bovine PAO and its gene, respectively); mPAO and mpao (murine PAO and its gene, respectively); hPAO and hpao (human PAO and its gene, respectively); cPAO (corn/maize PAO); MAO (mitochondrial monoamine oxidase); Spm (spermine); Spd (spermidine); Put (putrescine); cDNA (complementary DNA); EST (expressed sequence tag); kb (kilobases); bp (basepairs); HPLC (high performance liquid chromatography); PDB (Protein Data Bank); PCR (polymerase chain reaction); RACE (rapid amplification of cDNA ends); SDS-PAGE (sodium dodecyl sulfate-polyacrylamide gel electrophoresis); MW (molecular weight); Da (daltons); kDa (kilodaltons); eq (equivalents); μ (micron); M (Molar); μM (micromolar); mM (millimolar); N (Normal); mol (moles); mmol (millimoles); μmol (micromoles); nmol (nanomoles); kg (kilograms); g (grams); mg (milligrams); μg (micrograms); ng (nanograms); L (liters); mL (milliliters); μL (microliters); cm (centimeters); mm (millimeters); μm (micrometers); nM (nanomolar); ε (extinction coefficient); ° C. (degrees Centigrade); OD (optical density); UV (ultraviolet); psi (pounds per square inch); hydroxyapatite (HAP); N-[2-hydroxyethyl]piperazine-N¹-[2-ethanesulfonic acid] (HEPES); isopropyl thio-β-D-galactopyranoside (IPTG); kan (kanamycin); LB (Luria Bertani); 3-(N-morpholino)propane (MOPS); PBS (phosphate buffered saline); hrs (hours); RT (room temperature); Avestin (Avestin Inc., Ottawa, Ontario, CANADA); BIO 101 (Qbiogene, Carlsbad, Calif.); Bio-Rad (Bio-Rad Laboratories, Hercules, Calif); Boehringer (Boehringer Manheim, Germany); Clontech (Clontech Laboratories, Palo Alto, Calif.); Hewlett Packard (Hewlett Packard, Palo Alto, Calif.); IMAGE (IMAGE Consortium, Lawrence Livermore National Laboratory, Livermore, Calif.); Incyte or Genome Systems (Incyte Genomics Inc., Palo Alto, Calif.); Invitrogen (Invitrogen Corporation, Carlsbad, Calif.); Amicon or Millipore (Millipore Corp., Bedford, Mass.); Novagen (Novagen, Inc., Madison, Wis.); Operon (Operon Technologies Inc., Alameda, Calif.); Origin (Origin Technology, Inc., Rockville, Md.); Pharmacia (Amersham Pharmacia Biotech Inc., Piscataway, N.J.); Qiagen (Qiagen Inc., Valencia, Calif.); Sigma (Sigma Aldrich Corporation, St. Louis, Mo.); Spectrum (Spectrum Software Associates, Chapel Hill, N.C.); and Stratagene (Stratagene, Inc., La Jolla, Calif.).

### EXAMPLE 1

### Purification of bPAO and Microsequencing

In this example, the purification and amino acid sequencing of bPAO peptides is described. Fresh bovine livers were obtained at a local slaughterhouse. Immediately after removal from the animal, they were covered with ice and were transported to the laboratory within 1 hr. Following a published procedure (Gasparian, Biokhimiia 60:1632-1636 [1995]), bPAO was purified from the fresh liver, or tissue that had been stored at −70° C. as 1-inch cubes. Approximately 1 mg of nearly pure bPAO was obtained from 1 kg of tissue. The enzyme was purified further on a 10% Tris-HCl SDS Ready Gel (Bio-Rad), then electro-transferred onto an Immobilon-P^{sQ} membrane (Millipore), and Coomassie blue-stained. The membrane was submitted to the Biomolecular Resource Center (University of California, San Francisco, Calif.) for N-terminal sequence analysis by an automated Edman degradation procedure. SDS-PAGE purified bPAO yielded the N-terminal amino acid sequence: EAEAPGRGPRVLVVGGGIAGL (SEQ ID NO:7). Purified bPAO was electrophoresed again as above, and the Coomassie-blue stained bPAO band was excised and subjected to an in-gel tryptic digest at the Protein Sequencing Center (State University of New York, Brooklyn, N.Y.). Two major peptides were purified and sequenced, yielding the internal amino acid sequences SEHSFGGVVEVGAHWIHGPS (SEQ ID NO:8) and LMTLWDPQAQWPEPR (SEQ ID NO:9). The segments of the bPAO amino acid sequence that correspond to the shared motif of the FAD-containing protein superfamily are underlined (Dailey and Dailey, J. Bio. Chem. 273:13658-13662 [1998]).

### EXAMPLE 2

### Cloning and Sequencing bPAO, mPAO, and hPAO

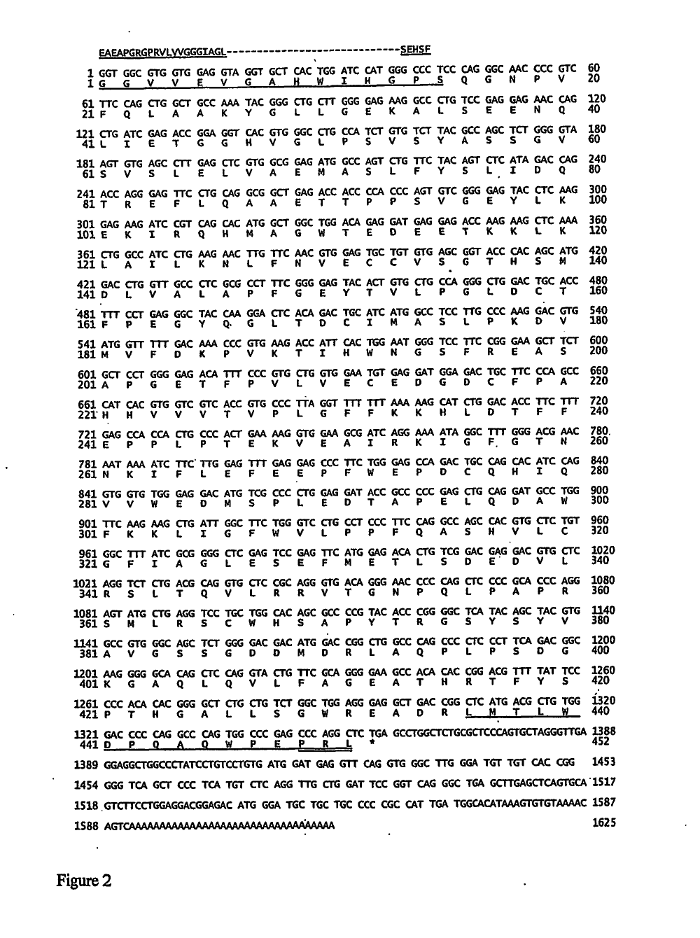

In this example, methods used to determine the mammalian PAO DNA sequences are described. The three bPAO peptide sequences described in Example 1, were used to search the GenBank and GenBank EST (expressed sequence tag) databases. Two mouse EST sequences that coded for an amino acid sequence 85% identical to that of SEQ ID NO:8 were identified (e.g., GenBank Accession Nos. AA437705 and AI098814, corresponding to IMAGE Nos. 819909 and 1482295, respectively). A bacterial strain (DH10B) containing the cDNA clone of GenBank Accession No. AA437705, which is flanked by SP6 and T7 promoters of the vector pSPORT1, was obtained from Incyte and sequenced. A 968 bp Xba I/Sal I fragment (mpao1) was excised from the purified plasmid, and labeled with ³²P-dATP (Pharmacia) by using a random primed DNA labeling kit (Boehringer). A λgt10 mouse 17 day embryo cDNA library (CLONTECH) with a titer of 1.1×10¹⁰ pfu/mL, and a Uni-ZAP XR bovine liver cDNA library (Stratagene) with a titer of 2.2×10¹⁰ pfu/mL were screened with the radiolabeled mpao1 probe using established methods (See, e.g., Sambrook et al., Molecular Cloning:A Laboratory Manual, second edition (Cold Spring Harbor Laboratory Press, New York) [1989]). About 2.7×10⁶ plaques for a bovine liver cDNA library, and about 2.5×10⁶ plaques for a mouse embryo cDNA library were screened. Seventeen positive bovine and sixteen positive mouse plaques isolated from the primary screening, were re-screened. The final positive clones contained mpao cDNA inserts of different lengths, as confirmed by Southern blotting (Sambrook et al., supra [1989]), using P³²-labeled mpao1 as a probe. Several positive plaques with large pao fragments were isolated, subcloned and sequenced. Excision of the pBluscript phagemid from the Uni-ZAP XR vector was carried out according to the manufacturer's instructions (Stratagene). The cDNA inserts contained in the pBluscript phagemids were sequenced using the T3 and T7 flanking primers. A 1.6 kb fragment of bpao (bpao1), coding for most of the C-terminal portion of bPAO, was isolated. The 5′-end of the fragment coded for a sequence that exactly matched the N-terminal portion of SEQ ID NO:8, while a region near the 3′-end coded for a protein sequence exactly matching the C-terminal end of SEQ ID NO:9, which was deduced to be the C-terminus of the enzyme. The partial bPAO cDNA (SEQ ID NO:1) and protein (SEQ ID NO:2) sequences are shown in FIG. 2.

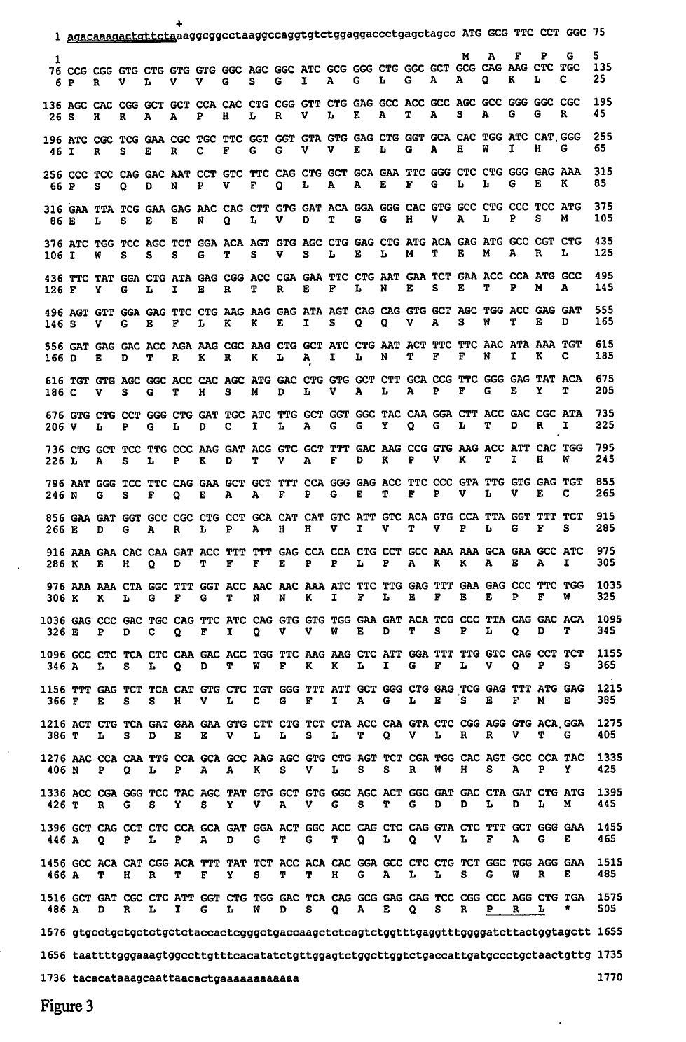

DNA isolated from one plaque of the mouse λgt10 library, was treated with Sal I and cloned into the Sal I sites of pUC19 to give the plasmid, pUC19-MPAO1, which was used for double-stranded sequencing. The missing 5′-segment of mpao was obtained by using the 5′-RACE PCR method using mouse 17-day embryo Marathon Ready cDNA (CLONTECH) as the template, and using a SMART RACE™ PCR cDNA Synthesis Kit (CLONTECH) for the PCR reactions. The primers used for 5′-RACE PCR include a mpao gene-specific antisense primer, mpao1R (Operon) spanning nucleotides 333 to 309 of mpao (5′-GTTCTCTTCC GATAATTCTT TCTCC-3′, SEQ ID NO:10), and the CLONTECH AP1 universal sense adaptor primer which is specific for the Marathon Ready cDNA template. The parameters used for PCR were as follows; 5 cycles for 30 sec at 94° C. and 3 min at 72° C., 5 cycles for 30 sec at 94° C. and 3 min at 70° C., and 30 cycles for 20 sec at 94° C. and 3 min at 68° C. Using a 50-fold dilution of the resulting PCR product as template, and AP2 (an AP1 nested primer) and mpao1R as primers, a second PCR reaction was carried out using the same conditions. The resulting ˜400 bp cDNA fragment, was isolated from an 1% agarose gel using the BIO 101 Gene Clean Kit, and sequenced. The PCR product, along with mpao1, yielded a 1.7 kb section of mpao (mpao2) coding for an amino acid sequence that is extremely similar to the predicted amino acid sequence of bpao1. The complete mPAO cDNA (SEQ ID NO:3) and protein (SEQ ID NO:4) sequences are shown in FIG. 3.

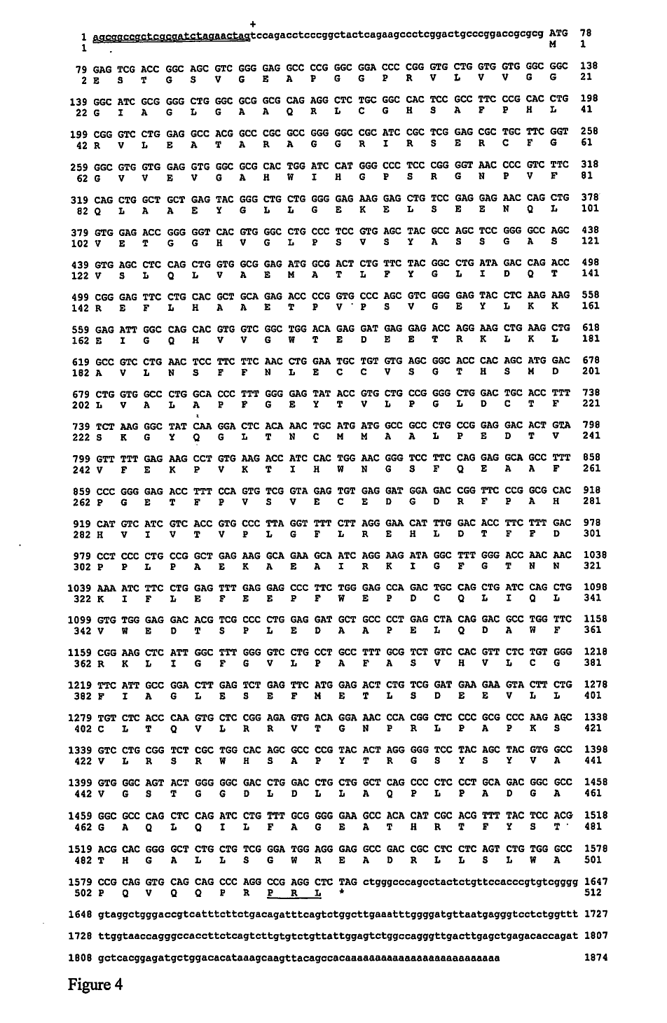

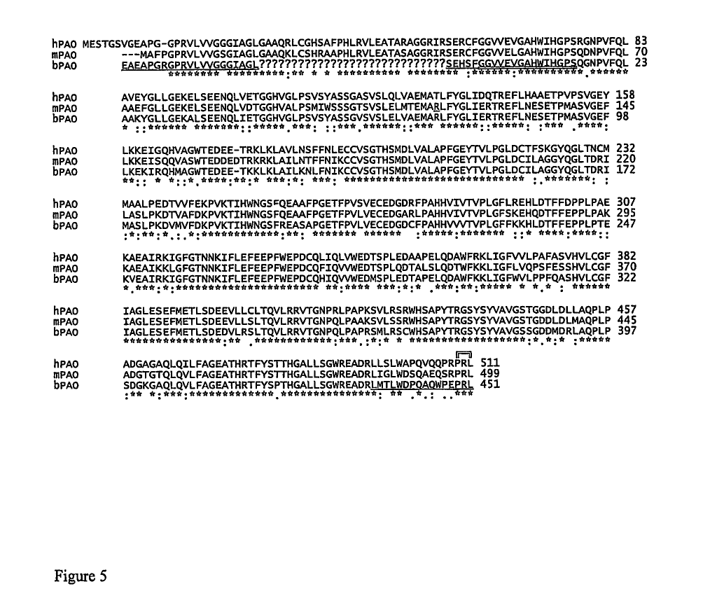

Subsequently, the mPAO cDNA sequence was used to screen the GenBank EST database for human entries. Several homologous human EST sequences were identified including an infant brain EST (GenBank Accession No. H05647). The cDNA clone corresponding to the human EST sequence was obtained from Genome Systems. The sequence derived from this human EST had an open reading frame of 319 amino acids. This C-terminal amino acid sequence was 83% identical to the aligned mPAO sequence. In order to isolate a larger portion of the human PAO cDNA, 5′-RACE PCR was used with a human liver cDNA library as a template (Clontech). This strategy was used successfully to obtain additional 5′ coding and 3′ non-coding hPAO sequence. The remaining N-terminal amino acid sequence was obtained from a second human EST (GenBank Accession No. AW662266). The complete hPAO cDNA (SEQ ID NO:5) and protein (SEQ ID NO:6) sequences are shown in FIG. 4, and an alignment of all three mammalian PAO protein sequences is shown in FIG. 5. The PRL motif at the C-terminus of the three mammalian PAO sequences is a putative peroxisomal transport sequence (Gould, J. Cell Bio. 108:1657-1664 [1989]).

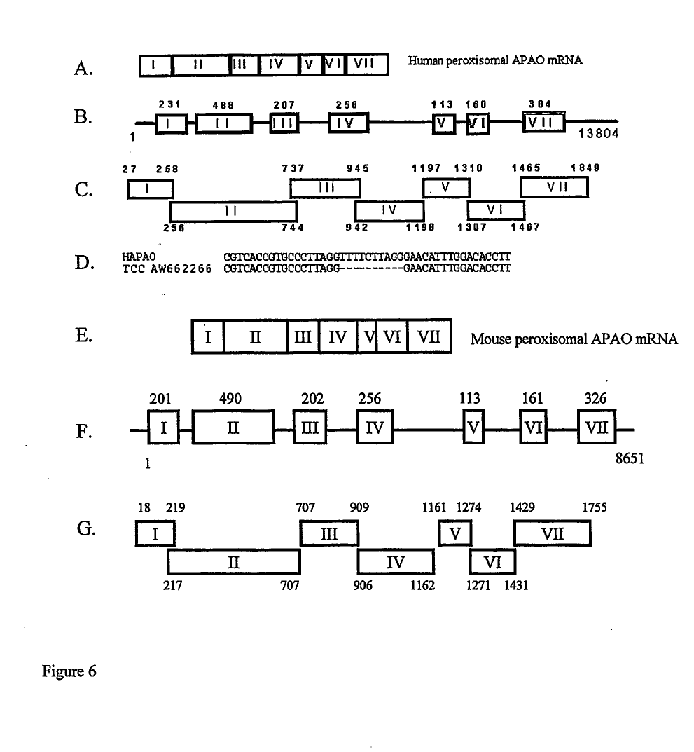

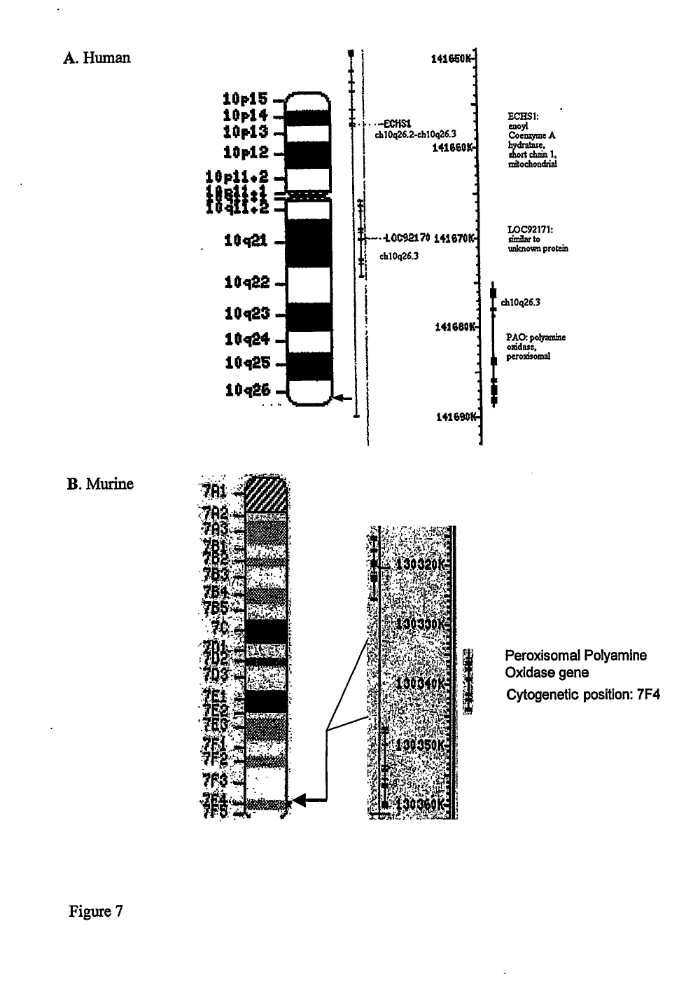

The hPAO cDNA sequence was used to BLAST search the human genome database from the National Center for Biotechnology Information website. The entire hpao sequence (contained in GenBank Accession No. AL360181.37) was found to map as 7 exons (See, FIG. 6 and SEQ ID NO:14, where base No. 2840 of this sequence corresponds to base No. 83941 of GenBank Accession No. AL360181.31). For SEQ ID NO:14, Exon I=bases 2840-3072, Exon II=bases 3574-4058, Exon III=bases 5033-5232, Exon IV=bases 7533-7785,Exon V=bases 12539-12641,Exon VI=bases 13163-13320,andExon VII=bases 14885-15266. The hpao gene mapped near the terminus of the q arm of chromosome 10, at cytogenetic map location ch10q26.3. (See, FIG. 7).

SEQ ID No: 14 also includes the promoter region (bases 1.2839) for hpao. A typical GC-rich region resides just upstream from the 5′-end of the Exon I. It contains nine consensus GC-boxes elements (2812 . . . 2799, 2611 . . . 2598, 2632 . . . 2619, 2692 . . . 2679, 2712 . . . 2699, 2732 . . . 2719, 2752 . . . 2739, 2782 . . . 2769, 2647 . . . 2634,), and another GC-box element can be seen further upstream (1983 . . . 1996). Within this GC-rich region, there are motifs for cap signal for transcription initiation (2877 . . . 2870, 2859 . . . 2866, 2842 . . . 2849) and stimulating protein 1 (2812 . . . 2800, 2632 . . . 2620, 2692 . . . 2680, 2712 . . . 2700, 2732 . . . 2720, 2752 . . . 2740, 2782 . . . 2770, 2647 . . . 2635, 2611 . . . 2595). Many other essential elements are spread out in this promoter region: retroviral TATA box (649 . . . 640), GATA-binding factor 3 sites (1803 . . . 1811, 987 . . . 995), GATA-binding factor 1 sites (986 . . . 995,17 . . . 26, 1802 . . . 1811, 2264 . . . 2273, 1643 . . . 1652, 534 . . . 525, 1149 . . . 1158, 200 . . . 191), GATA-binding factor 2 sites (1643 . . . 1652, 986 . . . 995, 1521 . . . 1512, 1613 . . . 1604), AP-4 (activator protein 4) binding sites (1128 . . . 1119, 1119 . . . 1128, 1584 . . . 1593, 1778 . . . 1787, 1865 . . . 1856, 282 . . . 273, 1602 . . . 1593), etc. Many other gene regulatory elements were also found in this promoter region: STREs (stress-response elements) (1692 . . . 1685, 2329 . . . 2322, 1037 . . . 1044, 345 . . . 352, 426 . . . 433, 2612 . . . 2605, 719 . . . 712, 2814 . . . 2807, 1717 . . . 1710), v-Myb (929 . . . 937, 246 . . . 1238), c-Myb (2109 . . . 2118, 2246 . . . 2255), tumor suppressor p53 (2090 . . . 2099, 578 . . . 569, 1566 . . . 1557). Thus, the human peroxisomal PAO gene is contemplated to be regulated by certain cellular conditions such as stress and tissue development.

The mPAO cDNA sequence was used to BLAST search GenBank. The entire mpao sequence is contained in GenBank Accession No. NW_{—}000335, where Exon I starts at base No. 1069500 (gene transcript ID No. XM 133921.1). The gene was found to map as 7 exons (See, FIG. 6 and SEQ ID NO:21). For SEQ ID NO:21, Exon I=bases 7477-7678, Exon II=8119-8609, Exon III=bases 9301-9503, Exon IV=bases 10810-11066, Exon V=bases 13441-13554, Exon VI=bases 14172-14332, and Exon VII=bases 15805-16129. The mpao gene mapped near the terminus of one arm of murine chromosome 7, at cytogenetic map location 7F4 (See, FIG. 7).

SEQ ID NO:21 also includes a promoter region (1 . . . 7476) upstream from the 5′-end of Exon I. It contains a typical GC-rich region with several GC-boxes (7476 . . . 6801, 7383 . . . 7370, 7465 . . . 7452, 6814 . . . 6801, 7441 . . . 7428), and a consensus motif required for promoters. The consensus motifs for the cap signal (transcription initiation) (7306 . . . 7313, 7393 . . . 7400, 7409 . . . 7416) and stimulating protein 1 (SP1) (7383 . . . 7371, 7465 . . . 7453) also reside in this region. Within this promoter region, other essential elements are also present: CCAAT/enhancer binding protein beta (4162 . . . 4149, 1539 . . . 1526, 2673 . . . 2686), GATA-binding factor 1(3487 . . . 3500, 4046 . . . 4033, 576 . . . 589), GATA-binding factor 2 (3489 . . . 3498, 7009 . . . 7000, 4454 . . . 4445, 6289 . . . 6298, 4817 . . . 4808), GATA-binding factor 3 (5451 . . . 5460, 5601 . . . 5592, 6041 . . . 6050, 4060 . . . 4051, 1439 . . . 1448, 5731 . . . 5740), cellular and viral TATA box elements (262 . . . 248, 2201 . . . 2215, 3587 . . . 3573, 4426 . . . 4440). In addition, there are c-Myb (44 . . . 35, 190 . . . 199, 1481 . . . 1472, 872 . . . 881, 2386 . . . 2377, 3265 . . . 3256), tumor suppressor p53 regulatory elements (622 . . . 612, 3331 . . . 332, 1789 . . . 1780, 1713 . . . 1722, 1521 . . . 1530) and STREs (3531 . . . 3538, 3494 . . . 3501, 2907 . . . 2900) motifs, which are contemplated to control mpao expression under certain conditions.

### EXAMPLE 3

### Production and Purification of Recombinant mPAO and hPAO

In this example, the methods used to produce and purify recombinant mPAO and hPAO in bacteria are described. The pET29 c(+) vector (Novagen) was used to construct a mpao prokaryotic expression system. E. coli DH5α was used throughout for plasmid subcloning. First, a 5′-end fragment was generated by PCR using mpao1 as the template for the gene-specific antisense primer mpao1R, and a sense primer containing Sac I and Nde I sites, and an ATG start codon 5′-GCGAGCTCAT ACATATGGCG TTCCCTGGCC CGCGG-3′ (SEQ ID NO:11), where the single underlines indicate the Sac I and Nde I restriction enzyme sites, respectively. The PCR product was restriction digested, and the resulting Sac I/Bam HI fragment was subcloned into pUC19-MPAO1 at Sac I and Bam HI sites to give pUC19-MPAO. Next, the full length cDNA of mpao was released by Nde I and Hind III treatment, and ligated into Nde I and Hind III sites of pET29c to give a plasmid denoted pET-MPAO. E. coli BL21 GOLD (DE3) (hvitrogen) was transformed with this plasmid for mPAO production.

The E. coli transformant was grown on Luria-Bertani (LB) agar plates that contained 30 μg/mL kanamycin. A single positive colony was inoculated into 3 ml of LB broth containing 30 μg/mL of kanamycin (LB-kan) for overnight growth at 37° C. Approximately 500 μl of this culture was then inoculated into 80 mL of fresh LB-kan medium and grown overnight. Five mL of the culture were transferred to each of five 2-L flasks containing 1 L of fresh LB-kan medium, and the cultures grown overnight at 37° C. with shaking. Each flask was used to inoculate five 12-L fermentors containing LB-kan media. Cell growth resumed at 30° C. with rapid stirring and vigorous aeration. When the cell density reached an OD₆₀₀=0.6-0.7, IPTG was added to a final concentration of 50 μM. Bacterial growth was allowed to continue until the cell density reached an OD₆₀₀=1.5-2.0. The cells were harvested by centrifugation. About 260 g of cell paste were obtained from 60 L of growth media, and stored at −70° C.

Selected fractions for the various steps in the following purification were assayed for N¹-acetyl-Spm oxidase activity by a modification of a published method (Holt et al., Anal. Biochem. 244:384-392 [1997]), which measures the time-dependent formation of H₂O₂. The assay stock solutions were: (A) 100 mM vanillic acid (in order to dissolve the acid, the pH of the solution was adjusted to 7 with KOH); (B) 50 mM 4-aminopyrine; (C) 400 units/mL of horse radish peroxidase; (D) 50 mM N¹-acetyl-Spm; and (E) 100 mM glycine/KOH, pH 9.5, a pH near which the maximal activity was reported to be attained (Hölttä, Methods Emzymol. 94:306-311 [1983]). Thirty μL each of solutions A through D were mixed with 2.86 mL of solution E, and 50 μL of this mixture was pipetted into individual wells of a 96-well plate. Anywhere from 1 to 50 μL of a particular fraction was added to a single well, and the relative activity of different fractions was assessed visually by the time-dependent intensity change of the developing pink color. The purity of various fractions was also determined by SDS-PAGE using pre-cast, 10-20% Tris-HCl Ready Gels (Bio-Rad), following the manufacturer's instructions.

The purification was initiated by placing 260 g of the frozen E. coli cell paste in a large beaker with 10 mM MOPS buffer, pH 7.25. The pH of the MOPS buffer was adjusted at 21° C. to yield an estimated pH of 7.35 at 4° C. (e.g., the temperature at which the purification was carried out unless noted otherwise). The total volume was approximately 800 mL. Once thawed, the cell paste was homogeneously suspended with a tissue grinder with a large glass/teflon piston (Potter/Elvehjem), after which the mixture was passed twice through a Avestin Emulsiflex C5 Homogenizer at 15-20,000 psi. At this point, about 15 mg of solid FAD was dissolved into the solution. The supernatant was centrifuged at 50,000×g, for 30 min and then dialyzed against 13 L of 10 mM MOPS buffer, pH 7.25 for 4 hr, and against 13 L of fresh 10 mM MOPS buffer, pH 7.25 overnight. The dialyzed solution was diluted to 2 L with the 10 mM MOPS buffer, pH 7.25, and applied, with a flow rate of ˜20 mL/min, to a 14×25 cm DEAE cellulose (Whatman, DE-53) column that had been equilibrated with this buffer. The column was washed with 2 L of the same buffer, and then a gradient elution from 0 to 400 mM KCl (8 L total gradient volume) in the MOPS buffer was initiated, at which time, the collection of 26-mL fractions was started. The flow rate was gradually increased from 20 to 29 mL/min from the start to the finish of the gradient elution. Significant activity was spread widely from tubes 180 to 300 (4.7 to 7.8 L), which were later combined. The volume was reduced to ˜500 mL using Amicon pressure concentrators fitted with Amicon YM-10 membranes. After dissolving ˜15 mg of solid FAD, the resulting solution was dialyzed for 4 hr against 13 L of 10 mM HEPES buffer, pH 7.8. The pH of the HEPES buffer was adjusted at 21° C. to yield an estimated pH of 8.05 at 4° C. The solution was then dialyzed overnight against 13 L of fresh HEPES buffer. The dialyzed sample was applied to a HEPES buffer-equilibrated 5×39 cm DEAE Spherodex LS column packed with 100-300 μm sized beads (Sepracor/IBF). The column was washed with 500 mL of the 10 mM HEPES buffer, before starting a 2.4 L gradient from 0 to 500 mM KCl, in the same buffer. The column, with a 7 ft pressure-head, was run at the maximum flow rate, and once the gradient was started, 26 mL fractions were collected. The majority of the activity eluted in tubes number 82-108, which were combined (˜700 mL). The sample was concentrated to ˜50 mL as described earlier, and then dialyzed for 4 hr, against 7 L of 10 mM KH₂PO₄/KOH buffer, pH 7.2, and then, overnight, against 7 L of fresh buffer.

The next step in the purification involved ion-exchange chromatography on an MONO P HR 5/20 column (Amersham/Pharmacia) at room temperature, using the following solutions: (I) H₂O and (II) 1 M KH₂PO₄ mixed with 1 M K₂HPO₄ to give 1 M potassium phosphate buffer, pH 7.2. After injecting 2 mL of the sample at a flow rate of 1 mL/min, proteins were eluted with the following gradient: 0% solution II at t=0; 0% to 1% II in 4 min; 1% to 30% II in 125 min. mPAO, which eluted from 38-41 min, was collected as a single fraction and immediately placed on ice. This step was repeated until the entire sample had been processed. The mPAO fractions from all the MONO P runs were combined, concentrated and washed into 1 mM KH₂PO₄/KOH buffer, pH 7.2, using eight 2 mL Centricon-10 concentrators (Amicon). After concentration, the final volume of the mPAO containing solution was 2 mL in the 1 mM buffer.

The final step in the purification involved chromatography on a 1×10 cm ceramic hydroxyapatite (HAP) column (e.g., type II HAP from Bio-Rad packed into an Amersham/Pharmacia HR 10/10 column) run at room temperature. The mPAO sample in 100 μL was diluted to 1 mL with H₂O, and the entire sample injected immediately onto the HAP column with a flow rate of 2 mL/min. The elution was carried out as follows: from t=0 to 7 min, 0% II; from t=7 to 9 min, 0 to 1% II; from t=9 to 19 min, hold at 1% II. mPAO eluted as a broad peak from t=14 to 17 min. This step was repeated until the entire sample from the MONO P column had been processed. The combined fractions were concentrated as for the MONO P fraction and washed into 10 mM KH₂PO₄/KOH buffer, pH 7.2, to give a 3.68 mg/mL mPAO solution, based on an ε₄₅₈=10,400 M⁻¹ cm⁻¹ and a MW=55,887 Da for the enzyme. The enzyme was judged to be pure by SDS-PAGE, and by ion-exchange chromatography on an analytical TSK DEAE 2SW column (0.4×25 cm; a 0.75 mL/min flow rate, with a gradient from 1% to 50% solution II in 30 min; a single sharp peak eluted at 23 min). The yield of pure mPAO was 36.8 mg.

Using the conditions for the steady-state kinetic assay described below, mPAO was determined to be stable at 2-4 mg/mL when frozen at −20° C. or −80° C. and thawed through several cycles. However, at a concentration of 30 μg/mL, activity was lost quickly after several freeze/thaw cycles, with more rapid loss occurring at −80° C. than at −20° C. When 33% (v/v) ethylene glycol was added, mPAO was stable for several cycles of freezing and thawing, for solutions containing 20 μg/mL to 4 mg/mL, regardless of the storage temperature. Thus, mPAO was stored at −20° C. in the presence of 33% (v/v) ethylene glycol. Ethylene glycol elimination and buffer exchange was accomplished easily by several concentration/dilution cycles using Centricon-10 centrifuge concentrators.

Similarly, a full-length hPAO cDNA fragment was cloned into the pTrcHisA vector Invitrogen), and used to transform E. coli DH5α for production of recombinant hPAO. The recombinant hPAO contained a poly-histidine tag at its N-terminus for purification purposes. A centrifuged bacterial cell extract obtained from a 2 L culture, was applied to a Nickel-NTA Superflow column (Qiagen). The Nickel column preferentially bound the His-tagged hPAO yielding several mg of pure hPAO. Recombinant His-tagged mPAO was also expressed to high levels with this system.

### EXAMPLE 4

### Spectral Characterization and Redox Properties of mPAO

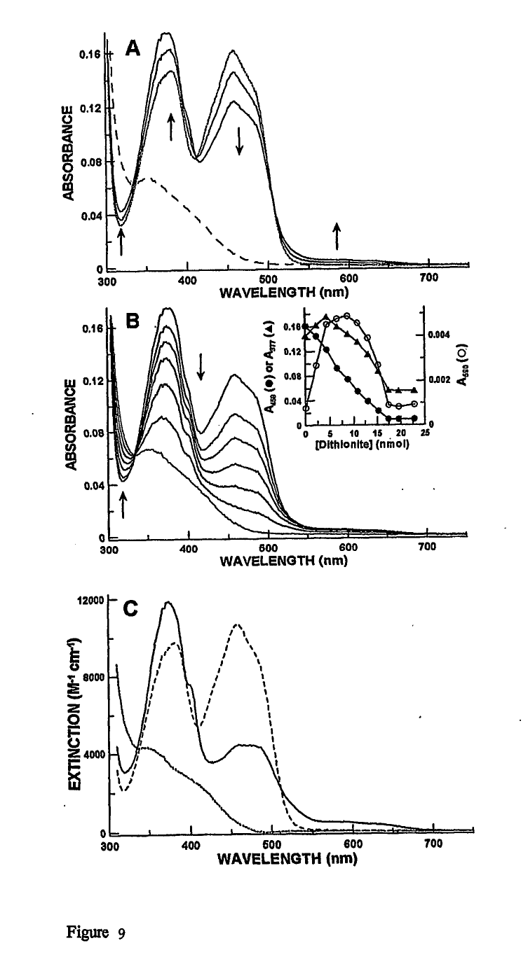

In this example the methods used for the anaerobic reductive titration of the purified recombinant MPAO are described. All UV-visible spectra were recorded with a Hewlett-Packard 8452A diode array spectrophotometer. mPAO, in 50 mM KH₂PO₄/KOH buffer, pH 7.6, at 21° C., was titrated anaerobically with a 0.541 μM sodium dithionite solution (See, FIG. 9). The dithionite solution was standardized by titrating anaerobically a FAD solution of known concentration (ε₄₄₅=11,300 M⁻¹cm⁻¹). The anaerobic cuvette and other details of this procedure are described elsewhere (Edmondson and Singer, J. Biol. Chem. 248:8144-8149 [1973]; Efimov et al., Biochemistry 40:2155-2166 [2001]; and Engst et al., Biochemistry 38:16620-16628 [1999]). The anaerobic mPAO solution also contained 50 mM D-glucose, 3 μg of catalase, and 50 μg of glucose oxidase to scavenge trace O₂. The spectral data was subjected to “Factor Analysis” using the Spectrum SPECFIT program (Spectrum Software Associates). The increase in absorbance in the 380 nm region indicates the formation of the red radical, while the small increases in the 550 to 700 nm region indicates the formation of a small amount of the blue radical (See, FIG. 9). The inset in FIG. 9, Panel B show a graph of A₃₇₇, A₄₅₈ and A₅₉₀ versus the amount of DT added. From this plot, it was determined that 15.2 mnol of DT were required to fully reduce the enzyme sample. These results provide valuable data regarding the interaction of a portion of the FAD molecule and an alpha-helix of mPAO. The extinction coefficients at 458 nm and 273 nm were found to be 10,600 and 99,200 M⁻¹ cm⁻¹, respectively. These values, when compared to theoretical values, confirm the presence of 1 molecule of FAD bound per molecule of PAO. The FAD was shown to be noncovalently bound by treatment of a solution of mPAO with 5% trichloracetic acid. The released flavin was quantitated by its fluorescence intensity relative to that of a standard solution of FMN (Singer and McIntire, Methods Enzymol. 106:369-378 [1984]).

The anaerobic titration of mPAO, in 50 mM KH₂PO₄/KOH buffer, pH 7.6, at 21° C., with a solution of N¹-acetyl-Spm is contemplated. For this titration, both the enzyme and substrate solutions contain D-glucose, catalase and glucose oxidase.

### EXAMPLE 5

### Steady-State Kinetic Analysis of mPAO

In this example the methods used to examine mPAO steady state kinetics are described. Spectrophotometric assays were done at 30° C. in a potassium phosphate buffer (e.g., 50 mM KH₂PO₄/KOH buffer, pH 7.6 saturated in air or in pure oxygen), using a published procedure which provides a continuous monitor of the H₂O₂ produced (Holt et al., Anal. Biochem. 244:384-392 [1997]). These assays were done in 1 mL cuvette with 0.8 mL of solution containing varying amounts of substrate, 1 mM vanillic acid, 0.5 mM 4-aminopyrine, 4 units of horseradish peroxidase, and 0.1-0.2 μg of mPAO. The reactions were monitored at 498 nm with a UVIKON 840 spectrophotometer (KONTRON Instruments) for the formation of the quinoneimine dye (ε₄₉₈=4,650 M⁻¹cm⁻¹ at pH 7.6), the condensation product of vanillic acid and oxidized 4-aminopyrine. The latter is produced from 4-aminopyrine by the action of horseradish peroxidase that has been oxidized by H₂O₂ (Holt et al., supra [1997]). The assays were done at 25° C. by varying the concentration of the amine substrate, while the oxygen concentration in the assay solution was constant at the air-saturating level of 237 μM. The data were fit, by nonlinear regression to steady-state kinetic equations (McIntire et al., Biochem. J. 228:325-335 [1985]), which provided the values, and standard deviations thereof, for k_{cat}′, K_{S}′ and k_{cat}′/K_{S}′ (=Q, the specificity constant).

$$\begin{matrix}v = \frac{{k}_{cat}^{′} [ S ]}{{Ks}^{′} + [ S ]} & Equation 1 \\ v = \frac{Q [ S ]}{1 + \frac{[ S ]}{{Ks}^{′}}} & Equation 2\end{matrix}$$

It was determined that Spm, Spd, and Put (but not benzylamine), which are either very poor or nonsubstrates, are inhibitors of the oxidation reaction of the best substrate, N¹-acetyl-Spm. The dissociation constants, K_{D}, for these and other amines were estimated in the following manner. It was assumed that the competition was competitive, since in the presence of the inhibitors, at a saturating N¹-acetyl-Spm concentration, the rate of the reaction was equal to the value of the true k_{cat}. It was also assumed that the mechanism is of the ping-pong type. Thus, the applicable steady-state equation is (Segal, Enzyme Kinetics, John Wiley & Sons, New York, pp. 606-625 [1975]):

$$v = \frac{{k}_{cat} [ S ]}{Ks ( 1 + \frac{[ I ]}{{K}_{I}} ) + [ S ] ( 1 + \frac{{K}_{0}}{[ {O}_{2} ]} )} = \frac{{k}_{cat}^{′} [ S ]}{{Ks}^{″} + [ S ]} \qquad Equation 3$$

On the left, k_{cat} and K_{S} are the steady-state parameters for N¹-acetyl-Spm (S), and I and K_{I} (=K_{D}), represent the inhibitor and its inhibition constant, respectively. K_{IO} and K_{O} are the inhibition constant and Michaelis constant for O₂, respectively. The equation can be converted to the expression on the right side when:

$${k}_{cat}^{′} = \frac{{k}_{cat}}{( 1 + \frac{{K}_{0}}{[ {O}_{2} ]} )} , \\ {Ks}^{″} = {Ks}^{′} ( 1 + \frac{[ I ]}{{K}_{I}} ) , \\ {Ks}^{′} = \frac{Ks}{( 1 + \frac{{K}_{0}}{[ {O}_{2} ]} )} \qquad Equations 4 a , b , c$$

The expressions for k_{cat}′ and K_{S}′ (at the fixed [O₂]=237 mM) are the same as those when the inhibitor is absent. Since the values of k_{cat}′ and K_{S}″ were predetermined, inhibition assays were carried out where [S]≈K_{S}″, which, in the absence of I, would give a rate, ν≈0.5×k_{cat}″. In these assay, for each inhibitor, [I] was adjusted so the apparent rate was somewhat less then 0.5 k_{cat}′. In this situation, after substitution and rearrangement, Equation 1 reduces to:

$${K}_{I} = \frac{[ I ]}{\frac{{k}_{cat}^{′}}{{v}^{′}}} - 2 \qquad Equation 5$$

Since [I] and k_{cat}′ are known, and ν is the measured rate in the presence of the inhibitor, the value of K_{I}′ can be estimated. It is assumed that these apparent K_{I}′ are good approximations of the true K_{I} (=K_{D}) values. K_{I}′ values were estimated at four different concentrations of each of the following inhibitors, benzylamine, Spm, Spd, Put, and N⁸-acetyl-Spd. The calculated K_{I}′ values at the four different concentrations of each inhibitor were within ˜20% of each other. The estimated K_{I}′ values provided in Table 1, are the averages of these four values. If the reactions of the polyamine substrates and O₂ with mPAO obeys a ping-pong type mechanism, the apparent values, k_{cat}′/K_{S}′, are equal to the true k_{cat}/K_{S} values.

Some assays (oxygraph assays) were carried out by directly monitoring O₂ consumption in air-saturated buffer ([O₂]=0.237 mM) or buffer saturated with pure O₂ at 30° C. ([O₂]=1.12 mM). The depletion of O₂ was detected with a Yellow Springs Instruments, Inc. Model 53 Oxygen Monitor equipped with a Clark electrode. The true k_{cat} and K_{O} values for the oxidation of N¹-acetyl-Spm and N¹-acetyl-Spd were determined by progress-curve analysis for reactions that were allowed to go to completion ([O₂]=0 at t=8). Following a published procedure (Goudar et al., Biochim. Biophys. Acta 1429:377-383 [1999]), the data were fitted to the integrated Mechaelis-Menton equation. The analyses were done using Maple VI (Windows 2000) software (Waterloo Maple, Inc.) running on a PC computer. Using the apparent K_{I}′ values (Table 1) as a guide, the saturating concentrations of N¹-acetyl-Spm and N¹-acetyl-Spd were made high enough (3.7 mM) so that product inhibition by Spd and Put, respectively, was insignificant at all times during the reaction. Inhibition by the H₂O₂, formed as a product of polyamine oxidation by mPAO, was assessed by addition of 2 μL of a 30 mg/mL (30,000 units/mg) solution of catalase, after [O₂] reached zero.

TABLE 1
Steady-State Kinetic Parameter for the Reaction
of Various Amines (S) and O₂ (O) with Pure mPAO

| | k_{cat}′ | K_{S}′ | k_{cat}′/K_{S}′ | K_{I}′ = K_{D} |
|---|---|---|---|---|
| Compound (S) | (sec⁻¹) | (μM) | (M⁻¹sec⁻¹) | (μM) |
| N¹-acetyl-Spm | 4.53 ± 0.05 | 1.78 ± 0.1 | (2.54 ± 0.01) × 10⁶ | NA^{a} |
| N¹-acetyl-Spd | 4.85 ± 0.03 | 36.8 ± 1.1 | (1.32 ± 0.03) × 10⁵ | ND^{a} |
| N⁸-acetyl-Spd | 0 | 0 | 0 | 70 |
| N¹,N¹²-diethyl-Spm | 0.415 ± 0.012 | 150 ± 10 | (2.77 ± 0.13) × 10³ | ND |
| N¹,N¹¹-diethyl-nor-Spm | 1.93 ± 0.03 | 157 ± 7 | (1.27 ± 0.03) × 10⁴ | ND |
| benzylamine | 0 | 0 | 0 | ∞ (infinity) |
| Spm | 0.175 ± 0.005 | 716 ± 33 | (2.47 ± 0.01) × 10² | 750 |
| Spd | 0 | 0 | 0 | 190 |
| Put | 0 | 0 | 0 | 1,000 |
| synthalin | 0 | 0 | 0 | 0.05 |
| N-(3-aminopropyl) 1,10 | 0 | 0 | 0 | 35 |
| decanediamine | | | | |

^{a}NA indicates not applicable, and ND indicates not determined.

TABLE 2
Steady-State Kinetic Parameter for the Reaction
of Various Amines (S) and O₂ (O) with Pure mPAO

| | k_{cat}^{a} | K_{S}(calc)^{b} | k_{cat}/K_{Q}^{a} | K_{Q}^{a} |
|---|---|---|---|---|
| Compound (S) | (sec⁻¹) | (μM) | (M⁻¹sec−¹) | (μM) |
| N¹-acetyl-Spm | 8.0 ± 0.8 | 3.1 ± 0.3 | (4.4 ± 0.4) × 10⁴ | 180 ± 20 |
| N¹-acetyl-Spd | 13 ± 1 | 83 ± 8 | (4.3 ± 0.4) × 10⁴ | 301 ± 30 |

^{a}The true k_{cat}, K_{O}, and k_{cat}/K_{O} values were determined by progress-curve analysis of O₂ consumption, in the presence of a saturating concentration of N¹-acetylated polyamine substrate. The concentration of the substrate was assumed to be high enough to overcome any inhibition by the polyamine product formed during the reaction. The buffer was saturated with pure O₂ (=1.12 mM). The errors were estimated to be about 10%.
^{b}The “true” K_{S} values calculated from the apparent values determined in air-saturated buffer ([O₂] = 0.237 mM]. The calculations were done using the equation K_{S} = K_{S}′(1 + K_{O}/[O₂]) (See equations 4).

The catalase immediately converted each mole of H₂O₂ to 0.5 mol of O₂ (since [O₂]=1.12 mM for pure O₂-saturated buffer, then [O₂]=0.56 mM immediately after catalase addition). In the presence of catalase, O₂ depletion should occur at 0.5× the rate as it does in the absence of catalase (unless H₂O₂ inhibition is operating). With catalase present, each molecule of O₂ will be converted eventually to H₂O (4-electron equivalents), rather than to H₂O₂ (2-electron equivalents). Thus, to get the proper rates, all post-catalase Δ[O₂]/Δt values were multiplied by 2 for the N¹-acetyl-Spm or N¹-acetyl-Spd case. It was found that each resulting [O₂] versus time progress curve was superimposable on the corresponding pre-catalase curve for [O₂]=0.56 to 0 mM. This indicated that H₂O₂ inhibition does not occur.

Thus, N¹-acetyl-Spm and N¹-acetyl-Spd were found to be good mPAO substrates and Spm was found to be a poor substrate. In addition, N¹,N¹²-diethyl-Spm (also known as N¹,N¹²-bis[ethyl]spermine or BE-Spm) and N¹,N¹¹-diethyl-nor-Spm (also known as N¹,N¹¹-bis[ethyl]norspermine or BEN-Spm), were also found to be very good substrates for mPAO. These polyamines have been used widely to study the physiological effects of polyamine metabolizing enzymes. They down-regulate polyamine biosynthetic enzymes, but dramatically up-regulate SSAT synthesis (Hu, and Pegg, supra [1997]; Kramer et al., supra [1999]). Thus, when these substances are provided to cells, the intracellular levels of N¹-acetyl-Spm and N¹-acetyl-Spd rise significantly due to SSAT hyperproduction. This results in an increase in the extracellular transport of the N¹-acetylated polyamines, as well as an increase in the level of cellular H₂O₂ and 3-acetamidopropanal, via their oxidation. Consequently, increased H₂O₂ levels induce an increase in SSAT production (Seiler, Neurochem. Res. 25:471-490 [2000]). The increased levels of H₂O₂ (and possibly the increased amount of 3-acetamidopropanal, which can be convert in the cell to cytotoxic, 3-aminopropanal; Houen et al., supra [1994]) induces apoptosis. This can be eliminated by treating the cells with the potent mechanism-based PAO inhibitor MDL 72527 (N¹,N⁴-bis(2,3-butadienyl)-butane-1,4-diamine). Induction of apoptosis by this mechanism maybe contributing to killing precancerous cells, and contributing to the damage cause by ischemia and reperfusion (Mank-Seymour et al., supra [1998]; Lindsay and Wallace, supra [1999]; Chopra and Wallace, supra [1998]; Ha et al., Proc. Natl. Acad. Sci (USA) 94:11557-11562 [1997]; Ferioli et al., Biochem. Pharm. 58:1907-1914 [1999]; Rao et al., J. Neurochem. 74:1106-1111 [2000]; Hatcher et al., Soc. Neurosci. Abstr. Vol. 26:Program No. 769-9 [2000]; and Zoli et al., Brain Res. Molec. Brain Res. 38:122-134 [1996]; Ivanova, et al., supra [1998]; Ivanova, et al., supra [2002]).

Unless the levels of N¹-acetyl-Spm and N¹-acetyl-Spd are high enough to suppress the oxidation of BEN-Spm or BE-Spm, their efficacy in inducing apoptosis may be diminished. This raises several considerations for designing anticancer drugs, drugs that can be used to minimize ischemic and reperfusion tissue damage, or drugs for developmental problems directed at altering polyamine metabolism. First, the efficacy of any potential pharmaceutical may be diminished if it can be oxidized efficiently by PAO. Alternatively, it is possible that the PAO-oxidized drug is the real therapeutic agent (e.g., that hyperinducing SSAT production). Additionally, a PAO-oxidized drug may be toxic, or its toxicity might be diminished by further PAO oxidation. Interestingly, BEN-Spm is currently in Phase II clinical trials (Bergeron et al., supra [2000]). However, an understanding of the mechanism(s) is not necessary in order to make or use the present invention.

N-(3-Aminopropyl)-1,10-diaminodecane is known to be a ligand for the polyamine domain of the N-methyl-D-aspartate (NMDA) receptor. This strong antagonist interacts with both the NMDA and Gly recognition site of the receptor (Yonada et al., Brain Res. 679:15-24 [1995]). As shown in Table 1, this polyamine analog is a good inhibitor of mPAO, with an apparent K_{I}′ of 35 μM. During the development of the present invention, it was found that this polyamine was not oxidized by mPAO, even though it has a terminal diaminopropyl group as does Spm. However, Spm, which has the same disposition of amino groups along its chain as N¹-acetyl-Spm, but lacks the terminal N-acetyl functionality, is a poor mPAO substrate. Additionally, the spacing of the amino groups along the straight chain of N-(3-aminopropyl)-1,10-diaminodecan is contemplated to lead to its improper alignment in the active site, for oxidation of the appropriate carbon center.

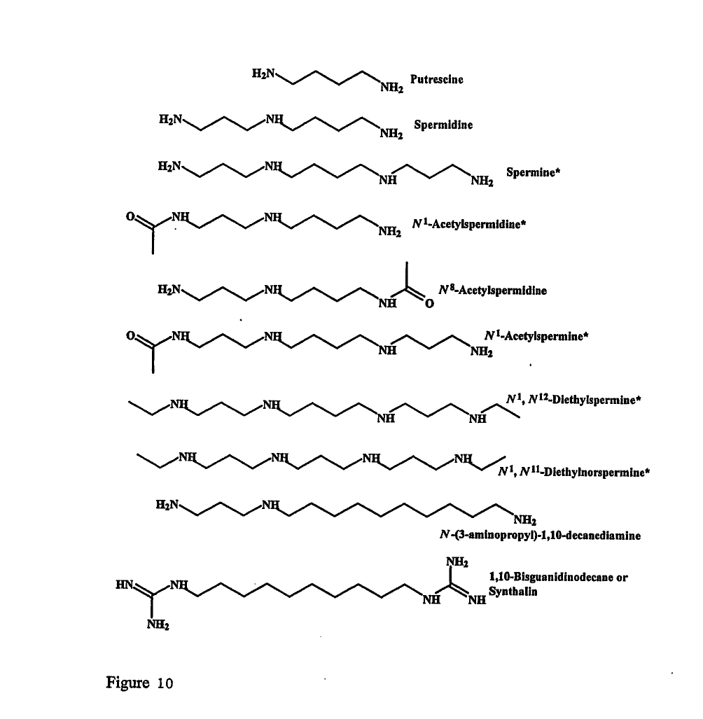

The spacing of amino groups suggests a reason why N⁸-acetyl-Spd is not oxidized by mPAO. It is suspected that this substrate, N¹-acetyl-Spm and N¹-acetyl-Spd all bind to mPAO with their N-acetyl group situated in the same binding site. This is contemplated to help align substrates correctly for oxidation. Thus, while the pertinent carbon center of the N-acetamidopropyl moieties (i.e., Ac-NH CH₂ CH₂*CH₂NH—] of N¹-acetyl-Spm and N¹-acetyl-Spd are properly position for oxidation by FAD, this carbon center is displaced by one methylene group in the N-acetamidobutyl moiety of N⁸-acetyl-Spd. As shown in FIG. 10, a non-oxidizable carbon center occupies the position favored by FAD (i.e., Ac-NHCH₂CH₂*CH₂CH₂NH—).

Synthalin (1,10-bisguanidinodecane), with an apparent K_{I}′ of 50 nM, is an extremely potent inhibitor of mPAO. Previously, this substance was thought to be only a potent potassium channel blocker and activator (Allard et al., FEBS Lett. 375:215-219 [1995]), and a noncompetitive antagonist of the NMDA receptor (Reynolds, J. Pharmacol. Exp. Ther. 263:632-638 [1992]; and Reynolds et al., J. Pharmacol. Exp. Ther. 259:626-632 [1991]). That synthalin is a much better inhibitor of mPAO than N-(3-aminopropyl)-1,10-diaminodecane is contemplated to be due to the spacing of the cationic guanidino groups along the straight chain molecule. For synthalin this spacing is contemplated to mimic the spacing of the terminal ammonium centers in N¹-acetyl-Spm and N¹-acetyl-Spd (See, FIG. 10). In fact, a terminal guanidino group of synthalin is contemplated to better imitate the acetamido group of N¹-acetyl-Spm or N¹-acetyl-Spd, than does an amino group as present in N-(3-aminopropyl)-1,10-diaminodecane.

In addition, one or more derivatives of synthalin (See, FIG. 10) with different number (n) of intervening methylene groups (n=10 for synthalin) between the guanidino groups are contemplated to be efficient inhibitors of PAO. Also, synthalin or a derivative thereof with one or both guanidino groups substituted with an amino group or an amidino group are contemplated to be effective PAO inhibitors. Furthermore, synthalin derviatives with alkylguanidino, alIylamidino, or alkylamino group(s) are contemplated to be effective inhibitors of PAO. Thus, some embodiments of the present invention provide synthalin variants defined by the following structure: R₁—(CH₂)ₙ—R₂, where both R₁ and R₂ represent guanidino, amidino, amino, alkylguanidino, alkylamidino, alkylamino groups or any mixed pairing of these groups (e.g., for synthalin, R₁ and R₁ are guanidino groups, and n equals 10 as shown in FIG. 10).

Amidino, amino, alkylguanidino, alkylamidino and alkyl amino groups are selected as suitable substitutes for the guanidino groups because, like the guanidino group, they are all positively charged functionalities. In fact, this property is contemplated to be important for efficient PAO inhibition. In the above formula, “n” is variable because there is expected to be an optimal methylene chain-length for effective PAO inhibition by different synthalin derivatives. Some of these derivatives are contemplated to possess high PAO inhibitory properties (e.g., as good or better than synthalin), but have reduced interactions with other systems. Synthalin derivatives with desirable features are identified using the methods disclosed herein.

### EXAMPLE 6

### Analysis of PAO Oxidation Products

In this example, methods are described for determining the nature of the products of substrate oxidation by PAO. In a solution at pH 7.6 containing catalase to destroy H₂O₂, mPAO and N¹-acetyl-Spm, at known concentrations, were allowed to react until the latter had been completely oxidized. mPAO and N¹-acetyl-Spd were similarly allowed to react. Both reactions solutions contained a known amount of N-(3-aminopropyl)-1,10-diaminodecane (Tocris Cookson) as an internal standard. This standard was found not to be a PAO substrate. Aliquots of these reaction mixtures were treated with dansyl chloride (5-dimethylamino-1-naphthalenesulfonyl chloride) obtained from Sigma. The resulting dansylated polyamine products from each reaction were analyzed by reverse-phase high-pressure liquid chromatography (HPLC) following established procedures (Hunter, Methods in Molecular Biology: Polyamine Protocols, Morgan (ed.) Human Press, Totowa, N.J., Vol. 79, Chapter 14, pp 119-123). A Prodigy HPLC column (octadecylsilyl silica gel, 5 micron particle size, 0.46×5.0 cm; Phenomenex) was used, with a flow rate of 1 mL/min, and the following elution gradient: 0 to 45% B from 0 to 0.1 min, 45 to 80% B 0.1 to 8 min, hold at 80% B from 8 to 11 min, 80 to 90% B from 11 to 12 min. Detection was accomplished with a Gilson Spetra/Glo fluorescence detector using a 7-51× excitation filter (330-400 nm) and a 3-72M emission filter (460-600 n m). Quantification and identification of the peaks was accomplished by dansyl chloride treatment and HPLC analysis of a standard solution containing known amounts of Spm, Spd, putrescine, N¹-acetyl-Spm, N¹-acetyl-Spd, and N-(3-aminopropyl)-1,10-diaminodecane (internal standard). The complete PAO oxidation of N¹-acetyl-Spm and N¹-acetyl-Spd was found to produce stoichiometric amounts of Spd and putrescine, respectively. This confirms that the PAO described herein is the classical mammalian peroxisomal N¹-acetyl-polyamine oxidase (Höltta, Biochemistry 16:91-100 [1977]; Bolkenius and Seiler, Int. J. Biochem. 13:287-292 [1981]; Höltta, Methods Enzymol. 94:306-311 [1983]; Seiler, Prog. Brain Res. 106:333-344 [1995]).

Aliquots of each of the above described enzyme-reaction solutions were treated with an equal volume of 2,4-dinitrophenylhydrazine (Acros Organics) solution (100 mg in 94 mL ethanol+6 mL of concentrated HCl). A standard solution containing known amounts of acrolein (Acros Organics), 3-acetamidcpropanal and 3-aminopropanal (infra) was treated in an identical manner. This treatment converts the three aldehydes to the corresponding 2,3-dinitronitrophenylhydrzone derivatives. The enzyme-reaction and standard solutions were analyzed by reverse-phase HPLC: Prodigy octadecylsilyl silica gel hplc column (supra): flow rate, 1 mL/min; gradient elution-0% B for 0.5 min, 0 to 35% B from 0.5 to 1.5 min, hold at 35% B from 1.5 to 5.0 min, 35 to 100% B from 5.0 to 9.0 min (solutions A and B were H₂O and acetonitrile, respectively, both containing 0.5% (v/v) trifluoroacetic acid). The HPLC system used SpetraSYSTEM P2000 gradient pumps, a UV6000LP Diode Array Detector, and the ThermoQuest ChromQuest Chromatography Data System (Thermo Separation Products). The 368-nm chromatograms were used for quantitative analyses. The complete oxidation of either N¹-acetyl-Spm or N¹-acetyl-Spd by PAO was found to produce an equivalent amount of 3-acetamidopropanal. This result further substantiates the conclusion that PAO described herein is the classical mammalian peroxisomal N¹-acetyl-polyamine oxidase (Höltta, Biochemistry 16:91-100 [1977]; Bolkenius and Seiler, Int. J. Biochem. 13:287-292 [1981]; Höltta, Methods Enzymol. 94:306-311 [1983]; Seiler, Prog. Brain Res. 106:333-344 [1995]).

3-Acetamidopropanal and 3-aminopropanal were synthesized using a method familiar to those skilled in the art of organic synthesis. A solution of 1-amino-3,3-diethoxypropane (the diethyl acetal of 3-aminopropanal) (Acros Organics) in dry pyridine was reacted with acetic anhydride to produce 1-acetamido-3,3-diethoxypropane (the diethyl acetal of 3-acetamidopropanal). This compound has not been previously described. After removing most of the pyridine and acetic anhydride by rotary evaporation at high vacuum, the resulting liquid was purified by distilling at high vacuum: bp 111° C. (0.45 mm Hg). 1-Acetamido-3,3-diethoxypropane and 1-amino-3,3-diethoxypropane were hydrolyzed by mixing 1 volume of either with 10 volumes of 1.5 N HCl. Within 1 min, these diethyl acetals were converted to 3-acetamidopropanal and 3-aminopropanal, respectively, which were used immediately for the analysis described in the previous paragraph.

### EXAMPLE 7

### Analysis of mPAO and hPAO Expression

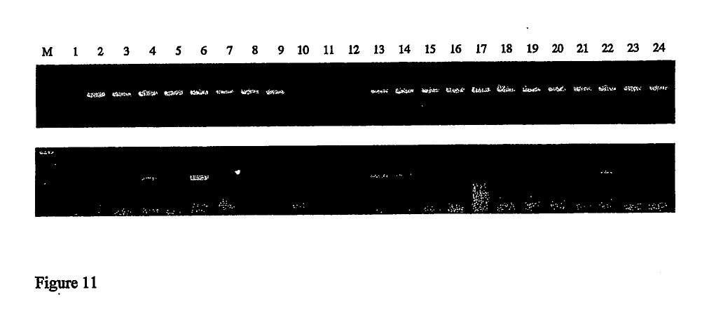

In this Example, methods used to determine the tissue specificity of mPAO and hPAO transcription are described. The “Rapid Scan Gene Expression Panel” (Origin) was used to examine mPAO expression in various mouse tissues and developmental stages. Briefly, total RNA from each sample was subjected to oligo-dT selection, and the first strand cDNAs were generated from poly-A+ mRNA using oligo-dT primers and Moloney murine leukemia virus (MMLV) reverse transcriptase. A 540 bp fragment of mpao was PCR amplified from mouse cDNA samples using two gene specific primers: sense primer, 5′-TCGGAAGAGA ACCAGCTTGT GG-3′ (SEQ ID NO:12); and antisense primer 5′-CAATGACATG ATGTGCAGGC A-3′ (SEQ ID NO:13). As a control, a 570 bp portion of the β-actin gene was also PCR-amplified, using primers provided by the manufacturer. The 24 mouse cDNA samples were serially-diluted over a 4-log range and arrayed into a 96-well PCR plate. The PCR reaction was carried out as follows after a 3 min hot start at 94° C: 35 cycles of 94° C. for 30 sec, 55° C. for 1 min and 72° C. for 2 min. The amplified fragments were electrophoresed on an agarose gel and the fluorescence intensity of the ethidium bromide-stained bands was used to measure relative expression levels of β-actin in the upper panel and murine PAO in the lower panel of FIG. 11. In this figure, M denotes the lane containing the DNA standards, and the numbered lanes represent mRNA from the following tissues: 1) brain; 2) heart; 3) kidney; 4) spleen; 5) thymus; 6) liver; 7) stomach; 8) small intestine; 9) muscle; 10) lung; 11) testis; 12) skin; 13) adrenal gland; 14) ovary; 15) uterus; 16) prostate gland; 17) 8.5 day old embryo; 18) 9.5 day old embryo; 19) 12.5 day old embryo; 20) 19 day old embryo; 21) virgin breast; 22) pregnant breast; 23) lactating breast; and 24) involuting breast. Embryo ages are given in days post-conception. The low levels of β-actin DNA in lanes 1, 10, 11 and 12 are due to a supplier's error. As shown in FIG. 11, the relative mPAO expression levels were found to be: liver>adrenal gland≈ovary≈pregnant breast>spleen≈lactating breast>19 day old embryo>heart≈12.5 day old embryo>uterus>9.5 day old embryo>stomach≈small intestine≈involuting breast>thymus≈muscle≈lung>prostate (barely detectable). The levels of mPAO in lanes 10-12 (e.g., lung, testis, and skin), could not be estimated due to the low levels of mRNA in these samples.

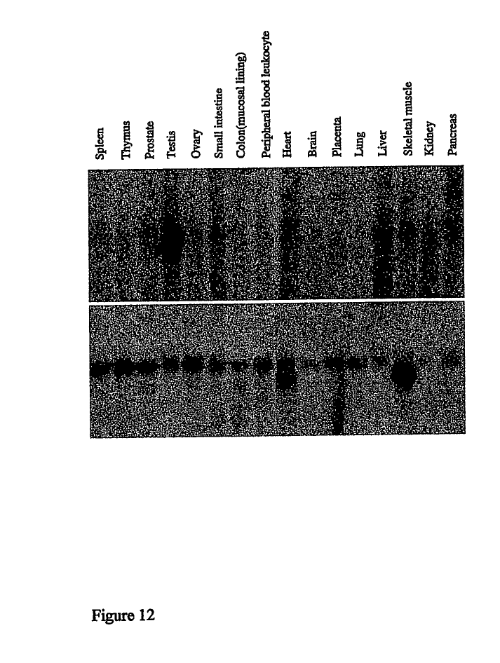

Additionally a multiple tissue northern (MTN) blot was probed with a ³²P-labeled fragment of human PAO to determine the distribution of PAO mRNA in human tissues. The MTN blot (Clontech) contained approximately 2 μg of polyA+ RNA per lane from 16 different human tissues. The hPAO probe was generated from the human EST containing plasmid (GenBank) after Hind III and Eco RI digestions. The hPAO fragment was labeled with ³²P-dATP via a random-primer labeling method. Hybridization was carried out at 65° C. for 3 brs using the ExpressHyb hybridization solution (Clontech) per the manufacturer's instructions. After hybridization the blot membranes were exposed on X-ray film. As shown in FIG. 12, the relative hPAO expression levels were found to be: testis>>>>liver>heart≈skeletal muscle≈pancreas≈kidney>spleen≈prostate>ovary≈small intestine≈peripheral blood leukocytes≈brain>placenta>lung≈colon≈thymus.

### EXAMPLE 8

### Analysis of hpao Expression in Cancer Cells

In this example, the sequencing of hpao and expression of hpao mRNA from human tumor cells is described. By screening GenBank using the hpao sequence as the probe, an altered form of hpao mRNA was found (GenBank Accession No. AW662266) in a sequence derived from a genitourinary tract high-grade transitional cell tumor (TCC). Upon completely sequencing the insert of this cDNA clone, a 10-bp deletion was observed at the splice junction of Exons III and IV (See, FIG. 6, Pane D). This deletion is predicted to result in early termination of hpao mRNA translation and to produce an inactive, truncated form of hPAO. A short repeat (CTTAGG) occurs within a 16-bp fragment 5′-CTTAGGTTTT CTTAGG-3′ (SEQ ID NO:15) in the spliced mRNA of full-length hpao. While the short repeat sequence suggests that improper splicing had occurred to produce the 10-bp deletion 5′-TTTTCTTAGG-3′ (SEQ ID NO:16). It is not known whether this deletion occurred within the genome or whether it occurred during transcription in the TCC cells. Additionally, a search of the human EST bank uncovered an EST clone from a Soares ovary tumor, that lacks V (GenBank Accession No. AA293017), and a colon cancer-derived EST that lacks Exon IV (GenBank Accession No. AW973180). In contrast, hpao cDNA isolated and sequenced from normal human liver, placenta and testis, were found to be complete. It is therefore contemplated that alterations in hpao may contribute to the initiation and/or progression of some cancers.

Interestingly, an hpao sequence derived from a fetal brain tissue clone (GenBank Accession No. BI91922), which contains an insert extending from hpao Exon I to Exon VI, is missing the 3′-end of Exon II and all of Exon IV. Thus, altered transcription of hpao mRNA is contemplated to occur in fetal tissue.

In addition, a reverse-transcriptase-PCR (RT-PCR) experiment was performed to analyze the expression of hpao in OVCAR-3 and HL-60 cells. Briefly, about 10⁶ to 10⁷ cells were harvested and the total RNA of each sample was isolated by using the RNeasy miniprep kit (QIAGEN). A 1 μg mRNA sample was used for cDNA synthesis with the MMLV reverse transcriptase following the manufacturer's instructions provided with the Advantage RT-for-PCR Kit (Clontech). Human liver and placenta polyA mRNA were also obtained from Clontech. cDNA amplification was achieved following the SMART RACE™ cDNA Amplification method (Clontech). A pair of gene specific primers was generated based on the hpao cDNA sequence; hpao1 was the sense primer 5′-AGGCAGCCTT TCCCGGGGAG ACCTTTC-3′ (SEQ ID NO:17), and hpao2R was the antisense primer 5′-TCTCCATGAA CTCAGACTCA AGT-3′ (SEQ ID NO:18). Positive control amplimers for human G3PDH (glyceraldehyde-3-phosphate-dehydrogenase) were also used; the sense primer was 5′-TCCACCACCC TGTTGCTGTA G-3′ (SEQ ID NO:19) and the antisense primer was 5′-GACCACAGTC CATGCCATCA CT-3′ (SEQ ID NO:20). PCR was performed at 94° C. for 3 min, 25 cycles at 94° C. for 10 sec, 68° C. for 1 min, and 72° C. for 1 min.

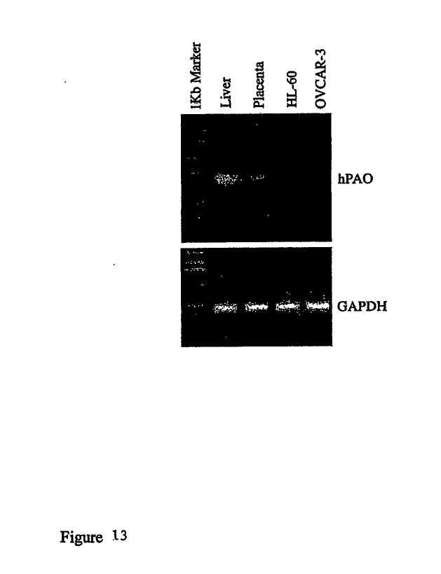

The results of the RT-PCR experiment indicated that the levels of hpao mRNA were very low in both OVCAR-3 and HL-60 carcinoma cells in comparison with that observed in human liver and placenta cells (See, FIG. 13). The decrease in hpao mRNA is contemplated to be due to down regulation of hpao transcription in tumor cells. Moreover, since tumor suppressor genes are frequently down regulated in cancer cells, it is contemplated that hpao functions as a tumor suppressor gene.

### EXAMPLE 9

### Analysis of hpao Expression During Apoptosis

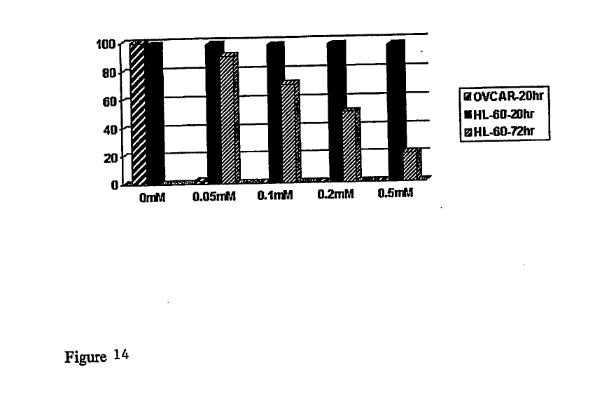

In this Example, the method used to examine hpao mRNA production by cultured cells undergoing apoptosis is described. Briefly, cell death was induced by adding N¹-acetyl-Spm to the growth medium of OVCAR-3 human ovarian carcinoma cells, and HL-60 human promyelogenous leukemia cells that had undergone 48 hrs of growth. Approximately 50 μM N¹-acetyl-Spm killed 100% of the OVCAR-3 cells within 20 hrs. HL-60 cells, in contrast, survived for 24 hrs at all tested N¹-acetyl-Spm concentrations. In addition, although 100% of the HL-60 cells survived a 72 hrs exposure to 0.05 mM N¹-acetyl-Spm, 30%, 50% and 80% of these cells were killed in the presence of 0.1 mM, 0.2 mM, and 0.5 mM N¹-acetyl-Spm, respectively (See, FIG. 14). Clearly both OVCAR-3 and HL-60 cells can be induced to undergo apoptosis by N¹-acetyl-Spm, but with different susceptibilities. The results of a reverse-transcriptase-PCR experiment indicated that significant levels of hpao mRNA were induced in OVCAR-3 cells very quickly after exposure to N¹-acetyl-Spm. There was no change in the level of hpao mRNA levels in the HL-60 cell line during a 48 hr exposure to high concentrations of N¹-acetyl-Spm, which correlated with a decreased susceptibility of these cells to N¹-acetyl-Spm-induced apoptosis.

### EXAMPLE 10

### Structure of mPAO

In this Example, the analysis of the structure of mPAO was initiated with an alignment of several known flavoprotein amine oxidase amino acid sequences. The alignment, which was accomplished using the CLUSTALW (version 1.8) server found at the European Bioinformatics Institute website, was refined further manually. The manual aligning and alignment display was done using the Windows program GeneDoc (version 2.6.002) available at the Pittsburgh Supercomputing Center Biomedical Initiative website. The sequences used to produce this alignment included: peroxisomal bovine PAO (bPAO), peroxisomal human PAO (hPAO), peroxisomal murine PAO (mPAO), cytosolic human Spm oxidase (GenBank Accession No. AY033889; NCBI Accession No. BAA91360; Wang, et al., supra [2001]; Vujcic, et al., supra [2002]), cytosolic murine Spm oxidase (GenBank Accession No. BC004831; NCBI Accession No. AAH0483 1; Vujcic, et al., supra [2002]), human MAO-A (hMAO-A; GenBank Accession No. M69226), human MAO-B (hMAO-B; GenBank Accession No. M69177), Salmo gairdneri (fish) MAO (fMAO; GenBank Accession No. L37878), Mycobacterium tuberculosis amine oxidase (Mt-AmOx; GenBank Accession No. AL021646), Micrococcus rubens putrescine oxidase (Put-Ox; GenBank Accession No. D1251 1), Zea mays (corn) PAO (cPAO; GenBank Accession No. AJ002204), Micro luteus tyramine oxidase (Ml-TyrOx; GenBank Accession No. 3298360), Aspergillus niger MAO (MAO-N; GenBank Accession No. L38858), and Candida boidinii N¹-acetyl-Spd oxidase (CB_N-SpdOx; GenBank Accession No. AB018223). Only the cPAO sequence has a recognizable N-terminal transport signal sequence. In contrast, a tripeptide peroxisomal transport signal is present at the C-termini of bPAO, hPAO, mPAO, MAO-N and Cb_N-SpdOx; peroxisomal transport consensus sequence=-[S/A/C/P]-[K/H/R]-[I/L/M](Gould, et al., J. Cell Biol. 108:1657-1664 [1989]). Extended C-terminal regions of hMAO-A, hMAO-B and fMAO are thought to be anchors that hold these proteins to the outer surface of mitochondria (Binda, et al., Nature Struct. Biol. 9:2-26 [2002]).

From the alignment, 2 highly conserved regions were identified: one near the N-termini of these enzymes is a clearly identifiable β-α-β consensus domain, which interacts with the ADP moiety of FAD (Schilling and Lerch, Biochim. Biophys. Acta 1243:529-537 [1995]; and Wierenga, Terpstra, and Hol, J. Mol. Biol. 187:101-107 [1986]); the other located at the C-termini is also involved in FAD binding. Moreover, located close to the C-termini is a moderately conserved region that harbors the Cys residues of the monoamine oxidases that are covalenily linked to FAD: Cys⁴⁰⁶ (hMAO-A) and Cys³⁹⁷ (hMAO-B). In bPAO and mPAO, a Ser (Ser⁴²⁹ of mPAO) aligns with these Cys residues. The CLUSTALW analysis provided the following percent identities (percent similarities) between mPAO and other flavin-containing amine oxidases: hPAO, 79% (89%); bPAO, 73% (82%); cytosolic human Spm oxidase, 36% (53%); cytosolic murine Spm oxidase, 36% (53%); Micrococcus rubens Put-Ox, 19% (32%); cPAO 19% (34%); Salmo gairdneri MAO, 17% (30%); human MAO-B, 16% (30%); Mycobacterium tuberculosis amine oxidase, 16% (30%); Candida boidinii N¹-acetyl-Spd oxidase, 16% (32%); human MAO-A, 15% (30%); Micro luteus tyramine oxidase, 13% (28%); Aspergillus niger MAO-N, 12% (25%). Overall, the amino acid sequence identity between mPAO and the other flavoprotein amine oxidases is rather low (e.g., generally less than 20%, except for the 36% identity to the newly discovered human and murine Spm oxidases). Thus, it is contemplated that the bovine, murine and human peroxisomal PAOs described herein, represent a new subclass of mammalian amine oxidases.

Even so, there appears to be a great deal of conservation of the basic structural elements of mPAO and cPAO. For example for mPAO, the positive-ends of the α-helical dipoles that interact with the diphosphates (the sequence from residues 14-26) and the N1/C2 O locus of FAD (the sequence from residues 475-491) are conserved, as are some elements of the Rossmann fold (the sequences from residues 6-38 corresponding to the βαβ motif, 261-282, 291-305, 420-431, and 476-494) (Dym and Eisenberg, Protein Sci 10:1712-1728 [2001]). Three conserved regions that are close to and possibly interact with FAD and substrate, are those defined by residues 38-50, 56-64, and 216-243. Two other conserved regions that are remote from FAD and the substrate-binding site are located at residues 315-325 and 371-376. Therefore, the structure of mPAO was modeled using the known cPAO structures (Binda et al., Structure 7:265-276 [1999]) as templates. The coordinates of the 1.9 angstrom X-ray structure for corn PAO (cPAO) and the cPAO/MDL75257 complex are available in PDB files 1B37 and 1B5Q respectively (Binda et al., supra [1999]). Initially the PC program Swiss-PdbViewer version 3.5b3, found on the ExPASy Molecular Biology Server website, displays the three-dimensional structures of cPAO/MDL template and a linear α-helix structure of the target protein mPAO. Using the “Magic Fit” option, the program threads the mPAO sequences onto the cPAO structure. The program displays the superimposed structures of the two oxidases and then the second template cPAO, is superimposed onto these structures. The sequence alignment of all three proteins is shown on the computer monitor. The fit of the mPAO sequence to the cPAO/MDL structure was refined further by using the “Iterative Magic Fit” option. Visual adjustments were made to the sequence alignments, resulting in changes to the predicted crude mPAO structure (e.g., gaps in the crude mPAO structure are spanned by unusually long bonds). Multiple “good” crude mPAO structures were generated in this way, each having a different alignment with the templates. The coordinates of each crude mPAO structure and the corresponding structural information for the templates, cPAO/MDL and cPAO, in PDB format, were sent to SWISS-MODEL located at the ExPASy Molecular Biology Server web site. With this site, molecular mechanic energy minimizations of the crude mPAO structures were carried out (Optimized Mode) using the GROMOS96 force field program. The program failed to provide valid structures for about 30-40% of the crude models (i.e., GROMOS96 program crashed). For the remaining models, PDB coordinate files with “refined” mPAO structures were obtained. Each file also contained the structures for cPAO and cPAO/MDL. When any file was viewed with the Swiss-PDB Viewer, all three structures were superimposed. Those portions of the modeled mPAO structure that were ill-defined or that produced unfavorable interactions were displayed in red. The inspection of twelve mPAO structures, indicated that, while the overall tertiary structures differed, the stereochemistry of the substrate and FAD binding sites were remarkably well conserved. One structure with the fewest ill-defined regions and unfavorable interactions was selected. The PDB file for this structure was edited with a word processor to remove all the coordinates for the cPAO and the cPAO/MDL structures except for the coordinates for the FAD of cPAO, yielding a PDB file with the coordinates for the “refined” mPAO structure bound to FAD.

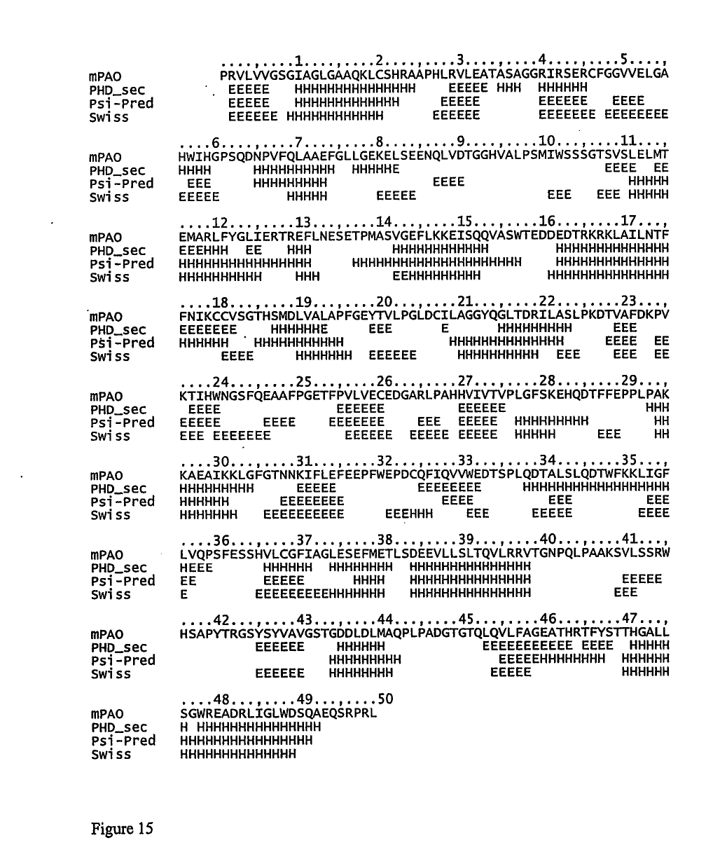

The mPAO amino acid sequence was sent to the “Predict Protein” web site of the European Molecular Biology Laboratory, and the “Psi-Pred” web site of the University College London, which returned predicted secondary structures for mPAO. These secondary structures were compared with that for the Swiss Model 3-D structure of mPAO/MDL. As shown in FIG. 15, the secondary structure for Psi-Pred shows a secondary structure very similar to that derived from the modeled mPAO structure.

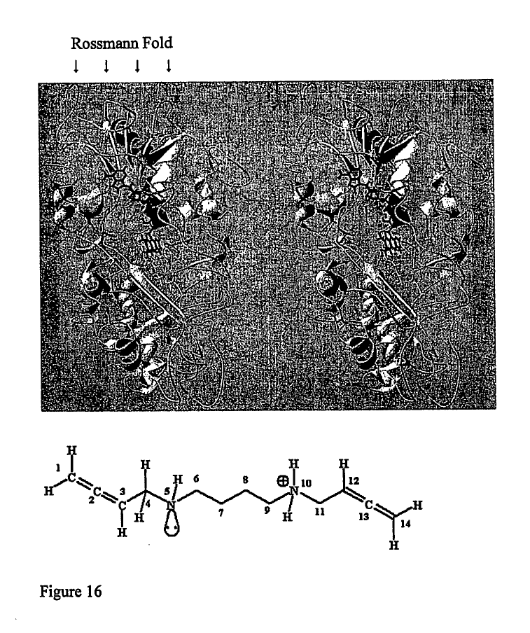

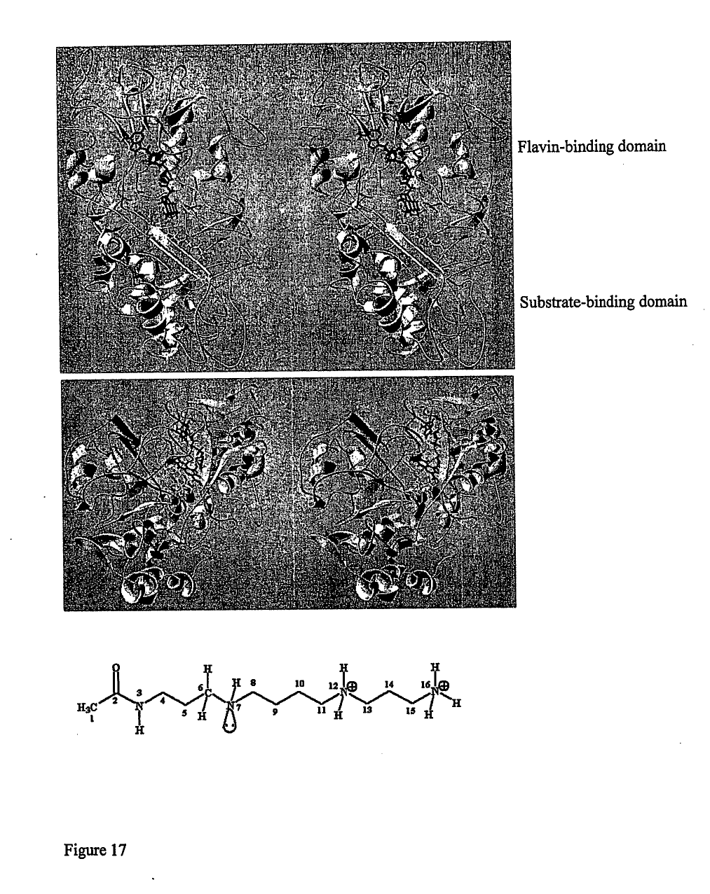

The mPAO structure was refined further by performing a molecular mechanics energy minimization using the CHARMM22 program running on a DEC Alpha computer, resulting in some minor changes. Next the MDL 27527 structure from cPAO was placed into the original GROMOS96 mPAO structure, in order to CHARMM-minimize the energy of the structure. The substrate, N¹-acetyl-Spm was placed into the active site of mPAO and the energy of this complex was again minimized with CHARMM. The ribbon structures of the mPAO/MDL 72527 complex is shown in FIG. 16, and the ribbon structures of the mPAO/N¹-acetyl-Spm complex is shown in FIG. 17. A large number of acidic amino acyl residues are in the vicinity of the surface entrance to substrate binding channel : Glu⁵², Glu⁸⁴, GlU⁸⁵, Asp⁹⁰, Asp²⁰⁶, Asp²⁸⁵, Glu³¹⁴, Glu²¹⁶, Glu³¹⁷, Glu³²¹, Asp³²³, Glu³³², Asp³³³, Asp³³⁹, Glu³⁶², and GlU³⁸⁰.

As with the cPAO x-ray structure (Binda, et al., supra [1999]), the mPAO modeled structure is composed of two distinct domains, a substrate-binding domain and a flavin-binding domain. The substrate-binding domain is composed of the following segments of the modeled mPAO structure: Ser88-Cys¹⁸⁵ and Gly³⁰⁹-Arg⁴¹⁹. The flavin-binding domain consists of mPAO segments: Met¹-Leu⁸⁷, Cys¹⁸⁶-Leu³⁰⁸ and Trp⁴²⁰-Leu⁴⁹⁹ (FIG. 5 and FIG. 17). At the interface of these domains, there are numerous amino acyl residues that are involved in substrate and inhibitor binding (supra). Much of the FAD-binding domain of mPAO is defined by a classical Rossmann fold (Dym and Eisenberg, supra [2001]), which interacts primarily with ribityl-ADP moiety of the flavin (top part of the protein structure in FIG. 16) (supra).

A continuation of these modeling studies is contemplated. In particular, the anticonvulsant, milacemide is placed in the active site of mPAO and the energy of this complex is then minimized with CHARMM force field parameters. Similarly, the substrate MDL 27659 is placed in the active site of mPAO for energy minimization. Further refinements are carried out by energy minimizing molecular mechanics calculations of the mPAO and mPAO/X complex structures in a water box. Now that the structure of MAO-B is known (Binda et al., Nature Struct Biol 9:22-26 [2002]), it will be possible to use simultaneously the structures of cPAO and MAO-B as templates to model the structures of mPAO and hPAO.

Since the structures of the X component (e.g., N¹-acetyl-Spm, MDL 72527, milacemide, and MDL 27695) of the complex vary considerably, these studies provide an idea of the flexibility, size, and topology of the active site of the PAO/X complexes. The stereochemistry in the vicinity of the active site of these complexes also indicates those groups that are essential for binding and oxidation of any of these secondary amine substrates. This information is invaluable for designing drugs that are more or less specific to PAO. As the substrate-binding pocket in the vicinity of FAD is quite large, the addition of a side group to the substrate or inhibitor is contemplated to increase or decrease specificity as needed.

The active site structures of modeled mPAO and the X-ray structure of cPAO have been compared. All residues within a 15 angstrom sphere from the N5-position of FAD of the superimposed isoalloxazine rings were viewed. There are significant differences, as well as similarities. This finding is not unexpected given that the natural substrates for mPAO and cPAO are different. The substrates for mPAO are N¹-acetyl-Spm and N¹-acetyl-Spd. In contrast, the substrates for cPAO are Spm and Spd, which are poor substrates for mPAO. Additionally, the carbon centers oxidized by mPAO and cPAO differ.

The veracity of the “best” mPAO structure was tested by sending its PDB coordinate to the PROCHECK web site of the University College London (Laskowski et al., J. Appl. Cryst. 26:283-291 [1993]). For the PROCHECK analysis the “resolution” was automatically set to 2.0 angstroms. The PROCHECK program “checks the bond lengths, bond angles, peptide and side-chain ring planarities, chirality, main-chain and side-chain torsion angles, and clashes between nonbonding pairs of atoms” (Marti-Renom et al., Ann. Rev. Biophys. Biomol. Struct. 29:283-291 [2000]). The results of this analysis indicated that the structure had very few bad or unusual structural features. For example, a Ramachandran analysis indicated that 83.5% of the residues are in the most favored region, 13.7% are in the allowed regions, 1.4% in generously allowed regions and 1.4% in the disallowed regions. Also the structure did not show any aromatic ring or peptide bond distortions and there were only a few residues with “distorted” bond lengths or bond angles.

In summary, the basic features of the stereochemistry of the active site are well-represented by the structures determined in this Example. This conclusion is based upon the finding that when the substrate is randomly-positioned within its binding site in mPAO, the carbon atoms from which a hydrogen is removed aligned optimally with the N5-position of the flavin isoalloxazine ring. Apparently, the residues Glu⁸⁴ and ASP³³⁹ are crucial for providing a binding register by interacting with the amino groups of the substrate.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in relevant fields, are intended to be within the scope of the present invention.

1. An isolated nucleic acid that comprises a sequence selected from the group consisting of a gene encoding a peroxisomal acetylpolyamine oxidase protein of a mammal, a gene encoding a biologically active portion of said peroxisomal acetylpolyamine oxidase protein, and a gene encoding a biologically active variant of said peroxisomal acetylpolyamine oxidase protein.

2. The isolated nucleic acid of claim 1, wherein said mammal is selected from the group consisting of a cow, a mouse and a human.

3. The isolated nucleic acid of claim 2, wherein said nucleic acid is selected from the group consisting of the open reading frames of SEQ ID NO:1, SEQ ID NO:3, and SEQ ID No:5.

4. The isolated nucleic acid of claim 2, wherein said nucleic acid encodes a protein selected from the group consisting SEQ ID NO:2, SEQ ID NO:4, and SEQ ID NO:6.

5. The isolated nucleic acid of claim 1, wherein said nucleic acid encodes a protein with amine oxidizing activity.

6. The isolated nucleic acid of claim 5, wherein said substrate for said amine oxidizing activity is selected from the group consisting of N¹-acetyl-Spm, N¹-acetyl-Spd, N¹, N¹²-diethyl-Spm, N¹, N¹¹-diethyl-nor-Spm, and Spm.

7. A vector comprising the nucleic acid of claim 1.

8. The vector of claim 7, further comprising a promoter operatively linked to said nucleic acid.

9. A host cell comprising the vector of claim 7.

10. The host cell of claim 9, wherein said host cell is located in an animal.

11. A host cell comprising a disruption of the gene of claim 1.

12. The host cell of claim 11, wherein said host cell is located in an animal.

13. An isolated mammalian nucleic acid sequence selected from the group consisting of the open reading frames of SEQ ID NO:1, SEQ ID NO:3, SEQ ID NO:5 and sequences that hybridize to the complement of the open reading frames of SEQ ID NO:1, SEQ ID NO:3, SEQ ID NO:5, under conditions of low stringency, and wherein said isolated nucleic acid sequence encodes a polypeptide having amine oxidizing activity.

14. A composition comprising an isolated protein selected from the group consisting of a peroxisomal acetylpolyamine oxidase of a mammal, a biologically active portion thereof, or a biologically active variant thereof.

15. The composition of claim 14, wherein said mammal is selected from the group consisting of a cow, a mouse and a human.

16. The composition of claim 15, wherein said peroxisomal acetylpolyamine oxidase comprises a sequence selected from the group consisting of SEQ ID NO:2, SEQ ID NO:4, and SEQ ID NO:6.

17. The composition of claim 14, wherein said peroxisomal acetylpolyamine oxidase has amine oxidizing activity.

18. The composition of claim 17, wherein the substrate for said amine oxidizing activity is selected from the group consisting of N¹-acetyl-Spm, N¹-acetyl-Spd, N¹, N¹²-diethyl-Spm, N¹,N¹¹-diethyl-nor-Spm, and Spm.

19. The composition of claim 14, wherein said peroxisomal acetylpolyamine oxidase is a recombinant peroxisomal acetylpolyamine oxidase protein.

20. The composition of claim 19, wherein said peroxisomal acetylpolyamine oxidase protein further comprises an affinity tag.

21. A method for detecting mammalian peroxisomal acetylpolyamine oxidase expression in a cell comprising the steps of:
a) providing:
i) a sample from a mammalian subject, and
ii) at least one reagent capable of specifically detecting mammalian peroxisomal acetylpolyamine oxidase expression; and
b) contacting said sample with said at least one reagent under conditions suitable for binding said at least one reagent to a mammalian peroxisomal acetylpolyamine oxidase gene product.

22. The method of claim 21, wherein said mammalian peroxisomal acetylpolyamine oxidase gene product comprises mRNA, and wherein said at least one reagent comprises a nucleic acid probe of at least 12 nucleotides in length that specifically hybridizes under conditions of high stringency to said mRNA or to cDNA corresponding to said mRNA.

23. The method of claim 22, wherein said contacting is accomplished by a technique selected from the group consisting of polymerase chain reaction and Northern blotting.

24. The method of claim 21, wherein said mammalian peroxisomal acetylpolyamine oxidase gene product comprises protein, and wherein said at least one reagent comprises an antibody that binds to said protein.

25. The method of claim 24, wherein said contacting is accomplished by a technique selected from the group consisting of enzyme-linked immunosorbent assay, Western blotting, immunofluorescence analysis, immunohistochemistry and flow cytometry.

26. The method of claim 25, wherein said antibody further comprises a reporter molecule selected from the group consisting of an enzyme and a fluorochrome.

27. A method of inhibiting mammalian peroxisomal acetylpolyamine oxidase activity comprising:
a) providing a mammalian peroxisomal acetylpolyamine oxidase, and an inhibitor; and
b) contacting said mammalian peroxisomal acetylpolyamine oxidase with said inhibitor under conditions suitable for reducing amine oxidizing activity of said oxidase.

28. The method of claim 27, wherein said inhibitor is selected from the group consisting of synthalin and N-(3-aminopropyl)-1,10 decanediamine.

29. The method of claim 27, wherein said mammalian peroxisomal acetylpolyamine oxidase is located in a cell or in an animal.

30. A method comprising:
a) providing a host cell comprising an exogenous nucleic sequence selected from the group consisting of the open reading frames of SEQ ID NO:1, SEQ NO:3, SEQ D NO:5 and sequences that hybridize to the complement of the open reading frames of SEQ ID NO:1, SEQ ID NO:3, SEQ ID NO:5 under conditions of low stringency, wherein said isolated nucleic acid sequence encodes a polypeptide having amine oxidizing activity; and
b) culturing said host cell under conditions such that said exogenous nucleic acid sequence is expressed.


SEQUENCE LISTING (23 sequences)

SEQ ID NO: 1 - DNA, 1625 bp, Bos taurus
ggtggcgtggtggaggtaggtgctcactggatccatgggccctcccagggcaaccccgtcttccagctggctgccaaatacgggctgcttggggagaaggccctgtccgaggagaaccagctgatcgagaccggaggtcacgtgggcctgccatctgtgtcttacgccagctctggggtaagtgtgagccttgagctcgtggcggagatggccagtctgttctacagtctcatagaccagaccagggagttcctgcaggcggctgagaccaccccacccagtgtcggggagtacctcaaggagaagatccgtcagcacatggctggctggacagaggatgaggagaccaagaagctcaaactggccatcctgaagaacttgttcaacgtggagtgctgtgtgagcggtacccacagcatggacctggttgccctcgcgcctttcggggagtacactgtgctgccagggctggactgcacctttcctgagggctaccaaggactcacagactgcatcatggcctccttgcccaaggacgtgatggtttttgacaaacccgtgaagaccattcactggaatgggtccttccgggaagcttctgctcctggggagacatttcccgtgctggtggaatgtgaggatggagactgcttcccagcccatcacgtggtcgtcaccgtgcccttaggtttttttaaaaagcatctggacaccttctttgagccaccactgcccactgaaaaggtggaagcgatcaggaaaataggctttgggacgaacaataaaatcttcttggagtttgaggagcccttctgggagccagactgccagcacatccaggtggtgtgggaggacatgtcgcccctggaggataccgcccccgagctgcaggatgcctggttcaagaagctgattggcttctgggtcctgcctcccttccaggccagccacgtgctctgtggctttatcgcggggctcgagtccgagttcatggagacactgtcggacgaggacgtgctcaggtctctgacgcaggtgctccgcagggtgacagggaacccccagctccccgcacccaggagtatgctgaggtcctgctggcacagcgccccgtacacccggggctcatacagctacgtggccgtgggcagctctggggacgacatggaccggctggcccagcccctcccttcagacggcaagggggcacagctccaggtactgttcgcaggggaagccacacaccggacgttttattcccccacacacggggctctgctgtctggctggagggaggctgaccggctcatgacgctgtgggacccccaggcccagtggcccgagcccaggctctgagcctggctctgcgctcccagtgctagggttgaggaggctggccctatcctgtcctgtgatggatgaggttcaggtgggcttgggatgttgtcaccgggggtcagctccctcatgtctcaggttgctggattccggtcagggctgagcttgagctcagtgcagtcttcctggaggacggagacatgggatgctgctgcccccgccattgatggcacataaagtgtgtaaaacagtcaaaaaaaaaaaaaaaaaaaaaaaaaaaaaaaaaa

SEQ ID NO: 2 - protein, 456 aa, Bos taurus
SEHSFGGVVEVGAHWIHGPSQGNPVFQLAAKYGLLGEKALSEENQLIETGGHVGLPSVSYASSGVSVSLELVAEMASLFYSLIDQTREFLQAAETTPPSVGEYLKEKIRQHMAGWTEDEETKKLKLAILKNLFNVECCVSGTHSMDLVALAPFGEYTVLPGLDCTFPEGYQGLTDCIMASLPKDVMVFDKPVKTIHWNGSFREASAPGETFPVLVECEDGDCFPAHHVVVTVPLGFFKKHLDTFFEPPLPTEKVEAIRKIGFGTNNKIFLEFEEPFWEPDCQHIQVVWEDMSPLEDTAPELQDAWFKKLIGFWVLPPFQASHVLCGFIAGLESEFMETLSDEDVLRSLTQVLRRVTGNPQLPAPRSMLRSCWHSAPYTRGSYSYVAVGSSGDDMDRLAQPLPSDGKGAQLQVLFAGEATHRTFYSPTHGALLSGWREADRLMTLWDPQAQWPEPRL

SEQ ID NO: 3 - DNA, 1770 bp, Mus musculus
agacaaagactgttctaaaggcggcctaaggccaggtgtctggaggaccctgagctagccatggcgttccctggcccgcgggtgctggtggtgggcagcggcatcgcggggctgggcgctgcgcagaagctctgcagccaccgggctgctccacacctgcgggttctggaggccaccgccagcgccgggggccgcatccgctcggaacgctgcttcggtggtgtagtggagctgggtgcacactggatccatgggccctcccaggacaatcctgtcttccagctggctgcagaattcgggctcctgggggagaaagaattatcggaagagaaccagcttgtggatacaggagggcacgtggccctgccctccatgatctggtccagctctggaacaagtgtgagcctggagctgatgacagagatggcccgtctgttctatggactgatagagcggacccgagaattcctgaatgaatctgaaaccccaatggccagtgttggagagttcctgaagaaggagataagtcagcaggtggctagctggaccgaggatgatgaggacaccagaaagcgcaagctggctatcctgaatactttcttcaacatagagtgttgtgtgagcggcacccacagcatggacctggtggctcttgcaccgttcggggagtatacagtgctgcctgggctggattgcatcttggctggtggctaccaaggacttaccgaccgcatactggcttccttgcccaaggatacggtcgcttttgacaagccggtgaagaccattcactggaatgggtccttccaggaagctgcttttccaggggagaccttccccgtattggtggagtgtgaagatggtgcccgcctgcctgcacatcatgtcattgtcacagtgccattaggttttcttaaagaacaccaagataccttttttgagccaccactgcctgccaaaaaagcagaagccatcaaaaaactaggctttggtaccaacaacaaaatcttcttggagtttgaagagcccttctgggagcccgactgccagttcatccaggtggtgtgggaagatacatcgcccttacaggacacagccctctcactccaagacacctggttcaagaagctcattggatttttggtccagccttcttttgagtcttcacatgtgctctgtgggtttattgctgggctggagtcggagtttatggagactctgtcagatgaagaagtgcttctgtctctaacccaagtactccggagggtgacaggaaacccacaattgccagcagccaagagcgtgctgagttctcgatggcacagtgccccatacacccgagggtcctacagctatgtggctgtgggcagcactggcgatgacctagatctgatggctcagcctctcccagcagatggaactggcacccagctccaggtactctttgctggggaagccacacatcggacattttattctaccacacacggagccctcctgtctggctggagggaagctgatcgcctcattggtctgtgggactcacaggcggagcagtcccggcccaggctgtgagtgcctgctgctctgctctaccactcgggctgaccaagctctcagtctggtttgaggtttggggatcttactggtagctttaattttgggaaagtggccttgtttcacatatctgttggagtctggcttggtctgaccattgatgccctgctaactgttgtacacataaagcaattaacactgaaaaaaaaaaaa

SEQ ID NO: 4 - protein, 504 aa, Mus musculus
MAFPGPRVLVVGSGIAGLGAAQKLCSHRAAPHLRVLEATASAGGRIRSERCFGGVVELGAHWIHGPSQDNPVFQLAAEFGLLGEKELSEENQLVDTGGHVALPSMIWSSSGTSVSLELMTEMARLFYGLIERTREFLNESETPMASVGEFLKKEISQQVASWTEDDEDTRKRKLAILNTFFNIKCCVSGTHSMDLVALAPFGEYTVLPGLDCILAGGYQGLTDRILASLPKDTVAFDKPVKTIHWNGSFQEAAFPGETFPVLVECEDGARLPAHHVIVTVPLGFSKEHQDTFFEPPLPAKKAEAIKKLGFGTNNKIFLEFEEPFWEPDCQFIQVVWEDTSPLQDTALSLQDTWFKKLIGFLVQPSFESSHVLCGFIAGLESEFMETLSDEEVLLSLTQVLRRVTGNPQLPAAKSVLSSRWHSAPYTRGSYSYVAVGSTGDDLDLMAQPLPADGTGTQLQVLFAGEATHRTFYSTTHGALLSGWREADRLIGLWDSQAEQSRPRL

SEQ ID NO: 5 - DNA, 1874 bp, Homo sapiens
agcggccgctcgcgatctagaactagtccagacctcccggctactcagaagccctcggactgcccggaccgcgcgatggagtcgaccggcagcgtcggggaggccccgggcggaccccgggtgctggtggtgggcggcggcatcgcggggctgggcgcggcgcagaggctctgcggccactccgccttcccgcacctgcgggtcctggaggccacggcccgcgccgggggccgcatccgctcggagcgctgcttcggtggcgtggtggaggtgggcgcgcactggatccatgggccctcccggggtaaccccgtcttccagctggctgctgagtacgggctgctgggggagaaggagctgtcccaggagaaccagctggtggagaccgggggtcacgtgggcctgccctccgtgagctacgccagctccggggccagcgtgagcctccagctggtggcggagatggcgactctgttctacggcctgatagaccagacccgggagttcctgcacgctgcagagaccccggtgcccagcgtcggggagtacctcaagaaggagattggccagcacgtggccggctggacagaggatgaggagaccaggaagctgaagctggccgtcctgaactccttcttcaacctggaatgctgtgtgagcggcacccacagcatggacctggtggccctggcaccctttggggagtataccgtgctgccggggctggactgcaccttttctaagggctatcaaggactcacaaactgcatgatggccgccctgccggaggacactgtagtttttgagaagcctgtgaagaccatccactggaacgggtccttccaggaggcagcctttcccggggagacctttccagtgtcggtagagtgtgaggatggagaccggttcccggcgcaccatgtcatcgtcaccgtgcccttaggttttcttagggaacatttggacaccttctttgaccctcccctgccggctgagaaggcagaagcaatcaggaagataggctttgggaccaacaacaaaatcttcctggagtttgaggagcccttctgggagccagactgccagctgatccagctggtgtgggaggacacgtcgcccctggaggatgctgcccctgagctacaggacgcctggttccggaagctcattggctttgtggtcctgcctgcctttgcgtctgtccacgttctctgtgggttcattgccggacttgagtctgagttcatggagactctgtcggatgaagaagtacttctgtgtctcacccaagtgctccggagagtgacaggaaacccacggctccccgcgcccaagagcgtcctgcggtctcgctggcacagcgccccgtacactagggggtcctacagctacgtggccgtgggcagtactgggggcgacctggacctgctggctcagcccctccctgcagacggcgccggcgcccagctccagatcctgtttgcgggggaagccacacatcgcacgttttactccacgacgcacggggctctgctgtcgggatggagggaggccgaccgcctcctcagtctgtgggccccgcaggtgcagcagcccaggccgaggctctagctgggcccagcctactctgttccacccgtgtcgggggtaggctgggaccgtcatttcttctgacagatttcagtctggcttgaaatttggggatgttaatgagggtcctctggtttttggtaaccagggccaccttctcagtcttgtgtctgttattggagtctggccagggttgacttgagctgagacaccagatgctcacggagatgctggacacataaagcaagttacagccacaaaaaaaaaaaaaaaaaaaaaaaaaa

SEQ ID NO: 6 - protein, 511 aa, Homo sapiens
MESTGSVGEAPGGPRVLVVGGGIAGLGAAQRLCGHSAFPHLRVLEATARAGGRIRSERCFGGVVEVGAHWIHGPSRGNPVFQLAAEYGLLGEKELSQENQLVETGGHVGLPSVSYASSGASVSLQLVAEMATLFYGLIDQTREFLHAAETPVPSVGEYLKKEIGQHAVGWTEDEETRKLKLAVLNSFFNLECCVSGTHSMDLVALAPFGEYTVLPGLDCTFSKGYQGLTNCMMAALPEDTVVFEKPVKTIHWNGSFQEAAFPGETFPVSVECEDGDRFPAHHVIVTVPLGFLREHLDTFFDPPLPAEKAEAIRKIGFGTNNKIFLEFEEPFWEPDCQLIQLVWEDTSPLEDAAPELQDAWFRKLIGFVVLPAFASVHVLCGFIAGLESEFMETLSDEEVLLCLTQVLRRVTGNPRLPAPKSVLRSRWHSAPYTRGSYSYVAVGSTGGDLDLLAQPLPADGAGAQLQILFAGEATHRTFYSTTHGALLSGWREADRLLSLWAPQVQQPRPRL

SEQ ID NO: 7 - protein, 21 aa, Bos taurus
EAEAPGRGPRVLVVGGGIAGL

SEQ ID NO: 8 - protein, 20 aa, Bos taurus
SEHSFGGVVEVGAHWIHGPS

SEQ ID NO: 9 - protein, 16 aa, Bos taurus
LMTLWDPQAQWPEPRL

SEQ ID NO: 10 - DNA, 25 bp, Artificial Sequence - Synthetic
  Synthetic
gttctcttccgataattctttctcc

SEQ ID NO: 11 - DNA, 35 bp, Artificial Sequence - Synthetic
  Synthetic
gcgagctcatacatatggcgttccctggcccgcgg

SEQ ID NO: 12 - DNA, 22 bp, Artificial Sequence - Synthetic
  Synthetic
tcggaagagaaccagcttgtgg

SEQ ID NO: 13 - DNA, 21 bp, Artificial Sequence - Synthetic
  Synthetic
caatgacatgatgtgcaggca

SEQ ID NO: 14 - DNA, 16510 bp, Homo sapiens
aaaaagttctaaactggatgatggtgacagtggcacaactctgtaactaaactgaacaccattgaattgaacactttaaacgggagaagtcttacagcatgatgtctatatcaataaagctattttaaaaaatgaacaagcctgattcagtcaaagccacatgggtagttgccaccttgatcaccataggtggcatcacccaaggccctctggagctaggagaagcacataccttgtaccagtcagatggaaaagaggttgtgtccgtgtgttccagctggtggctctggcatggcaggattgtccaggactggccgtggtcacgtgcactctccatttcagagcaagggggacgagcgactaacccagccccaggtcagcgcgtcagcggcccgtgctattggattcaggctgtggcaccacatccaggggacaaagagcgcgtcggttgtccccaccctgcagctcttgggtccatgtcttctctctccgcgccataaagccagagttcccatttcagtcactacaatcacccctccttgcaccaggccttccaggtgtggggcatgggcatgtttacctctggcctgtgtggaccgctgtggggaggcttggccagcaagttgctgagccaagtatcttttatgggatcctgccgctccacaggaggatccgggaggctgaactagagttaagttagaatctttcggttccccttcgggctgggctccccgcaggaaccaggaacgggctggccaggaggacatgatggaccgggagaaggtgacagaggcaaagccagacccttcctgttccatcttgcagaggctgtcccaacgcgaggtgaggccaggtgcctgggggacatcgatgcctgaccagagcccagcctggacagccttccagcaaaggctgggcactggccgaccgtaacggcccaggagaatgaggacacgggagtctcggggctggaggagctggctgaccagatggtgcctgacacggtgctgaaaacagggcagcagtccccagaggctgaggggccaaagtagcctgtccacccagagtggcgaggcccacgcctgtgcaggaggcctgaggcatggggtggcctccctcacagctgtgcccacccagcctgtgcgcagggcgatgtgctcctatgtctgggcagtgagctgggccctgcggcgagagcagcagcagctcgatttcacacggagccactagggagtgggccgttcccttggccatacccgtgggggacccagttcatctgtggaattcagaaccccagagtatttaatctgccatctcaggacctccgctggggagctggggcaggtgggtgggtggtcccgctgggcttccgtggtgcatgccagggacctcagaatggagcgccccactgagccagcggctctgcttcaggcccaagagcagggagtaagagagaagcctgctcaggcagaacaggtgacactcgtggcgggggctgcaccccaaacacttgccatccgactaagggtgtggggcagcccttctttccttagtcctggcatgtcgggtgggtcagcttggtggacagctggccctgctgacccgggatccgccctgggttccccatggcctggaggcaggaggagatggccactgcatccagcgtcgcctcctggtgctgcgggcccctgacggacagaggacgctcgacccctgcccgccaccgcccagggagtcagcccgactcccaggcacccgcccctctccctgcctggagctcagctcgcggggttggctagggagtgatggggagtggaatccagcgacaccaggtcacccttccgggagccttttctccagctgggacgagagggaatcaaaggaaagtaagacacggctcaggaggcccacgccgagagcgaagagtgaagctcacgctggcaaactggagcgcgcggcccctcggcagaggaccggggctcaggaggcggggacgcgcctgaaaacagggcgggagcctttccaacccccgcgggctgcgatgcagcagaaaactcggccgcctgcaccggggattctccagaaacacaggcgtgcctgggtgtgcgctcaacttcccgggcgagggacgtttctgcacttgccctcggcttctgaactttgtactttgcgagtgtactttgtactttggtcctgtgtccctgcagccctgtgactacacgcgcacacgcaggcgcgcgcacacgcgaactgacggctcgctggcgatgctggaggctttcacagcccccctccggacgcgttaagctctcccaggtcccacccctgacaccgtggcacccccagacccactgcgccctcgcactcgacgcgggtccctgggggaagcgctcgctgcgccctcgtctcccttggaggagacactggcccgcccggccggccggcttctccctccatggggacgctcgcgcgcaccgcccggcagcgcgggacgctccctgacgcctgtcctcgcgggccctcccacgcggggcgcttccctctgagacccgagcccagtccggaggctcgcggaggccgaggcgcactgtggggctcggcccggccccttccccgcccctccccgcccctccccgcccctccccgcgcctccccgcgcctccccgcgcctccccgcgcctccccgcccctccccgcgcctccccgcccctccccgcgcctccccgcccctccccgcccctccccgcccctccccgcgcctccccgcgcctccccgcccctccccgcccctcccagcgcctccccgccccctcgctccgcctccggcctcctccgagagctccagacctcccggctactcagaagccctcggactgcccggaccgcgcgatggagtcgaccggcagcgtcggggaggccccgggcggaccccgggtgctggtggtgggcggcggcatcgcggggctgggcgcggcgcagaggctctgcggccactccgccttcccgcacctgcgggtcctggaggccacggcccgcgccgggggccgcatccgctcggagcgctgcttcggtaaccgcccctcccggagcccctcccggaacccaaccggctgcgcccgaactcgccggccccaacctgccctgcgcgcagccgacctgcccggccgcctcaccgggctccccgagggaatccccgctcgcttaatccgccagagttcaccgccccgaaactcagggctcagagttagtcaggagggcgccggccagggaaggcgacctacggctccacaaggccgccctgattctgccccacgggggcccccccagccccggggtacgcccaccctgcgcccctccctctggtccacaccgcccgacaagtgccctcctgcccaggtgctccccttaaactgggtctgacagggccctgccctgggggaccacagggcccttggggacagtgtggtacatcctccttgggaaggccagcgtgggcgtggcccaggagaagggctcggggtgacccttctaggaaagggacctccaggtggcatctgctgtcccctcgcaggtggcgtggtggaggtgggcgcgcactggatccatgggccctcccggggtaaccccgtcttccagctggctgctgagtacgggctgctgggggagaaggagctgtcccaggagaaccagctggtggagaccgggggtcacgtgggcctgccctccgtgagctacgccagctccggggccagcgtgagcctccagctggtggcggagatggcgactctgttctacggcctgatagaccagacccgggagttcctgcacgctgcagagaccccggtgcccagcgtcggggagtacctcaagaaggagattggccagcacgtggccggctggacagaggatgaggagaccaggaagctgaagctggccgtcctgaactccttcttcaacctggaatgctgtgtgagcggcacccacagcatggacctggtggccctggcaccctttggggagtataccgtgctgccggggctggactgcaccttttctaagtgcgtgcctgagcccctgccccgccagtcctcccaccaggctccccagggcaccggggctggctgagctccgctcttgcttgggtatgggagggacaggagaccatttgtcctccccattccgacagactggttgctcctttccctagttttgctcatttaagccaaaaaagttatttctgagagagagtgtttaaaagaaggaaaggtaaggctgagtgcggtggctcacgcctgtaatcccagcactttgggaggctgaggcaggtggatcatgaggtcaggagttcaagaacagcctggccaacatggtgaaatcccatctctactaaaaatacaaaaattagccgggcgtgatggtgcatgcctgtaatcccagctactcgggaggctgaggcaggagaattgcttgaacctgggaggcggaggttgcagtgagccaagactgcgccattgcactccaacctgggcgacagagtgagactccatctcaaaagagaaaaaaagaagaagagtaaagacttctcttgctcctgtatttatgaggggcttcaactgcagagggggccagacaggccctctccctgtggcagggtggctgccaggtaccaggtgcagatgagaccctcctcgggctgtcccgtcacccctggtgttggccccaaggctcctggccaggcaatgggcatttgtgagtcaggaactctgtggtggtaaggaggaagccctggttccggcagattgccttgagtgctctgtgcccactgaaccctcccttacagcctggagggcaggtatgagcatccttgaggtggaaaaccacagagtggagctgacctccttgctggccctgagcgatggagagctccccgcagctacctttgatgcgggtgggaactcagagcacgtatttccaccagtcctgcccttccagggactgggcctctacgaaaccagcacttccgtctttctaggggctatcaaggactcacaaactgcatgatggccgccctgccggaggacactgtagtttttgagaagcctgtgaagaccatccactggaacgggtccttccaggaggcagcctttcccggggagacctttccagtgtcggtagagtgtgaggatggagaccggttcccggcgcaccatgtcatcgtcaccgtgcccttaggtaggtcaggttttcagcccaaacccccatcccaagtgcccccgcctctgcccttaccctgcctcagttggctctggggcgtttgcttgtgtacgaagctcacattttcgttctagtaagttccagataggtggaaggaatgattctgttatgttttgtagcaattacatgctttggaattaagggtttcaccgtaagaaaaaacgcgagtaagtatgcgtcctgctcgcttggaggtaaccgtttagggttgcgacggccttggagattaaccacaccgcactttgctgcagacaggaaaaggcatgaaaggcctggtctgaaggtcaggcctggggtgacctgttgaaggagccgtgtgtataattctgtttaattctagatctggtgctttagatctgtccttgagtagaggtggtttgaagctgttttataaaaggtggagatggttgaaaacagagttactaggataattgctgtcaactggacatttgcttcccacgggtttcacgctgccgtgggtctctgcctgagttatctcattgactcaggcagccttgggaggtgtctgtcattgtcccacatatgacagatttgaacatctaggcaagtagagagaagtcacttaattgcccaagttcacaaagctagcgattgccagcactcacttggaaatctaggtgtgattgacaggtgtgtgatgaaaagcaggagcctccgcagccctaactcctgtctccaaggggtggctttgccctccccggggcagggatgagcactgagaccccattgctgtttcccagagatgcagtcgtcgtccacacatgaacatctgctgctggctgggcacggtggctcacgcttgtaatcccagccctttgggaagctgaggcaggcggatcacctgaggtcaggagttcgagaccagcctggtcaacatgatgaaaccctgtctctactaaaaatccaaaaattagctgggcatggtggcaggcacctgtaattccagctacttgggaggctgaggcaggagaatcgcttgaacgggggaggcagaggtcacagtgagccgagatggtgtcattgcactccagcctgggtgacagagtgagactctgtctcagaaaaaaaaaaaaaccaaaaatctgctgctcagtttgcctgtttttgttttttgtttttttcttaatagaacttgtcagtgtttccaaatcagcacacatagatctacagcggtcttttttgtttccctccagtggctttgttcattcacgtttggagtaagtgcttgcaggccctgctaagtgctgatcacggggacattgtcagtagatacattgctacaactgtgagcattttgtttataaggagccagttcacaaaaattagccgtcgggtgcctgtagtcccagctactcaggaggctgaggctggagaattgcttgaacccaggaggcagaggttgcagtgagccgagattgtgccattgcactccagcctgggagaccagagcgaaactccgtctcaaaaaaaaaggagcgggttaatttgccacgcagtgttttcttgtcattatttcataagcatagatggaaatttaggtggccttcactttttctatttcatgcagttttacatgtgcacttaaaaacacttgtgttggccaggcacggtggctcacgcctgtaatcccagcacttcgggaggctgaggcaggtagatcacctgaggtcacgagttcaagaccagcctaaccaacatggcaagaccccatctctactaaaaatagaaaaattagctgggcctggtcgtgggtgcctgtaatcccagctatttgggaagctgaggcaggagaatcactagaacctgggaggtggaggttgcagtgagccaagatggcatcgttgcattccagcttgggcgacagagtgagaatccatctcaaaaaaaaaaaaaaaaagaaaacccaaaaaacaaaaaaacaaaaaacacttgtgtgaaaggtggggaacagaggctcacgcctgtaatcccagcagtttgggaggccaaagtgggaggactgagccagggaggtcaaggctgcagtgagccctgatcatggtgctgcatgcactctagcctgggcaacagagcaagaccctatctcaaaaacaaggacaaaaaagtaaaactgcttgtgtgaatgtgtctgtaggataaacaccagcaggggaaattgctggatccaaggtctggacagacaggacttccgtggagggctttatgtgatgtaagaagagtccaattctgaattccaggttttcttagggaacatttggacaccttctttgaccctcccctgccggctgagaaggcagaagcaatcaggaagataggctttgggaccaacaacaaaatcttcctggagtttgaggagcccttctgggagccagactgccagctgatccagctggtgtgggaggacacgtcgcccctggaggatgctgcccctgagctacaggacgcctggttccggaagctcattggctttgtggtcctgcctgcctttgcgtacgtttgctccctgagaagttctgcgagtggctggctcataggccttcatctgatggaggacgcagcagtgtgtctgttcactgcagggtatttctagggggtttaatgggtagggttcccatgagcgcccccccaccaggtgctggctgcacctgggcctgacccctgcggggacagatggcgccctgctgtatgcgggttgtcagcgtccctcagggcccagaatgtgtttgtagacttgcaggcaggctcagtcttcagtcagtgggaacgtgtttactggaatgttgcagggagctggggcttgtagatacagaagaactttgaaacaccatctgcccatacgtggggagacaatacttaaatgggatgaaataattgaaggaaacattccgagaagtaatggaatgggagacccacagaggaagggctcccttgtatgggctggccctcttagaaacagcatatctagaccacgtggcgattcaggagttaatcccatgagtgatgtggggatggagggactgacttttccagcaagattaaaacacaaagacagcctgatatagtcagatggcagaaataattccacgtttccactgagagatgccgcaggagggggcaccaagttcttgttctgaaagacccgggatggggtgggatgtagggcatgcctgccagatggttcatagttgccatccactgggagcaggaggctgcaggctgcagaccagccctgcacctgtgacgggggaagcctgtgtgtctccttcacactgaagagaaaactggcatttcggccaggcgcggtggctcatgtctgtaatccctgcactttgggaggctgaggcgggtggatcacctgaggtcaggagttcgagaccagcctggccaacatgatgaaaccccgtctctactaaaaatacaaaaaattagccggtgtggtggcacgcacctgtaatcacagctactcaggaggctgaggcaggacaatcgcttgaacctgggaggcagaggttgcagtgagctgagattgtgccactgcactccagccttggcaacaagagcaaaactctgtctaaaaaaaaaaagaaagaaaactggcattccaggtgttgagataggcctgggttgctggtgggcagccgggtgggtgactgtgtgtagagccacctgctccaggcccagccccacctcctgtgctctcttgcttaggacatcagtgggaccctgagtgctttggtgggaccctgtctgtggcacggggtggtagctgtcccatccttttatatacctgcaaagggttggggatctcaaagagcttttgtttatgtgagtcacatctgtaaatttaccccatgataaataaaatatttttttgtttttgagatggagtctcgctctgtcccccagactggagtgcagtggtgcaatcttggctcactgcaagccccacctcctgggttcacgccattctcctgcctcagcctcctgagtagctggaactacaggcacccaccaccacgcccggctaattgtttgtatttttagtagagacagggtttcaccgtgttagccaggatggtctcgatctcctgacctcgtgatccgcccgcctcggcctcccaaagtgctgggattacaggcatgagccaccgcacccagtcaccacatgataaattaaattattatttgagatgatagtaacaaacctattctatgttagcataaataacatttttaaatgaatattttcttaaaatttaatgaagggtggcagcagttttgtcttttctgcaaaactttttttttttttttttgagacaaggtctctctgtcacccaggctggaatgcatggaatgcagtggcgtgatctcagctcactgcaacctctgcctcttgggttcaagcgattttcctgcctcagcctcccgagtagctgggattataggtgtgtgccaccatgcccagctaatttttgtagttttagtagagacagggttttgccatgttggccaggctggtctcgaactcctggcctcaagtgatccgcccaccttggcctcccaaagtgctgggattacaggtgtgagccaccacgcccggccttttctgcaaaactcttcagtgttacacttaatagaagacatctgggttctcaggcctcctctgcattcattctgtggtgatgttttattttggttgaaacataggaagaaaatgtggatctacatggatatgtggttggaagaaggagtgatatttgtttatttatgagatggagttttgcttttgtcgcccaggctggagtgtaatagctcgatctcggttcactgcaacctccgcctcccgggttcaagcaattctcctgcctcagcctcctgagtagctgggattacaggtgcccaccaccacacccggctaatttagtatttttagcagagatggggtttctccatgttggtcaggctggtctcgaactcctgacctcaagtgatctgcccaccttggcctcccaaagtattgggattacaggcgtgagctactgcgcccggccaaaggagtgatatttaaatagcttttgaaatgattgtgggtattctttgatactacctcaaaagtcaagatgtagtagtaattccttaaaggttggccaaatatgaaattggagagcacaccagtgaattttttgtcttctgtgacactgaaatctatttgtctgtcttgcactttaaatggatcttttatctatccatggctttagtcattggaaaatactaggctgggcttggtggctcatgccggtaattccagcactttgggaggccaaggtgtgcagatggcttgaactcaggagttcgagaccagcctgggcaacatagcaagacccagtctctaccaaaaatacaaaaagctagccagatgtggtggtgcgtgcctgtggtcccagctactgggaggctgaggtgggaggattgcttgagcccaggaggcggaggttgcagtgagctgtgatcaagccactgaactccagcctgggcaacagagggaggccatgtcttaaaaaaataaaatatatatatattggttcatgtagtcttccaaatgttgatacatttcattatacaatataaaaaatcaaatttattaacataccactgatctcagaaaagtctttcaggattgggaagctgttatgctgttggtggcagaaacaagcttttctaaaattgcagtttttgcttgagagcttaaactttgtcattgtctataaatactatttgttagttgttttcctgtgatagagacactttgctcattttcaagaaaatgtcaccaaataccacaccatgatttgtctgactggtgctctcctgagttctgagcctgtctgtgaaagacgcaccagccccgcctggaatagcgtgtgctaggtgagtcctccacggccaccgtgcaccacgcgcagcaggagtgttctatgcggatcccgcttctccacgcagattattattattttttgagatggagtcttactctgtcgcccaggctggagagcagtggcgcgatcttggctcaccgcaacctccacctcccgggttcatgccattctcctgcctcaacctgccgagtagctgggactacaggtgcccgccaccacgcctggctaatttttgtatttttattagacagggctttaccatattgaccaggctggtctcgaactcctgacctcgtgatccgcccgccttggcctcccaaagtgctgggattacaggcctgagccactgtgcctggccctccacgcagattattaaaaagacatgaactcgaggatcaagatttaataaaattagtaacttttactgcctttgatcaaggacattctcaagtgaaagcagcatttttatttattttttaatatatatattttaatttttatattcagaggcagggatatacgtgcaggtttgttatgtagctaaccgtgtatcatgggggcttgttgtacagattagttctatcacccaggaattaaacctagtacccattagttatttttcctgctcctctccctcctcccatcctccactctcccataggccccagtgtgtgttctcatcatttagctcccacttgaaagggaaaacatgtgctgtctgcttttctgttcctgtgttagtttgctaaggataatggcctccagctccatccatgtccctgcaaaggacatgatctcgttctttgacggctgcaaaaagcagcattttgtaaccctcatgtgcagtggtgggaacgcctgtaggcgcatgtggatcctgttgctgcctgattctgctaagggacggtttacctgccctggctcttgctcaggtgcaaatgtcggcgtggtggaaacagcaaatgctgtcttagcgtctgaagatggctgtgaccttgagggtccctggcagggccttcggacccccaggactttgccgcccacactctgcgaactgctgcctgagggacgctctgtggggtggctctttgtgcctctggggcccctgctccatcagtggttccttgctgagaaacgcgggcggagagctgggctggacactgtcatcccggggctctctttctccatgcaggtctggtcatcccagggctctctttctccatgcaggtctggtcatcccagggctctctttctccatgcaggtctggtcatcccagggctctctttctccatgcaggtctgtccacgttctctgtgggttcattgccggacttgagtctgagttcatggagactctgtcggatgaagaagtacttctgtgtctcacccaagtgctccggagagtgacaggtaggtactcaccacacacgctggttcctgcctctgctcgttactggttgatctctaaacagaaactagactttgcagaacagagaaaaactaaatttggctgactctgtacatctccagggaccatccttcctcatgttagtggggtcagtaacagttcttacctccgaggaactgaaatgactttgcaaacacttcctacttctgccaccaaaataaatgccgttgtccttgatcagatttttacagaggaggaacccaaggctcggaaacagtagctgctgctgtcacagctcccgggtcccggcccgctgctgtcgggtcagaggcccccgggtcccagcccactgctctcgggtcagaggcggcaggaagagcctctcctgacacactctgctgctcacttttcttcctgcatagcatctttcacgttaaactgctcaatctttaaaaatgacaaaaacagttgcagacctgtgggtgagagttctcggttaacatgcagtgtctctgtggctcaggaaacccacggctccccgcgcccaagagcgtcctgcggtctcgctggcacagcgccccgtacactagggggtcctacagctacgtggccgtgggcagtactgggggcgacctggacctgctggctcagcccctccctgcagacggcgccggcgcccaggtatgtggcgtgccccagtcggggggcgtgggtcccgctgcagaggcccccgccgagtctgggcgctgcaggagcaccagccagtggcgggtgggctgggatcccagactcggaagccattttctttttgaaactaagaataatttttaaatgtactgtttttgtgagaattatacacacacagcttaaaaagtcagagaattctgcagtgctaacaaaagaaaataaaaccaaaatggcaggtggagccccttttgcccttctcagacggtgctcccagctgattctcatcgctaagtttactgcatctctaaaaacatgtttctgctaccacgttttgagatacatttcctgtgcaaaatgtcaaacatactaaagtagggagaacctttcagtgcccaccaggcttcccctaagccaagcttctccccgcccacccccgcccttttttattaagcatgactcacacaggacattgtctcacctcaggatattttggtgcctgtttctaaaagataaagacatccttgtaaaatacttaactatgatgctgagggcagtggctcacacctgtaatcctagtgctttgggaggcctaggcaggaggtttgctgaggccaagagttcgagaccagcctgggcaacatagtgagagtcggtctccacacacacacacacacacacacacacacacacacaagtagccaggcatggtagcatgcacctgtagtcccagctgctcaggcttgggcccaggaagttgaggctggagtaagctatgactgcaccactggactccagcctgggtgtcagagcaagactctgtctcagaaaacataaaaataaaaccatataactatgataccatttacccacttaaaaattaataatcattcctcaatatgatccaatatttagtcactgttcagttttccaatcagctcatggtctctcttcagtgaacctgcttggaaatgttgcttcttgagttttcagtttcaggtgttactaattgacttctcattacatcagataaggctgtagctttcttcctctgcccacccgactcccagatccctcccccaccctccccatatgtgatccaggagttgaggccagatcactattcagtgtttacaccccttgttttctctaaaagctgttcacacctgagctgcggggtgcagtggcttcctttcccacccattggtggatgcgtgtgagccgtcttcccacccgttggtggatgcgtgtgagccgttttcccgcccgttggtggatgcgtgtgagccgttttcccgcccgttggtggatgcgtgtgagccgttttcctggctgttgatggatacatgtgagctgttttcctgcccgttggtggatgtgtgtgagctgttttcctggctattggtgggtacacgtgagcccttttctcgtccgttggtggaggtgtgtgagctgttttcctgcttcttggtggatgcttgtgagccattttctgtgtttccttgcccagaccctgctgtctgaattgcatccccattctaaccctggctcttctttgcagctccagatcctgtttgcgggggaagccacacatcgcacgttttactccacgacgcacggggctctgctgtcgggatggagggaggccgaccgcctcctcagtctgtgggccccgcaggtgcagcagcccaggcccaggctctagctgggcccagcctactctgttccacccgtgtcgggggtaggctgggaccctcatttcttctgacagatttcagtctggcttgaaatttggggatgttaatgagggtcctctggtttttggtaaccagggccaccttctcagttcttgtgtctgttattggagtctggccagggttgacttgagctgagacaccagatgctcacggagatgctggacacataaagcaagttacagccacagctcccagagccatctttcctgccttgaggctgtcctgtccttcctgccttggggctccagcatctgctgctgttgtctttgtcttgctccgggcccaggttgcacagggccctgtgtctccaatccccacaaccacacatggccccacactctgggttccctgaactctgggtcctgtagttgcttctcgtgcccctgcattctcgtggctgctgcctgggatgacagcatagctgacaaccctcctcttgctcatcaactgatgctgcatcaggcgcccacttcacaggttcgcagggcactggggagatgaggggtgccctgccctgccctgaggccgggggttttctggaggaagtgaatacataataaggcctgaaaagagaaacggtgcccagcccatgggagccagggagtgtgtggggcgtgggcagtgctggggtgagtgtgctgggtgctggggggtgttggggtgggggcagtgctggggtgctggggggtgttggggtgtgggtagtgttgggtgctggggggtgttggggtgagtgtgctgggtgccgggggatgttggggtgtgggcagtgctggggtaccacgtggtattggggtgaatgtgctgggagtccaggcccctctccggcccctccaccccagcctggatctgcagggctcctctcttctcaccctctggtctctgttcaaattgcccctcctaggatgttccctgaccccaccctcccgagccccagcctccctctgtagccagcagccccacctgcatttgctgtgtcaccagactgttctctctggggacacctgcccatcagacgcctgggagagaaattcagattccacagggttttattgtggttttgtgctcctaggaccatgagggaatgcagcttccttaaagtacaaaaatctttatcattgacatttagggtttcatcaattctgtatcccagggagaaaggcagcaagacccaggctgccccctctgggtgtgagggaccggcttggtgaggagggggcttgggggcattgacctagcaggaggccctgactgcccccactcagcccagaaaccacctgggaggcttctgtcttccacggaggggccactctctcatgcccaaaaatatttacagctaggcgttagggtattaggtggtttaagaacatcagaaccatggcaatatttttgtacttagaatttgagcatgtttggtattttcagtagct

SEQ ID NO: 15 - DNA, 16 bp, Homo sapiens
cttaggttttcttagg

SEQ ID NO: 16 - DNA, 10 bp, Homo sapiens
ttttcttagg

SEQ ID NO: 17 - DNA, 27 bp, Artificial Sequence - Synthetic
  Synthetic
aggcagcctttcccggggagacctttc

SEQ ID NO: 18 - DNA, 23 bp, Artificial Sequence - Synthetic
  Synthetic
tctccatgaactcagactcaagt

SEQ ID NO: 19 - DNA, 21 bp, Artificial Sequence - Synthetic
  Synthetic
tccaccaccctgttgctgtag

SEQ ID NO: 20 - DNA, 22 bp, Artificial Sequence - Synthetic
  Synthetic
gaccacagtccatgccatcact

SEQ ID NO: 21 - DNA, 16132 bp, Mus musculus
tgtacttatgattagaatggcttacaggctttggcccagttaacccaatagtgtctgtctcctgatagcaagttcaagaatccaatagttcagtccacaaggctggagggctcagctggtcttcagcgtagttggaatccagaagatgtagctctaatattgatgaggaaatcctcaggatcaggctgtaaaaactggccagtgagaacgagggtgagcaggcagaaagcaaagacttctttcttctgtgctctttatatagggtgcatgagagatttttcttggttggatcattcgaggtaagaagaccagatataggcttaccttcctacctcaaatgatccaagaaaaatccctcacagatgttccctgtgatgtagtcacattggcaattaagaacagccaccacaactatcaaggactttggcaaaattaaaaaacttctgtttcagaggagaccatcaaggtcgagacacaagacacaagcaagatgagagaaacttttctacaattcttacatctggtaaaggtcttgtgtcaagaacacataacacaacactcttacaatggaataggaaaagatatcggaaccacaaagagcaaaggatgagcatacatgtcttcaataacgaagtaattgtttgagggatgttagaagatgcccaacatcattagcctttagggaaatacacatcaaatctacttcaaacctattaggttgactacaataaaaaaattttttaagtcaacagtgagtgtgggaaagcatgtagagaagttggagcctggctggcaagatggctcagcagtaagagcactgactgctcttcagaaggtcctgagttcaaatcccagcaaccacatggtggctcacaactaccggtaatgagatctgacaccctcttctggtgtgtctgaagtcaactacagtgtacttacttataataataaataaatccttgggctagagcaagcagggactgagcaagcagagttgatgggagtgagcggggttgactgcaacgagcagaggtcctaaaaattcaattcccaacaatcacacgaaggatcacaaccatctgtacagctacagtgtacataaaattaaaaataagtaaattttttttttttaaagaaaagttggagaggcaggcagatttctgagtttgaggccaacctggtctacaaagtgagttccaggacagccagggctatacagagaaaccctgtctcgaaaaaacaaaacaaaacaaaaaagaaaagaaaagaaaagaaaagaaaagttgtttggggaatatttttagacatcagtgagagtagaagagagggatgtcacctccagagagctggagtcaggacagctcctgaaaggctttgatgctgagtcttgtttgtattttaattatatattccaaagccccaaggtggggtgagccagcaagattttacacagaagcctgtgtggccatcagcaagttgttaagggcaatcaatgactcattgtttcagatagcaattgaagtcatgcttgccaaatatgcagaacaagcagtgctttaagtcactaaataagtcaataagtccgtacctaacaattgctctttcctgccttactcagtgatgggagtacaaattggcgtggccacttggcctttgtgaggtgcatgtagatgttcctggagactgaactctggccttacactaccttgtcatggcctgcccttgtggcgaaagttaaaacagcaaaccaacgcccaggtgcatactcaagagaaatgaaaacatggctccacagaagctcctaggtacacagcagcaataatcacaggactaaaaaaaaaaaaatagaaatagcctaaatgtccatcaaacattctatacttgtataacagacactggcttagttagggttttactgctgtgaacagacaccatgaccaaggcaactcttataaggacaacatttaattggagctggcttacaggttcagaggttcagtccatcatcatcaaagtgggagcatggtagtgtccaggcaggcatggtgcaggaggagctgagagttctacatctccacctgaaggctgttagcaaaatactgacttccaggctgctagggtgagggttgcccatgcccacagtgacacacctactccaacaaggtcacacctcctaatagtgccactctctgggccaagcatataaaaaccattgcagatgcctaagttttgtttttgttttttaaagatttatttattttatgtgagtatactgttgctctcttcagacacaccagaagagggcatcaaatccctttacagatggttgtgagccaccatgtaggtgttgggaattgaactcaggacctctggaagagcagttagtgattttaactgctgagccatctctccagtcctctgccttagatttttttttttttttaagatggggttttccatataactcatgctggcctagaatctgcccaagctttgcctcacaaatgctgttattaaaggcaagcagaaccacacctggctacttaggtattaacatattcaaatagataccactgactagaatgtgcggtggagactgaaggtgtagctcagtggtaacattcttgcctagcatgcagaaagccttgaagtccacagtaaatacggcatatacatgatgaaaaggcgaagtactaacacacactgtatgtggaaaaccttgagagtgtgcctcatcctctctaactttttttttttttaaagatttatttatttatttattatatgtaaagtacactgtagctgtcttcagacgcaccagaagagggcatcggatctcattacgggtggttgtgagccaccatgtgattgctgggatttgaacttccgaccttccagcccccctctctaactctttatatgaaccagccagaatagggtgatgagagggttttggactcagttgtgatgatggctgtacaactctgccaatatgctgaaagccgctggcttgctctcgttatgtgggtgaagtgcatgaaaattccatattgatatggctatttttatttatttattttttttggtttttcaagacagggtttctctgtatagccctggctgtcctggaactcactttgtagaccaggctggcctcgaactcagaaatccgcctggcctctgtctcccgagtgctgggattaaaggcgtgcgccaccacgcccagctggctatttttttttttaaacgaacaatcccagtttggtttagtcacctggaagatattttctgtggtgtcggacagccctcctgcctttggacaagcatgccatggtatttgcatactctccagtggtctgtttaatgagcatatgatgaccccagctgcagggcagcctttgcttcagaccggtgcactatgtgtaggggacaagaagcactattgtctgcttggggccagccctggcccattgacagggtggccaagaggagataaggggccggtgagggaagtgagtcaggtgtagtggtcaggggaaccttgcagctctgactcactaggaatccgattgctgctttatttatacacgtctcacagaacaaagacagactaatgatcatccaggagcacagattagattagttttctctgatttttttttttttttcattacacgttcagggttacaagttcagtcacaattagaaagtactcatagaactgattttatttttattattagccctaaacttcagttcaaataatctacggaatgtctccactaaagcaagcacatttattatttgcagcctgcttttaggatggcagcacttgcaggtttgtttgtttgcttggttgtattttttttttttagatttatttatttatcatgtatactgtgttctgcttgcatgtatgcctgcaggccggaagggcaccagatctctcattacagatggttgtgagccaccatatggttgctgggaatagaactcaggacctctgaaagagcagccagtgctcctaacctctgcactatctctctagcctaatatctgcagttttaaaagcatctcagacacacagaaaacatctgagccagtctttttatgaacagcaaatacaaacagctaactcctattgtgaaccatttcaccatctcacttgaaagtaggaaaaggttattttagtcaatctatcttatttattgacgtctattcctcctgggtatgctctgtacagccccctttctttaaactacattttcagagagctctaagcatatcataaaaagaagcaaggatctgttactaattctcttggtcagaattgtcagctatctctgtttacccctgtgccaatcatactgtttgaatttgcttagctgcggataccccaagaagattcctggaatcatggcctcattataaatgcctagcttttgcttatctgagtgtcttagtcagggtttctattcctgcacaaaacattgtgaccaagaagcaagttggggaggaaagggtttattcagcttacacttccacagtgctgttcatcaccaaaggaagtcaggactggaactcacacagggtgggagcttggaggcaggagtgcaagcagaaatcatggagggtgctgcttactggcttgcttctcttggcttgttcagcttgcttccttatagaacccaggactaccagcccagggacggcaccacccataatgggctgggctctccacccttgatctctgattgagaaaaagtcttacagctggatcccatggaggcgtttcctcaagggagattcttttctctgtgataactccagctgtgtcgagttgactcacaaacccagccagtacactgagcttatcagaatcttcaaggcacaaggaagacttccaaatagtggctaaagctagttccataaaaccaggaaaagacataaaacgagtcacaatgcagaaagtctccaagaacacaacacacagaaaccaaaacctaaaactgagagacagacagaacgacagcaagctcagcattccactcagcttggctggaactgtgtcaagcagcttgctagctttatgacttttgctatttccaggggatgtggctgtttaaactgtccctgccctgtaccgctagaggcccagcgtgttactaccagccactttgttaaaagtttcaacttcgactttcctgtatcccacagaaattaaaataggaattagtaaatgaactgtctactcacagtttgaccgcagactccgcctttcctctagcagggtcacaatggtacaggaaaggccaggcatggtgggtgcatggcaaccccatgagggcagtgcagaaaatgcatagcaagtttcaggccagcctgggctaactgagatctccatttttcatgattgtgatccctcccaaacataaaaccaattatgtagaaaagattaaagagaagctaggaaatatacacattaacttatccagtctgtaggggaagggtacttgggtcccaggctgttctctattaacagaattttaaaggatcttttatttaccttgtaacattttcatacatataaatgagatctcttgatgatatttatttttaactctctttcccactcccccaagcatcttcaacatatcctctccctcttcgtggctcctcctcctttttgtaattcactgaatctaattagtgctgcttgttaggatgttgatagatcttggcttgatactgtgtaggtcatcattgctacagaatgttcatggccacctcatggccagaagtcagcatctcataacattcgtcttgatcttctggttgttacatttttaagaaaaactttaaaaatatttattagcttttagtttatgtgtgtacatcatgcatgcatggttgcccatagaggctggaagggggattcctggagccagagtgacagggcttgtgaaccacctgatgtgggtgctcagctgggaactgaactccagtcctctggaagagcaacgtgggatctctccactccatggatcttacactctttccctttcctctccttgatgttccctgagcgttgagggtgggggtggggaggctgagacagatacttcatttaatgttgtacactcataggtcacttattctcagctcgcggactagttgtgagtctctgtactagttgctgctcactgcagaagaagcacctctggccaaggttgagagaagcatgaaaacccaacattttaaaggcaaaacccaacatttaaaaggcagtctgataacaggatcatttagcaaaacagcagaaggtccctgacagtcctgtagcatgtgggaattgacacagagcaatgccgctgataacttttttcctacagcagcctgcatagcagcttttgacactatgcaaactagcaagcaggagggaagttttcatgtctgctccaacttgattttctatgtcttgtgaaaccaacttgttagaagttttaaaactgtgtagaggaaaatagtgagtattaaagactggcatttttaatgaaccagaccaggtagctctttgtctccaggttagtttcctcaatgtagggtgggatgcttttggtatctttgcttgggagggcagcttcaactcccaactcccaagatccagttaaacactctaacttagaaagtgaacaagagtgatgttttgtagctgaggggagggcatgtgcctcctgcccttaccatatatcctcttcagacgcggatttatgcccagttcccatctatgtcagagccatcaccccctccccatagcatacaaccagtgagttttatttctctataccacagctagtagctctgagtgaatgaggggatcaagtacgttggtcaaggacatttttgcccctcagcctgctgagtggttggccaaaacagtgttcttagcttacaagatccctgtctttgtgctttgggtcagcatggttacaggacagaacttatcttctaagcgggttgattcactgtgagccgacaggccagtggaaaggagactagagctatggcgcactctatgctctgatacgaaccaggaaggatgcttctggaagactatgcatctctatggggttctaggctcacactgcctagtagtagctacagccacacccttcttaagtagactataggcgggtccaaacaggaggagggctgctcagagcacttggagcttgagcgcttctgggagccgcaagcctacgtggaccacacagtgaaggccaagggacatccatctcctctccttcagtccagttccgaatgttcaccaaggctttcctcccgtcttggcccctcctctgctccccataggccccgcccctcgtcccacctccagtcccacgcacactcactcgctcctttctacacaaccccgccttcttccccgcctctggccccgccccgcccgcctctgctaaggcggcctaaggccaggtctctggaggaccctgagctagccatggcgttccctggcccgcgggtgctggtggtgggcagcggcatcgcggggctgggcgctgcgcagaagctctgcagccaccgggctgctccacacctgcgggttctggaggccaccgccagcgccgggggccgcatccgctcggaacgctgcttcggtaaccgctccttggtcgaccccagaccccatccggttcctactgagccaggaagccttccttttttggctcccggagtaaaggaacacaccctccgttcactgagcctggaaagtcaactgcctaaagttaggagagtactctctagggaaggcaacctagggtcccgcagttgctttgctcagctttggggtttctcgggtttctccttaagtgaaggaagcctgtacaggctgtctcgcccctccccaggagttcccgaaatctggtctgaccaagccatgcctttaggaactactttaccctgaggaacaggacccttggtcctggtatacaaggggaccatgtgactctcaaggtgttcctagagtatgcttgcgaaagggtggggctgctttttctggggagagggcctccaggtagcatttcctgtgccctcctaggtggtgtagtggagctgggtgcacactggatccatgggccctcccaggacaatcctgtcttccagctggctgcagaattcgggctcctgggggagaaagaattatcggaagagaaccagcttgtggatacaggagggcacgtggccctgccctccatgatctggtccagctctggaacaagtgtgagcctggagctgatgacagagatggcccgtctgttctatggactgatagagcggacccgagaattcctgaatgaatctgaaaccccaatggccagtgttggagagttcctgaagaaggagataagtcagcaggtggctagctggaccgaggatgatgaggacaccagaaagcgcaagctggctatcctgaatactttcttcaacatagagtgttgtgtgagcggcacccacagcatggacctggtggctcttgcaccgttcggggagtatacagtgctgcctgggctggattgcatcttggctgggtatgggccatagccctgccagctctgtccctactgtccaggcagaggcactagactggagaaaggagggagaaggggagagaggaggaggcttggagcgggcaggtgagggcactgccctccccattcagagggattggtttctctttctcctagttgtgtttaagcaaagattcgtctagggaattgaggaggaagtatcttgaatgctcatttcttctgatatcctagggtagctcctgtgccaggtgttactcaagggccttcaagtgcagagaagccaagcaggctccttccctatggcaggcaggcttgtgcttgcttctaggaacaaagacattgattgggcggggctggtctcctatacctcccacccacacccctaacatcaggcccaaggcttctgtctaggagataggatttgtgatgtaggaagtgtgcaaacctggggctggtgtggtggtggggaactccggttctcatgggaaccagtcagtgttgtgttcattgacttatgggaccctcattagtccggatgcagatgttaatggttctgtgttttaaggggttgcctctctttctggctctgagcgatgctcctctggatgctggtggcatcttgggtggcttatttctgccagttccccttccagggcctgtttcacagacacatcaacttctgtctctcctagtggctaccaaggacttaccgaccgcatactggcttccttgcccaaggatacggtcgcttttgacaagccggtgaagaccattcactggaatgggtccttccaggaagctgcttttccaggggagaccttccccgtattggtggagtgtgaagatggtgcccgcctgcctgcacatcatgtcattgtcacagtgccattaggtagaaatcacttatcctgattaagctgcctgtctatatccctatcccaggcatcacaggcctctggttcttctccttctttggctctggatatttattgagctgctgagttcatgttttcatgctaaggatctccaggtgaataaaagtactccaccataatttttggcagttgtagccgtcctgcctcacctggagtcactgtgctgccttccctggctgatactgtcttgtatagagaagccattattaataaatgctggtgggaactcggtagttcttacttccctgtttcagatggtgttacatgaaaatctcaaagtgcttatgtggcttgttcttgaagatctagatagaatgacttttcttcccaggacctgtgtattcgggaactgtctctgtcatgtgcctgggtatggccctgcaggcttcctgttgtactgattttcttccttccttttttaaactttaatttgtcttttgagacagggtctcactatgtaggcctgaatggcttggaactcactgtgtaaaccaagcttgtgtcaaactcagagatctacctgtttatgcctcctaagtgctgggattcaaggccttcttgctttcgtactggtttcaagcaaatgttctctcctctgggttgtctttgttgtttgtctgactgtgggttgtttttcttttttcttttttttccctacttagacttatctaagtgtattcagtggcagagtgtttgcatggatgaggccctgggttcaatttctagtaaagagcaagacaagcttcctcgtgaaatctgtgtggtggctaccacagactgcccctgagtagtgtgactggcagttagagatagaaagtcaggagtgagatgttcctcagagtcccacttaactgtccctctgtgaccagggagtctgtagtttccctccaatatggtagcagtcaaccttgagttctgattggcaggtccagcctttggaatttgtctccaggtacccgaatgaatgtacgggatgtttagaagcacttttctctagacaatcaggacagattcctgttgatgtagattctagttaaaaccgaacttatttatttacttatttcttcctcttcctcttcctcctcctcttcctcctcttcctcttcttcttccttttatcatgtagcccctgctgtcctggactcattatgtagaccaggctggctttgaactcagagaaccatgcctgtcactgcctcctgtgtgttgagattaaagacatgcatcacaagacctgtcttaaactacgtcctgttctgaattgcaggttttcttaaagaacaccaagataccttttttgagccaccactgcctgccaaaaaagcagaagccatcaaaaaactaggctttggtaccaacaacaaaatcttcttggagtttgaagagcccttctgggagcccgactgccagttcatccaggtggtgtgggaagatacatcgcccttacaggacacagccctctcactccaagacacctggttcaagaagctcattggatttttggtccagccttcttttgagtatgtatgcatccaggagttctagggaatgcacctgctggctcactagtttagtctgttggagtactcagctgtgtatagttcattgtcaggtatttctgagtgtttgattggaggaggagcatgtgctcctgaagtgttggtttcagatatcctggctccctgagagacaaatggagacccagaatgtacttttggactccagggatatgtgtgtgtgtgtgtgtgtgtgtgtgtgtgtggtattaaaacttggtatttgaagtatggatctgtctatacctgtagaaatagtacttagatgggagctaaagcgcctcctacataatggaagtagagaccctcagttggccaggcatagtgatccactcctaatagtaggaaagctgaggctggaagaacatgtgtttgaatatgtcagactctatctaaaaataacagacagacctacagatgaaggtctcaactagtggctcagccatcctataaaggagtgtactttttctaggtactaattctaggtattatgtgtcccccaggcatagcctaggacaaggactgatgtctgataatatcagaacacactgagctgttgtcatgtgaaggaagggagttcacattccacttagagatctggcaagaaagggccctgttcttagcctaaaaagactaggatgggtggcagcaggagcccacagctgcagagcagccttccacccggaaggaaaggagagagggagttatttccacaggagctcaactgactttcaagatgcaaatagaccgtgctgcaattgtctgtttggaacttagactgtgggtcccagtagcgttgaaaaactacattttttttgaaaaattatatcaattttattctcttttctgtaactacctctcctcttcctcctcctttttcttcttcacagggtgtatcatgtggctgctttctctgcgtgtgtgtctgtgagcctcatgcctgcagagcttcagaagccgagagcgggtgccggatcctttgtagctccaggtcgggaggtctgttagctaccacgtgggtgctggaaactgaaccctggtcctctggaagggaagccagtatttctttaccaccgagctttctcttctgctccgttattttcttttcctaatcctgtatctgtgtggataaagatcaatataaatgagcgcagaagtagctaagactcccgtgtattctccttagccagttatcaatgagagtcatgggcaagcaaaaccctgccaccttttaaattcagtttgagaaaatatgcttgtcccttcccgtctttgtgtgcgtgtgtgtgtatgtgtgtgtgcttactaacgtgtgggtgtggggaggtcagaggagggtctcggggatcttcctctatcgctctgcaccctatgttttgaggcagggtttctcactgaacctgtagctcattcactgactttcacctctctgtaattggggtcattcagaaatgttggttcatgcagtaatgccaagagttaaattcatcaatatcacctccgatgtcagagaactctgtcagtatcatgaagctgttacactgatggcaacagatacaattttgctgaaattctgctttttgctcgagagcttacatttcatttttattaatacatactatgcactttttttttcctgagaaaatatctgctcagtaccatggttgcctaggtgtccttgtaggtaaggaaggtgtgtgtgaaggaagcagctccagactcgaagacagcacaccagaaacctttatacctcttcccgtttcttcacagacatgggaaaaaatgcactcgatggccaaaatttaataaaatcaataacttttttttaaaaaaagattttatgtatgtgagtatactgtaactatcttcagacacaccagaagagggcatcagatcctattacagatggttgtgagccaccacatgggtgctgggaattgaactcaggacctctggaagagcagtcaatgctcttaaccactgagccatctctccagctccccaaatcagtaactttactatttcattaaggatcttatcaagtgaaatcagcatccccgatgccgcccccccacccccctgtgtgcgtggtggtgaagaacaggatgcctactggtctggcttgttggtacctccatcctctgagggacagtttcctgctctgctcttgcacatttggtactagtgacaaggtggaaacggcacaggctgctttagggttgtgaaaatagccagatacttcgagagctaagactctaggagagctctgggggcctgtggcggtcctggcatagtgtttgtgtggccagcagaccgatttcccatcttatgcttatataggtcttcacatgtgctctgtgggtttattgctgggctggagtcggagtttatggagactctgtcagatgaagaagtgcttctgtctctaacccaagtactccggagagtgacaggtactccacctccactatatatatgtatgtatgtatatatatacacacacatatatatatacatatatatattctgtattcctcattttttattttattttttatatcctatttgtttattttatgtatacaagtatttatctgcatgtacacctccatgccagaaaagagcatctatcatattatagatgtttgtgagccaccatatggtcactgggaattgaacttaggacctctggaagagcagtctgtgatcttaaccactgagccatctctccagcccccagctatatatcttattggccaatctctaaaaaggaagtaaacttgtagcagagaaggaattcctctctgcacatctccaagacaactttcttcctgggcactgggcaagccatggtttgcaaatacttcgtaattttattttctaaaatcttctgccccaaataaatgcagttgttgtccctagtcagatgctttcaggtaaggaaacttacaggaggcatctcacggtgttgcttattggcaccagctgtccctgtgtagtgctggagagcagctaagacaggatgcctcttaggaaaacaagcattggcttgttaccacatctctgtgtttcaggaaacccacaattgccagcagccaagagcgtacgaaggtctcagtggcacagtgccccgtacacccgaggttcctacagctatgtggctgtgggcagcactggcgatgacctagatctgatggctcagcccctccctgaagatggaactggcacccaggtatagtggcctgccttggtgtggggcaccggggatcctgctgctaattaaggcctaaaggtgatgtggtgtgagtgatgggtggggctgggatctcacagacacagagccagtttcttttgaaaataggagtaacttcaagttgaatgtgctggcatacgctcacagtctcagcactcaggaggcagaggcaggatgactgtttaagagtccacagacagcctggtctattgaacaagttagtggggggggggggggacaaccaaggctaataagactatgtctcaacaaattcagacccaaactaagcacttaaaatgtgttattcctatcagaattacatggcttggcttaaggcattgtatggtgctgactacagaaataaaatgaaacaaaagtagccggcagggagttctcccagtgtccagactatgtttccagctgttcttttttttttttttttttttttttttttgcattggggctgccctgagtgagcacaccaggcaagcatgctaccacctgattctagtatcaaaagtctaggtgggcccaggggctcatgcatctgtaatcccagctaaaaagatcactcccgattttaggctagtctggtttaccaaatgagagccagtccagccagatccacatgctgagaccggttccaaaacgatttaattatggagaccatcagctggagagaaatgggtagagcagtgggtgctgtccccttcctctgcccactacctctcccctctaccacagtgggcagaacccactcactctcccttaacttactgagtagttatccctgtagatagttcagtgcgtatctctaagacagctactcagtattacccggtattcactggttcagttgaccaggtgtctcacggtccctgttagcttgctttttttcttttctttttttttttttaaattaaggtattttcctcgtttacattttcaatgctatcccaaaggtcccccatacccaacccccccccccttgccaaacgcatgaaattcaagaagaacgaagaccaaagtgtggacactttaccctttcttagaaatgggaacaaaacacccatggaaggagttacagagacaaaatttggagctgtgacgaaaggatggaccatctagtgattgccatatgcaggaaaccatcccataatcagttagcttgctttttgtcacaccacttttagctgtaggcagggacttcaaaaccaatactgtgatgtattaggtttagctgctgtctcccatgatagtttagcaactcatctgatcatggttcggggacctaccacccgggtgtttcttagcattcacagctgcctggcacgggcagcgatggcttctgcccatttgtagatgcccatggatgggcaggggctctgttgtctgtgtgtatctccacagctgtcccacttgcatccctgttctaactgggccttttcctgcagctccaggtactctttgctggggaagccacacatcggacattttattctaccacacatggggccctcctgtctggctggagagaagctgatcgcctcgttagtctgtgggactcacaggtggagcagtcgcggcccagcctgtgagtgcctgctgctctgctccaccaaaagggctgaccaagctctgtttggtttgaggtttggggatcttactggtagctttaattttggggaagtggttttgtttttcttatctgttggagtctggcttggtctgatcatggatgccccgttaagtgttgtacgcataaagcagttaacattg

SEQ ID NO: 22 - DNA, 45 bp, Homo sapiens
cgtcaccgtgcccttaggttttcttagggaacatttggacacctt

SEQ ID NO: 23 - DNA, 35 bp, Homo sapiens
cgtcaccgtgcccttagggaacatttggacacctt